(19) United States
(12) Patent Application Publication
(10) Pub. No.: US 2011/0313032 A1
(43) Pub. Date: Dec. 22, 2011

(54) COMPETITIVE INHIBITORS OF TYPE II DEHYDROQUINASE ENZYME

(75) Inventors: Cóncepcion González Bello, Santiago de Compostela (ES); Verónica Filipa Vieira Prazeres, Santiago de Compostela (ES); Sonia Paz Gómez, Santiago de Compostela (ES); Cristina Sánchez Sixto, Santiago de Compostela (ES); Lorena Tizón Valverde, Santiago de Compostela (ES)

(73) Assignee: UNIVERSIDADE DE SANTIAGO DE COMPOSTELA, Santiago de Compostela (ES)

(21) Appl. No.: 13/141,614

(22) PCT Filed: Dec. 23, 2009
(86) PCT No.: PCT/EP2009/067858

§ 371 Date: Sep. 12, 2011

(30) Foreign Application Priority Data
Dec. 23, 2008 (EP) EP08382085.2
Jun. 26, 2009 (EP) EP09382104.9

(51) Int. Cl. A61K 31/381; C07C 62/26; C07D 333/60; C07D 409/12; C07D 333/54; C07D 333/24; C07C 51/09; C07D 307/00; C07D 495/08; C07F 7/08; C07D 409/10; A61K 31/19; A61K 31/192; A61P 31/00; A61P 31/06; A61P 35/00; A61P 1/00; A61P 1/04; C07C 62/38

(52) U.S. Cl. 514/443; 562/508; 562/463; 549/58; 549/51; 562/462; 549/59; 549/79; 549/302; 549/44; 549/214; 549/4; 514/557; 514/570; 514/569; 514/444; 514/438

(57) ABSTRACT

The present invention is directed to a compound of formula (I), its diastereoisomers, its enantiomers or its pharmaceutically acceptable salts or solvates, formula (I), to procedures of obtaining the same, to intermediates thereof, and use as competitive inhibitors of the third enzyme of the shikimic acid pathway, the type II dehydroquinase.

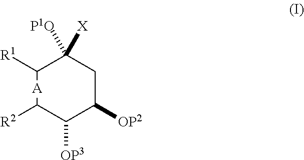

### FIELD OF THE INVENTION

The present invention relates to compounds of general formula I, to procedures of obtaining the same, to intermediates thereof; and use as competitive inhibitors of the third enzyme of the shikimic acid pathway, the type II dehydroquinase.

### STATE OF THE ART

Although nowadays enormous effective chemotherapeutic agents have been developed, the number of deaths among hospitalized patients infected with resistant bacterial strains has increased dramatically. This fact is especially remarkable for important diseases such as tuberculosis, where the current therapies become less efficient. Their effects are particularly strong in people with a compromised immune system such as HIV patients. The synergy between the AIDS epidemic and increasing surge of multidrug-resistant isolates to antibiotics leads to the alarming conclusion that antibiotics are loosing their effectiveness. It is therefore necessary to discover new, safe, selective and more efficient antibiotics to face this problem.

For example, selectivity can sometimes be achieved by using compounds that inhibit one of the biosynthetic pathways present in bacteria. Thus, there are antibiotics that interfere in the protein, lipids or catetenoids biosynthesis, etc. In bacteria, there is a metabolic route, known as the shikimic acid pathway (Haslam, E. The shikimate pathway. New York: Wiley; 1974), through which chorismic acid is biosynthesized. The later compound is the precursor in the synthesis of aromatic compounds such as the aromatic amino acids, folates, ubiquinones and certain vitamins (Abell, C. Enzymology and molecular biology of the shikimate pathway. In: Sankawa U, editor. Comprehensive Natural Products Chemistry. Oxford: Pergamon, Elsevier Science Ltd.; 1998. p 573). The shikimic acid pathway is present in bacteria, fungi, higher plants and has recently been discovered in apicomplexan parasites, such as Cryptosporidium parvum (Roberts, F.; et all Nature 1998, 393, 801; Roberts, C. W. et all J. Infect. Dis. 2002, 185 (suppl 1), S25; McConkey, G. A.; Pinney, J. W.; Westhead, D. R.; Plueckhahn, K.; Fitzpatrick, T. B.; Macheroux, P.; Kappes, B. Trends in Parasitology 2004, 20, 60).

The enzyme dehydroquinase (3-dehydroquinate dehydratase, EC 4.2.1.10) catalyzes the reversible dehydration of 3-dehydroquinic acid to form 3-dehydroshikimic acid (Scheme 1). There are two different dehydroquinases, known as type I and type II, which possess different biochemical and biophysical properties and do not show sequence similarity (Hawkins, A. R. Curr. Genet. 1987, 11, 491). These two enzymes catalyse the same reaction, but they utilize completely different mechanisms and opposite stereochemistry (Kleanthous, C.; Davis, K.; Kelly, S. M.; Cooper, A.; Harding, S. E.; Price, N. C.; Hawkins, A. R.; Coggins, J. R. Biochem. J. 1992, 282, 687).

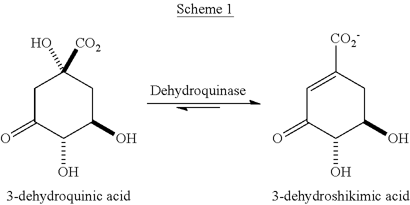

The type II enzyme (Gourley, D. G.; Coggins, J. R.; Isaacs, N. W.; Moore, J. D.; Charles, I. G.; Hawkins, A. R. J. Mol. Biol. 1994, 241, 488; Krell, T.; Pitt, A. R.; Coggins, J. R. FEBS Lett. 1995, 360, 93), may come from different sources (Mycobacterium tuberculosis, Streptomyces coelicolor, Helicobacter pylori, Aspergillus nidulans), and catalyzes the anti elimination of water.

A number of compounds with antibiotic properties have been tested in recent years, some of which are believed to inhibit the dehydroquinase of the shikimic acid pathway. For example, González-Bello, C. et al Org. Biomol. Chem., 1, 2003, p. 2075-2083 or González-Bello, C. et al Medicinal Research Reviews, Vol. 27(2), 2007, p. 177-208 discloses derivative of formula

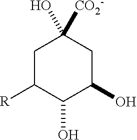

having a Kᵢ of 180 to more than 20,000 micro molar with S. coelicolor Type II Dehydroquinase. González-Bello, C. et al Org. Biomol. Chem., 1, 2003, p. 2075-2083 discloses 3-substituted derivatives of 1,4,5-Trihydroxycyclohexanecarboxylic acid, having the formula

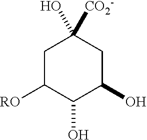

which have a Kᵢ of 180 to more than 20,000 micro molar against S. coelicolor Type II Dehydroquinase.

González-Bello, C. et al Chem Med Chem, 2008, 3, 756-770 discloses derivative of formula

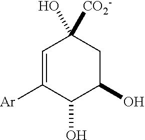

having a Kᵢ of 0.54 to more than 400 micro molar with H. pylori Type II Dehydroquinase. Compounds having the above general formula and further compounds where tested in González-Bello, C. et al Chem Med Chem, 2007, 2, p. 194-207 against S. coelicolor Type II Dehydroquinase, showing a Kᵢ between 0.13 and 33.5 micro molar.

Patent application WO 2005/009330 discloses derivatives of formula

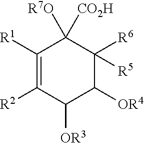

Mainly those wherein R³ and/or R⁷ are substituted benzyl groups.

Thus, there is a need to provide further compounds with antibiotic and/or antimicrobial activity.

### SUMMARY OF THE INVENTION

The present invention relates to compounds with antibiotic and/or antimicrobial activity activity, whose action is based on the effective and selective inhibition of the essential amino acids biosynthesis, particularly, by inhibition of the dehydroquinase, the third enzyme of the shikimic acid pathway.

The present invention provides compounds based on the quinic acid structure, which are effective competitive inhibitors of type II dehydroquinase, the third enzyme of the shikimic acid pathway. These compounds are significantly more potent than the described compounds of similar structure (in some cases with a Ki more than a thousand times higher). The present invention also provides procedures of obtaining of these compounds, as well as their use as antibiotics and/or antimicrobials.

Accordingly, a first aspect of the present invention is directed to a compound of formula I, its diastereoisomers, its enantiomers or its pharmaceutically acceptable salts or solvates

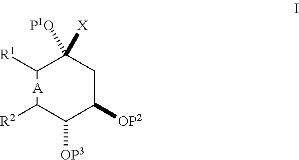

wherein,
A represents a single or double bond;
X is selected from the group consisting of —(C═O)OR^{a} and —(C═O)NR^{b}R^{c}, wherein each of R^{a}, R^{b} and R^{c} is independently selected from the group consisting of hydrogen, substituted or unsubstituted alkyl, substituted or unsubstituted alkenyl, substituted or unsubstituted alkynyl, substituted or unsubstituted aryl, substituted or unsubstituted heterocyclyl, substituted or unsubstituted arylalkyl and substituted or unsubstituted heterocyclylalkyl; or R^{b} and R^{c} together form a 5 or 6 membered heterocyclyc ring together with the nitrogen atom to which they are attached;
each of P¹, P² and P³ is independently selected from the group consisting of hydrogen, substituted or unsubstituted alkyl, substituted or unsubstituted alkenyl, substituted or unsubstituted alkynyl, substituted or unsubstituted silyl, substituted or unsubstituted arylalkyl and -(C=0)R^{a}, wherein R^{a} is as defined above; and wherein
if A is a double bond,
   then R² is selected from the group consisting of OR^{a}, —SR^{a} and NR^{b}R^{c}, wherein R^{a}, R^{b} and R^{c} are as defined above; and R¹ is hydrogen or R^{1a}, wherein R^{1a} is selected from the group consisting of substituted or unsubstituted alkyl, substituted or unsubstituted alkenyl, substituted or unsubstituted alkynyl, substituted or unsubstituted aryl, substituted or unsubstituted heterocyclyl, substituted or unsubstituted arylalkyl and substituted or unsubstituted heterocyclylalkyl, or
   R¹ and R² together form a 5-membered ring; and
if A is a single bond,
   then R² is ═O, ═S or ═NR^{b}, wherein R^{b} is as defined above; and R¹ is R^{1a}, wherein R^{1a} is a defined above.

Further aspects of the present invention are methods for the synthesis of said compounds of formula I, and intermediates thereof.

A further aspect of the present invention is a pharmaceutical composition comprising said compound of formula I and a pharmaceutically acceptable carrier.

A further aspect of the present invention is a compound of formula I as defined above, for use as a medicament.

A further aspect of the present invention is a compound of formula I as defined above, for use as an antibiotic and/or antimicrobial.

### DETAILED DESCRIPTION OF THE INVENTION

Definitions

“Alkyl” refers to a straight or branched, cyclic or acyclic hydrocarbon radical consisting of carbon and hydrogen atoms, containing no unsaturation, having 1-12, preferably one to eight, more preferably one to four carbon atoms, and which is attached to the rest of the molecule by a single bond, optionally substituted by one or more substituents selected from the group consisting of an halogen atom, an alkoxy group, a cyano group, a nitro group, a thioalkoxy group, an heterocyclylalkyl group, an heterocyclyl group or CF₃, for example, methyl, ethyl, n-propyl, i-propyl, n-butyl, t-butyl, n-pentyl, cyclopropyl, etc.

“Alkenyl” refers to a straight or branched, cyclic or acyclic hydrocarbon radical consisting of carbon and hydrogen atoms, containing at least one unsaturation, conjugated or not, having 2 to 12, preferably two to eight, more preferably two to four carbon atoms, and which is attached to the rest of the molecule by a single bond. Alkenyl radicals may be optionally substituted by one or more substituents such as a halogen atom, an alkoxy group, a cyano group, a nitro group, a thioalkoxy group, an heterocyclylalkyl group, an heterocyclyl group or CF₃, such as vinyl, allyl, butenyl (e.g. 1-butenyl, 2-butenyl, 3-butenyl), or pentenyl (e.g. 1-pentenyl, 2-pentenyl, 3-pentenyl, 4-pentenyl).

“alkynyl” refers to a straight or branched, cyclic or acyclic hydrocarbon radical consisting of carbon and hydrogen atoms, containing at least one carbon-carbon triple bond, conjugated of not, having two to twelve, preferably two to eight, more preferably two to four carbon atoms, and which is attached to the rest of the molecule by a single bond, such as —CCH, —CH₂CCH, —CCCH₃, —CH₂CCCH₃. Alkynyl radicals may be optionally substituted by one or more substituents such as a halogen atom, an alkoxy group, a cyano group, a nitro group, a thioalkoxy group, an heterocyclylalkyl group, an heterocyclyl group or CF₃.

“Aryl” refers to an aromatic hydrocarbon with 6 to 10 carbon atoms, such as phenyl or naphtyl, optionally substituted by one or more substituents selected from the group consisting of a halogen atom, an alkoxy group, a cyano group, a nitro group, an thioalkoxy group, an alkyl group or CF₃.

“Silyl” refers to trialkylsilyl species which are commonly used in organic chemistry as protecting groups, such as those disclosed in Greene, T. W.; Wuts, P. G. M. “Protective Groups in Organic Synthesis”, 3° Ed., Wiley-Interscience, New York, 1999. According to a particular embodiment, a radical of formula —SiR^{d}R^{e}R^{f} wherein R^{d}, R^{e} and R^{f} are independently selected from a substituted or unsubstituted alkyl group or a substituted or unsubstituted aryl group, for example, methyl, ethyl, t-butyl, i-propyl, phenyl, etc.

“arylalkyl” refers to a one or various aryl groups bonded to the rest of the molecule by an alkyl radical, for example, benzyl, 3-(phenyl)-propyl, etc.

“Heterocyclyl” refers to a stable 3 to 15 membered-ring constituted by carbon atoms and 1 to 5 heteroatoms selected from nitrogen, oxygen and sulphur, preferably a 4 to 8 membered-ring constituted by one or more heteroatoms, and more preferably a 5 to 6 membered-ring with one or more heretoatoms. For the purposes of this invention, heterocyclyl groups can be a monocyclic, bicyclic or tricyclic systems, that can include fused rings; and the nitrogen or sulphur atom in the heterocyclic ring can be optionally oxidized; the nitrogen atom can be optionally quaternarized; and the heterocyclyl radical can be partially or totally saturated or can be aromatic. The heterocyclic ring can be substituted by one or more substituents selected from the group consisting of a halogen atom, an alkoxy group, an alkyl group, a thioalkoxy group, a cyano group, a nitro group or CF₃. Examples of such heterocycles include, for example, furan, thiophene, pyrrole, imidazole, triazole, isothiazole, benzothiophene, benzofurane, indol, benzoimidazole, tetrahydrofuran.

“Heteroaryl” refers to a heterocyclyl group wherein at least one of the rings is aromatic.

“Alkoxy” refers to a radical of formula —O-alkyl, for example, methoxy, ethoxy, propoxy, etc.

“Thioalkoxy” refers to a radical of formula —S-alkyl, for example, thiomethoxy, thioethoxy, thiopropoxy, etc.

“Amino” refers to a radical of formula —NR^{b}R^{c} wherein R^{b} and R^{c} are as previously defined.

“Alkoxycarbonyl” refers to a radical of formula —C(═O)—O-alkyl.

“Aminocarbonyl” refers to a radical of formula —C(═O)—NR^{b}R^{c}, wherein R^{b} and R^{c} are as defined above.

“Alkylcarbonyl” refers to a radical of formula —C(═O)-alkyl.

“Heterocyclylalkyl” refers to a one or various heterocyclyl groups bonded to the rest of the molecule by an alkyl radical, for example, 2-(thienyl)ethyl, benzothiophenylmethyl, etc.

“Quinic acid” refers to (1S,3R,4S,5R)-1,3,4,5-tetrahydroxycyclohexanecarboxylic acid. Unless otherwise stated, the compounds of the invention are also meant to include compounds which differ only in the presence of one or more isotopically enriched atoms. For example, compounds having the present structures except for the replacement of a hydrogen by a deuterium or tritium, or the replacement of a carbon by a ¹³C- or ¹⁴C-enriched carbon or ¹⁵N-enriched nitrogen are within the scope of this invention.

Further, the term “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

For instance, pharmaceutically acceptable salts of compounds provided herein are synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent or in a mixture of the two. According to a particular embodiment ethyl ether, ethyl acetate, ethanol, isopropanol or acetonitrile are used as solvents. Examples of the acid addition salts include mineral acid addition salts such as, for example, hydrochloride, hydrobromide, hydroiodide, sulphate, nitrate, phosphate, and organic acid addition salts such as, for example, acetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium, ammonium, magnesium, aluminium and lithium salts, and organic alkali salts such as, for example, ethylenediamine, ethanolamine, N,N-dialkylenethanolamine, triethanolamine, glutamine and basic amino acids salts.

The compounds of the invention may be in crystalline form either as free compounds or as solvates (e.g. hydrates) and it is intended that both forms are within the scope of the present invention. Methods of solvation are generally known within the art. Suitable solvates are pharmaceutically acceptable solvates. In a particular embodiment the solvate is a hydrate.

The compounds of the present invention may include diastereoisomers and/or enantiomers depending on the presence of chiral centres or isomers depending on the presence of multiple bonds (e.g. Z, E). The single isomers, diastereoisomers, enantiomers and mixtures thereof fall within the scope of the present invention.

Compounds of Formula I

According to a particular embodiment, the compound of formula 1 is a compound of formula Ia, its diastereoisomers, its enantiomers or its pharmaceutically acceptable salts or solvates

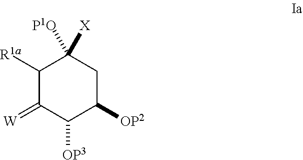

wherein X, P¹, P², P³ and R^{1a} are as defined above, and W is ═O, ═S or ═NR^{b}.

According to a particular embodiment, the compound of formula I is a compound of formula Ib, its diastereoisomers, its enantiomers or its pharmaceutically acceptable salts or solvates

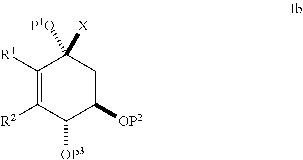

wherein,
X, P¹, P², P³ and R¹ are as defined above; and
R² is selected from the group consisting of —OR^{a}, —SR^{a} and —NR^{b}R^{c}, wherein R^{a},
R^{b} and R^{c} are as defined above.

According to a particular embodiment, the compound of formula I is a compound of formula Ic, its diastereoisomers, its enantiomers or its pharmaceutically acceptable salts or solvates

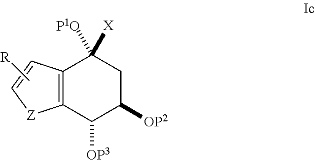

wherein,
X, P¹, P² and P³ are as defined above;
Z is selected from the group consisting of O, S, NR^{b} and ^{⊕}NR^{b}R^{c}, wherein R^{b} and R^{c} are as defined above; and
R is selected from the group consisting of a hydrogen, halogen, substituted or unsubstituted alkyl, substituted or unsubstituted alkenyl, substituted or unsubstituted alkynyl, substituted or unsubstituted aryl, substituted or unsubstituted heterocyclyl, substituted or unsubstituted arylalkyl and substituted or unsubstituted heterocyclylalkyl.

According to particular embodiment X in a compound of formula I, Ia, Ib or Ic is —CO₂H or —CO₂M, wherein M is a metal cation, preferably a metal cation of Group I of the Periodic Table, more preferably, of sodium.

According to particular embodiment X in a compound of formula I, Ia, Ib or Ic is —CO₂—C₁-C₆alkyl. According to particular embodiment X in a compound of formula I, Ia, Ib or Ic is selected form the group consisting of methoxycarbonyl, ethoxycarbonyl, propoxicarbonyl and butiroxycarbonyl.

According to a particular embodiment, R¹ in a compound of formula I, Ia or Ib is an alkyl or an alkenyl group, preferably a C₁₋₄ alkyl group or a C₁₋₄ alkenyl group.

According to a particular embodiment, R¹ in a compound of formula I, Ia or Ib is an alkyl group, preferably a C₁₋₄ alkyl group, substituted with an aryl or heteroaryl group.

According to a particular embodiment, R¹ in a compound of formula I, Ia or Ib is a radical of formula VIII

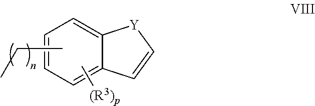

wherein
n is 1, 2, 3 or 4, preferably 1;
p is 1, 2, or 3, preferably 1;
R³ is selected from the group consisting of hydrogen, substituted or unsubstituted alkyl, substituted or unsubstituted alkenyl, substituted or unsubstituted alkynyl, substituted or unsubstituted alkoxy, substituted or unsubstituted thioalkoxy, substituted or unsubstituted aryl, substituted or unsubstituted heterocyclyl, substituted or unsubstituted arylalkyl and substituted or unsubstituted heterocyclylalkyl; and
Y is selected from the group consisting of O, S, NR^{b} and ^{⊕}NR^{b}R^{c}, wherein R^{b} and R^{c} are as defined above,

wherein the (CH₂)ₙ— moiety and R³ may be in any of the free positions.

According to a particular embodiment, R¹ in a compound of formula I, Ia or Ib is a radical of formula IX

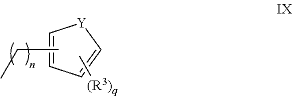

wherein
n, Y and R³ are as defined above; and
q is 1, 2, or 3, preferably 1.

According to a particular embodiment, R³ is hydrogen or alkyl, preferably C₁₋₄ alkyl.

According to a particular embodiment, Y is S or O, preferably S.

According to a particular embodiment, R¹ in a compound of formula I, Ia or Ib is substituted or unsubstituted benzyl group, preferably a radical of formula X

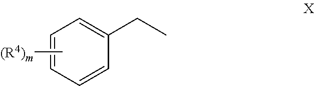

wherein
m is 0, 1, 2, 3, 4 or 5, preferably 1; and
R⁴ is selected from the group consisting of halogen, substituted or unsubstituted alkyl, substituted or unsubstituted alkenyl, substituted or unsubstituted alkynyl, substituted or unsubstituted alkoxy, substituted or unsubstituted thioalkoxy, substituted or unsubstituted aryl, substituted or unsubstituted heterocyclyl, substituted or unsubstituted arylalkyl and substituted or unsubstituted heterocyclylalkyl.

According to a particular embodiment, R⁴ is selected from the group consisting of halogen, alkyl and alkoxy. According to a further particular embodiment m is 5 and R⁴ is fluor. According to a further particular embodiment, m is 1 or 2, and R⁴ is a C₁₋₄ alkyl group or a C₁₋₄ alkoxy group.

According to a particular embodiment, R¹ in a compound of formula I, Ia or Ib is substituted or unsubstituted benzyl group, preferably a radical of formula Xa

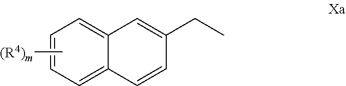

wherein
m and R⁴ are as previously defined.

According to a particular embodiment, R¹ is H in a compound of formula Ib.

According to a particular embodiment, R¹ is alkenyl in a compound of formula Ib, preferably, C₁₋₄ alkenyl, more preferably allyl.

According to a particular embodiment, R² in a compound of formula Ib is selected from the group consisting of —OR^{a}, —SR^{a} and —NR^{b}R^{c}, wherein each of R^{a}, R^{b} and R^{c} is pendently selected from the group consisting of substituted or unsubstituted alkyl, substituted or unsubstituted alkenyl, substituted or unsubstituted alkynyl, substituted or unsubstituted aryl, substituted or unsubstituted heterocyclyl, substituted or unsubstituted arylalkyl and substituted or unsubstituted heterocyclylalkyl; or R^{b} and R^{c} together form a 5 or 6 membered heterocyclyc ring together with the nitrogen atom to which they are attached.

According to a particular embodiment, R² in a compound of formula Ib is —OR^{a} or —SR^{a}, wherein R^{a} is preferably a heterocyclylalkyl group, preferably a heteroarylalkyl, more preferably a radical of formula VIII or IX as defined above, preferably a radical of formula VIII or IX wherein R³ is selected from the group consisting of alkyl, preferably C₁₋₄ alkyl and/or wherein Y is S or O, preferably S.

According to a further embodiment, R¹ and R² together form a 5-membered heteroaryl ring.

According to a particular embodiment, in a compound of formula I, Ia, Ib or Ic, at least one of P¹, P² and P³ is hydrogen, preferably P¹, P² and P³ are all hydrogen.

According to a particular embodiment, R in a compound of formula Ic is selected from the group consisting of substituted or unsubstituted alkyl, substituted or unsubstituted alkenyl, substituted or unsubstituted alkynyl and substituted or unsubstituted aryl.

According to a further particular embodiment, R in a compound of formula Ic is selected from the group consisting of substituted or unsubstituted alkyl, substituted or unsubstituted, preferably branched, alkenyl and unsubstituted aryl According to a particular embodiment, R is selected from the group consisting of substituted alkyl, substituted alkenyl, substituted alkynyl and substituted aryl, preferably a substituted alkyl or substituted alkenyl, wherein the substituent is a C₃₋₆ cycloalkyl group, preferably cyclopropyl. According to a particular embodiment, said group R is in position two of the ring.

According to a particular embodiment, Z is S.

According to a particular embodiment, W is ═S or ═O, preferably ═O.

In another more particular embodiment, the present invention relates to compounds of formula I preferably selected from:

(2R)-2-allyl-3-dehydroquinic acid,

(2S)-2-allyl-3-dehydroquinic acid,

(2R)-2-propyl-3-dehydroquinic acid,

(2R)-2-benzyl-3-dehydroquinic acid,

(2S)-2-benzyl-3-dehydroquinic acid,

(2R)-2-(4-methyl)benzyl-3-dehydroquinic acid,

(2S)-2-(4-methyl)benzyl-3-dehydroquinic acid,

(2R)-2-(4-methoxy)benzyl-3-dehydroquinic acid,

(2S)-2-(4-methoxy)benzyl-3-dehydroquinic acid,

(2R)-2-perfluorobenzyl-3-dehydroquinic acid,

(2S)-2-perfluorobenzyl-3-dehydroquinic acid,

(2R)-2-(benzo[b]thiophen-5-yl)methyl-3-dehydroquinic acid,

(2S)-2-(benzo[b]thiophen-5-yl)methyl-3-dehydroquinic acid,

Sodium (1R, 4S, 5R)-3-(benzo[b]thiophen-2-yl)methoxy-1,4,5-trihydroxycyclohex-2-en-1-carboxylate,

Sodium (1R, 4S, 5R)-3-(benzo[b]thiophen-2-yl)methoxy-2-(benzo[b]thiophen-2-yl)methyl-1,4,5-trihydroxycyclohex-2-en-1-carboxylate,

Sodium (1R, 4S, 5R)-1,4,5-trihydroxy-3-(5-methylbenzo[b]thiophen-2-yl)methoxycyclohex-2-en-1-carboxylate,

Sodium (1R, 4S, 5R)-1,4,5-trihydroxy-3-(5-methylbenzo[b]thiophen-2-yl)methoxy-2-(5-methylbenzo[b]thiophen-2-yl)methylcyclohex-2-en-1-carboxylate,

Sodium (1R, 4S, 5R)-1,4,5-trihydroxy-3-(5-methylbenzo[b]thiophen-2-yl)methoxy-2-(5-methylbenzo[b]thiophen-2-yl)methylcyclohex-2-en-1-carboxylate,

Sodium (1R, 4S, 5R)-2-allyl-3-(benzo[b]thiophen-2-yl)methoxy-1,4,5-trihydroxycylohex-2-en-1-carboxylate,

(4R, 6R, 7S)-4,6,7-trihydroxy-4,5,6,7-tetrahydrobenzo[b]thiophen-4-carboxylic acid,

(4R, 6R, 7S)-4,6,7-trihydroxy-2-methyl-4,5,6,7-tetrahydrobenzo[b]thiophen-4-carboxylic acid,

(4R, 6R, 7S)-4,6,7-trihydroxy-2-vinyl-4,5,6,7-tetrahydrobenzo[b]thiophen-4-carboxylic acid,

(4R, 6R, 7S)-4,6,7-trihydroxy-2-[(E)-prop-1-enyl]-4,5,6,7-tetrahydro-benzo[b]thiophen-4-carboxylic acid,

(4R, 6R, 7S)-4,6,7-trihydroxy-2-(1-methyl)vinyl-4,5,6,7-tetrahydrobenzo[b]thiophen-4-carboxylic acid,

(4R, 6R, 7S)-2-[(E)-2-cyclopropyl]vinyl-4,6,7-trihydroxy-4,5,6,7-tetrahydrobenzo[b]thiophen-4-carboxylic acid,

(4R, 6R, 7S)-4,6,7-trihydroxy-2-phenyl-4,5,6,7-tetrahydrobenzo[b]thiophen-4-carboxylic acid,

(4R, 6R, 7S)-2-(2-cyclopropyl)ethyl-4,6,7-trihydroxy-4,5,6,7-tetrahydro-benzo[b]thiophen-4-carboxylic acid,

(4R, 6R, 7S)-4,6,7-trihydroxy-2-isopropyl-4,5,6,7-tetrahydrobenzo[b]thiophen-4-carboxylic acid,

(4R, 6R, 7S)-2-ethyl-4,6,7-trihydroxy-4,5,6,7-tetrahydrobenzo[b]thiophen-4-carboxylic acid,

Ethyl (1R, 4S, 5R)-2-allyl-3-(benzo[b]thiophen-2-yl)methoxy-1,4,5-trihydroxycyclohex-2-en-1-carboxylate,

Sodium (1R,4S,5R)-1,4,5-tridihydroxy-3-(thien-3-yl)methoxycyclohex-2-en-1-carboxylate,

Sodium (1R, 4S, 5R)-1,4,5-trihydroxy-3-[(benzo[b]thiophen-5-yl)methoxy]cyclohex-2-en-1-carboxylate,

Sodium (1R,4S,5R)-1,4-dihydroxy-3-(thien-23-yl)methoxy-2-(thien-23-yl)methylcyclohex-2-en-1-carboxylate,

Sodium (1R, 4S, 5R)-3-[(benzo[b]thiophen-5-yl)methoxy]-2-[(benzo[b]thiophen-5-yl)methyl]-1,4-dihydroxycyclohex-2-en-1-carboxylate,

Methyl (1R,4S,5R)-3-(benzo[b]thiophen-5-yl)methoxy-2-(benzo[b]thiophen-5-yl)methyl-1,4,5-trihydroxycyclohex-2-enecarboxylate,

Methyl (1R,4S,5R)-1,4,5-tridihydroxy-3-(thien-3-yl)methoxy-2-(thien-3-yl)methylcyclohex-2-en-1-carboxylate,

Sodium (1R, 4S, 5R)-1,4,5-trihydroxy-3-(benzo[b]thiophen-5-yl)methoxy-2-(thien-2-yl)methylcyclohex-2-en-1-carboxylate,

Methyl (1R, 4S, 5R)-1,4,5-trihydroxy-3-(benzo[b]thiophen-5-yl)methoxycyclohex-2-en-1-carboxylate,

(4R, 6R, 7S)-4,6,7-trihydroxy-2-(1-phenylvinyl)-4,5,6,7-tetrahydro-benzo[b]thiophen-4-carboxylic acid,

(4R, 6R, 7S)-4,7-dihydroxy-2-styryl-4,5,6,7-tetrahydrobenzo[b]thiophen-4-carboxylic acid,

(4R, 6R, 7S)-4,6,7-trihydroxy-2-phenethyl-4,5,6,7-tetrahydroben-zo[b]thiophene-4-carboxylic acid,

(4R,6R, 7S)-4,6,7-trihydroxy-2-propyl-4,5,6,7-tetrahydroben-zo[b]thiophene-4-carboxylic acid,

(4R, 6R, 7S)-2-Ethyl-4,6,7-trihydroxy-4,5,6,7-tetrahydrobenzo[b]tiophene-4-carboxylic acid,

(4R, 6R, 7S)-2-benzyl-4,6,7-trihydroxy-2-benzyl-4,5,6,7-tetrahydro-benzo[b]thiophen-4-carboxylic acid,

(4R, 6R, 7S)-4,6,7-trihydroxy-2-phenethyl-4,5,6,7-tetrahydro-benzo[b]thiophen-4-carboxylate,

Methyl (4R, 6R, 7S)-4,6,7-trihydroxy-2-methyl-4,5,6,7-tetrahydrobenzo[b]thiophen-4-carboxylate,

Methyl (4R, 6R, 7S)-2-ethyl-4,6,7-trihydroxy-4,5,6,7-tetrahydro-benzo[b]thiophen-4-carboxylate,

Methyl (4R, 6R, 7S)-2-[(E)-2-cyclopropyl]vinyl-4,6,7-trihydroxy-4,5,6,7-tetrahydrobenzo[b]thiophen-4-carboxylate,

Methyl (4R, 6R, 7S)-2-[(E)-prop-1-enyl]-4,6,7-trihydroxy-4,5,6,7-tetrahydrobenzo[b]thiophen-4-carboxylate,

Methyl (4R, 6R, 7S)-4,6,7-trihydroxy-2-styryl-4,5,6,7-tetrahydrobenzo[b]thiophen-4-carboxylate,

Methyl (4R, 6R, 7S)-4,7-dihydroxy-2-styryl-4,5,6,7-tetrahydro-benzo[b]thiophen-4-carboxylate,

Sodium (1R,4S,5R)-1,4-trihydroxy-3-(2-naphyl)methoxycyclohex-2-en-1-carboxylate,

Sodium (1R, 4S, 5R)-1,4,5-trihydroxy-3-(naphth-2-yl)methoxy-2-(naphth-2-yl)methylcyclohex-2-en-1-carboxylate,

Sodium (1R, 4S, 5R)-1,4,5-trihydroxy-3-(thien-2-yl)methoxy-2-(benzo[b]thiophen-2-yl)methylcyclohex-2-en-1-carboxylate,

or its enantiomers or its pharmaceutically acceptable salts or solvates.

Synthesis of Compounds of Formula I

Synthesis of Compounds of Formula Ia

Another aspect of the invention relates to a procedure of obtaining compounds of formula Ia, that comprises the ring opening of lactones of formula III in acidic medium,

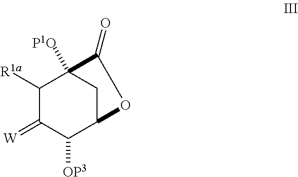

wherein, W, P¹, P³ and R^{1a} are as defined above.

The new compounds of formula III are useful intermediates for the synthesis of compounds of formula Ia. Thus, the compounds of formula III are also a further aspect of the invention. Accordingly, a further aspect of the invention relates to the preparation of compounds of formula III, comprising the C-alkylation of a compound of formula II,

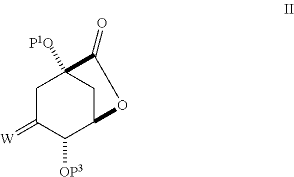

wherein W, P¹ and P³ are as defined above.

The skilled person can choose different alkylation conditions. For example, in March, J. “Advanced Organic Chemistry; Reactions Mechanism and Structure”, Wiley-Interscience, fourth ed. p. 464-473 different alkylation conditions are described. According to a particular embodiment, the alkylation is carried out in the presence of a base and of a compound R¹L, wherein L is a leaving group. The base is preferably selected from an anion of a dialkyl amine (amidure), such as lithium diisopropylamide, lithium hexamethyldisilazide, sodium hexamethyldisilazide or potassium hexamethyldisilazide. According to a particular embodiment, the leaving group is selected from iodide, bromide, chloride, tosylate, triflate or mesylate.

Compounds of formula II wherein W is ═O can be obtained by a procedure described in the article: Sánchez-Sixto, C.; Prazeres, V. F. V.; Castedo, L.; Lamb, H.; Hawkins, A. R.; González-Bello, C. J. Med. Chem. 2005, 48, 4871; and Hanessian, S.; Pan, J.; Carnell, A.; Bouchard, H.; Lesage, L. Total Synthesis of (−)-Reserpine Using the Chiron Approach J. Org. Chem. 1997, 62, 465, which are hereby entirely included by reference.

Either of the compounds of formula II, of formula III, or of formula Ia, obtained by the process described above, and wherein W is ═O, can be transformed into further compounds of formula II, of formula III, or of formula Ia, respectively, wherein W is ═S or ═NR^{b} by methods known in the art. For example, a compound wherein W is ═O can be transformed into a compound wherein W is ═S, by reaction with Lawesson's reagent (other commonly known methods are described in books, such as in March, J. “Advanced Organic Chemistry; Reactions Mechanism and Structure”, Wiley-Interscience, fourth ed. p. 893-895). A compound wherein W is ═O can be transformed into a compound wherein W is ═NR^{b}, for example, by reaction with an amine of formula H₂NR^{b} and removal of water (other commonly known methods are described in books, such as in March, J. “Advanced Organic Chemistry; Reactions Mechanism and Structure”, Wiley-Interscience, fourth ed. p. 896-898).

According to a particular embodiment, R¹ is an allyl chain. According to a further particular embodiment, R¹ is an allyl chain in a compound of formula III, which is preferably obtained by bromation of a compound of formula II, followed by an allylation reaction with a commercially available allyl tin derivative, in the presence of a catalytic amount of a radical initiator. Examples of conditions for these reactions can be found in “Bridgehead radicals in organic chemistry. An efficient construction of the ABDE ring system of the lycoctonine alkaloids”, Kraus, G. A.; Andersh, B.; Su, Q.; Shi, J. Tetrahedron Letters (1993), 34(11), 1741-4; or in “Stereoselective Reactions of a (−)-Quinic Acid-Derived Enone: Application to the Synthesis of the Core of Scyphostatin”, Murray, L. M.; O'Brien, P.; Taylor, R. J. K Organic Letters (2003), 5(11), 1943-1946. According to this embodiment, bromation is preferably carried out with a brominating agent selected from the group consisting of bromine, N-bromosuccinimide, trimethyl-phenylammonium tribromide, pyridinium bromide-perbromide, pyrrolidone hydrotri-bromide and bromine-dioxane complex. The radical initiator is preferably selected from the group consisting of tert-butyl hydroperoxide, tert-butyl perbenzoate, di(tert-butyl)peroxide, perbenzoic acid, peroxyacetic acid, 9-BBN, ZnCl₂, SmI₂, Et₃B, 2,2′-azobisisobutyronitrile or 2,2′-azobis(2-methylpropionamidine) dihydrochloride. According to a particular embodiment, the allyl tin derivative is selected from the group consisting of allyltributylstannane, tributyl(2-methylallyl)stannane, tributyl(2-phenylallyl)stannane, and tributyl(2-butylallyl)stannane.

The scope of the present invention also includes other transformations, usually functional group transformations, which transform a compound of a given formula into a different compound of the same formula. All such transformations are within the scope of the present invention.

For example, a compound of formula III wherein R^{1a} is an alkenyl group, may be transformed into compound of formula I wherein R¹ is an alkyl group, by first opening a lactone of formula III in acidic medium and then performing a catalytic hydrogenolysis over the resulting compound of formula I. Alternatively, the order of the reactions can be reversed by first performing the catalytic hydrogenolysis over a compound of formula III wherein R^{1a} is alkenyl, and then opening the lactone ring, to obtain a compound of formula I wherein R¹ is an alkyl group.

Catalytic hydrogenolysis can be carried out following known procedures (March, J. “Advanced Organic Chemistry; Reactions Mechanism and Structure”, Wiley-Interscience, fourth ed. p. 750 and 771-780), e.g. in the presence of catalyst such as palladium on carbon, palladium hydroxide, Raney Nickel, platinum, ruthenium, platinium oxide or zinc oxide.

Also, in a compound of formula Ia or III the stereochemistry of the carbon atom supporting the R¹ or R^{1a}, respectively (position 2), can be inverted by treatment with a base (for example, see Carey, F. A.; Sundberg, R. J. “Advanced Organic Chemistry. Part B: Reaction and Synthesis”, second ed. P. 1-41). For example, when R¹ or R^{1a} is allyl, R configuration at position 2 is the mayor product when allylating a compound of formula II. The present invention also provides an epimerization reaction to obtain compounds with S configuration at position 2. This epimerization reaction can be preferably carried out by treatment of compounds of formula III with a base, preferably selected from the group consisting of lithium diisopropylamide, lithium hexamethyldisilazide, sodium hexamethyldisilazide and potassium hexamethyldisilazide.

Synthesis of Compounds of Formula Ib

A further aspect of the invention relates to a procedure of obtaining compounds of formula Ib, that comprises the ring opening of lactones of formula IV in acidic or basic medium,

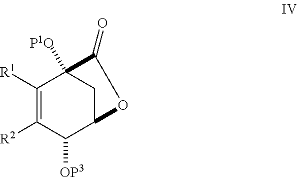

wherein, P¹, P³, P¹ and R² are as defined above.

The new compounds of formula IV are useful intermediates for the synthesis of compounds of formula Ib. Thus, the compounds of formula IV are also a further aspect of the invention. Accordingly, a further aspect of the invention relates to the preparation of compounds of formula IV, comprising: a) an O-, S- or N-alkylation reaction of compounds of formula III or of formula II; or b) a dialkylation reaction of compounds of formula II.

According to a particular embodiment, the O-, S- or N-alkylation reaction can be carried out by generation of the enol-type intermediate of a ketone, thioketone or imine of a compound of formula III in the presence of a base followed by treatment with compounds of formula R²L wherein L is a leaving group. The base is preferably selected from lithium diisopropylamidc, lithium hexamethyldisilazide, sodium hexamethyldisilazide or potassium hexamethyldisilazide and the leaving group is preferably selected from iodide, bromide, chloride, tosylate, triflate or mesylate.

According to a particular embodiment, the dialkylation reaction can be carried out by generation of the enol intermediate of a ketone, thioketone or imine of a compound of formula III in the presence of a base followed by treatment with compounds of formula R¹L wherein L is a leaving group. The base is an amine preferably selected from lithium diisopropylamide, lithium hexamethyldisilazide, sodium hexamethyldisilazide or potassium hexamethyldisilazide and the leaving group is preferably selected from iodide, bromide, chloride, tosylate, triflate or mesylate.

The O-, S- or N-alkylation reaction is favored by carring out the reaction in polar aprotic solvents such as DMF, HMPA, etc. In addition, higher O-, S- or N-alkylation ratio is obtained by using alkylating agents containing hard leaving groups, particularly oxygen-containing leaving groups, such as tosylate, mesylate or triflate or soft halides such as chloride or bromide.

On the contrary, the C-alkylation ratio is favored by using apolar or protic solvents such as Et₂O, THF, dioxane, t-BuOH, and by employing alkylating agents containing soft leaving groups such as iodide. The O-, S- or N-alkylation reaction is favored by using bromide as leaving group and N,N-dimethylformamide as reaction solvent. The C-alkylation reaction is favored by using iodide as leaving group, tetrahydrofuran as reaction solvent and in the presence of a suitable crown ether.

The skilled person can choose between O-, S- or N-alkylation and C-alkylation as mayor products through routine experimentation by introducing variations in the above mentioned factors (see pages 365-368 and 464-465 of March, J. “Advanced Organic Chemistry; Reactions Mechanism and Structure”, Wiley-Interscience, fourth ed.). Also, the reaction may provide a mixture of the O-, S- or N-alkylated product and the dialkylated product, which can be separated using standard purification techniques.

Synthesis of Compounds of Formula Ic

A further aspect of the invention relates to a procedure of obtaining compounds of formula Ic, that comprises the ring opening of lactones of formula V in acidic or basic medium,

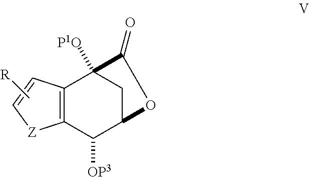

wherein, P¹, P³, R and Z are as defined above.

The new compounds of formula V are useful intermediates for the synthesis of compounds of formula Ic. Thus, the compounds of formula V are also a further aspect of the invention. Accordingly, a further aspect of the invention relates to a process for the preparation of compounds of formula V, comprising the ozonolysis of a compound of formula III, wherein R¹ is a substituted or unsubstituted allyl group, and intramolecular cyclization, in the presence of amines, phosphites or Lawesson reagent. Conditions under which these type of reactions can be performed, may be found, for example in “COMPREHENSIVE HETEROCYCLIC CHEMISTRY II, A review of the literature 1982-1995, The Structure, Reactions, Synthesis, and Uses of Heterocyclic Compounds”, Editors-in-chief Alan R. Katritzky, FRS Charles W. Rees, CBE, FRS F. V. Scriven, Volume Editor Clive W. Bird, Volume 2, “Five-membered Rings with One Heteroatom and Fused Carbocyclic Derivatives”, PERGAMON. For example, Chapter 2.03—Pyrroles and their Benzo Derivatives: Synthesis, R. J. Sundberg, University of Virginia, Charlottesville, Va., USA, pp. 119-206, describes conditions for the synthesis of compounds of formula V wherein Z is NR^{b} or ^{⊖}NR^{b}R^{c}. For example, Chapter 2.07—Furans and their Benzo Derivates: Synthesis, W. Friedrichsen, Universität Kiel, Germany. pp. 351-394, describes conditions for the synthesis of compounds of formula V wherein Z is O. For example, Chapter 2.11—Thiophenes and their Benzo Derivates: Synthesis, J. Nakayama, Saitama University, Japan. pp. 607-678, describes conditions for the synthesis of compounds of formula V wherein Z is S.

According to a particular embodiment, the process comprises the ozonolysis of a compound of formula IIIa

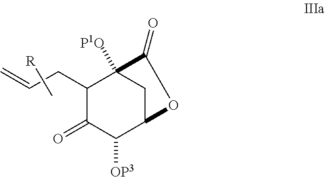

wherein, P¹, P³ and R are as defined above.

Prior to or once the cycle is formed, the resulting compound of formula Ic may undergo further transformations. Thus, if the group R is hydrogen in the compound of formula IIIa, the resulting compound of formula Ic can be further functionalized. Also, if R is different from hydrogen, said R group may be transformed.

For example, the compound of formula Ic may be optionally halogenated, and then submitted to a carbon-carbon cross-coupling reaction in order to introduce the R group. This cross-coupling reaction is typically performed in the presence of a metal catalyst, a cross-coupling reagent and a base. Preferably, the metal catalyst is selected from the group consisting of Pd(PPh₃)₄, Pd(PPh₃)₂Cl₂, Pd(OAc)₂, Pd(dppf)₂Cl₂.CH₂Cl₂, Pd₂(dba)₃, PdCl₂, Ni(PPh₃)₄ and Pd₂(dba)₃.CHCl₃. According to a particular embodiment, the base is selected from the group consisting of KO^{t}Bu, NaOAc, NaO^{t}Bu, Ba(OH)₂, N-methylmorpholine, piperidine, (ⁱPr)₂EtN, Na₂CO₃, K₂CO₃, Cs₂CO₃, K₃PO₄, Et₃N and mixtures thereof. According to a particular embodiment, said cross-coupling reagent is a boronic acid or an stannane.

Preferably, the carbon-carbon cross-coupling reaction is selected from the group consisting of Suzuki-type reaction, a Heck-type reaction, a Sonogashira-type reaction, a Negishi-type reaction or a Stille-type reaction.

For the purposes of the present invention it is understood that a Suzuki-type reaction is the cross-coupling reaction catalyzed by a palladium(0) complex between a halide or a triflate and an boronic acid or its corresponding ester, or a potassium trifluoroborate. The palladium catalysts usually employed are Pd(PPh₃)₄, Pd(PPh₃)₂Cl₂, Pd(OAc)₂, Pd₂(dba)3 o Pd₂(dba)₃.CHCl₃, optionally in the presence of phosphines or arsines, typically selected from PPh₃, P^{r}Bu₃, PCy₃ or AsPh₃. The base is usually selected from KO^{t}Bu, NaO^{t}Bu, NaOAc, Ba(OH)₂, Na₂CO₃, K₂CO₃, Cs₂CO₃, K₃PO₄, Et₃N, N-methylmorpholine or mixtures of them. This type of reactions are known to the skilled person, who can choose between a wide range of conditions (e.g., “Metal-catalyzed cross-coupling reactions”, 2nd Ed., Armin de Meijere & François Diederich, Wiley-VCH, pp 1-31 and pp 41-109)

For the purposes of the present invention it is understood that a Heck-type reaction is the reaction between an halide or a triflate with an alkene catalyzed by palladium(0). The palladium catalysts usually employed are Pd(OAc)₂, PdCl₂, Pd(PPh₃)₄ o Pd₂(dba)₃ optionally in the presence of phosphines or arsines, typically selected between PPh₃, P^{t}Bu₃, PCy₃ or AsPh₃. The base is usually selected from Na₂CO₃, K₂CO₃, NaOAc, N-methylmorpholine, K₃PO₄, Et₃N or mixtures of them. Examples of suitable Heck-type reaction conditions may be found, for example, in “Metal-catalyzed cross-coupling reactions”, 2nd Ed., Armin de Meijere & François Diederich, Wiley-VCH, pp 1-31 and pp 217-296).

For the purposes of the present invention it is understood that a Stille-type reaction is the cross-coupling reaction catalyzed by a palladium(0) complex between an halide or a triflate and an organotin compound. The palladium catalysts usually employed are Pd(dppf)₂Cl₂.CH₂Cl₂, PdCl₂, Pd(OAc)₂, Pd₂(dba)₃ or Pd(PPh₃)₄, optionally in the presence of phosphines or arsines, typically selected between PPh₃, P^{t}Bu₃, PCy₃ or AsPh₃ and in the presence of salts, such as LiCl, CsF, CuCl or CuI. Examples of suitable Heck-type reaction conditions may be found, for example, in “Metal-catalyzed cross-coupling reactions”, 2nd Ed., Armin de Meijere & François Diederich, Wiley-VCH, pp 1-31 and pp 125-155.

For the purposes of the present invention it is understood that a Sonogashira-type reaction is the cross-coupling reaction catalyzed by a palladium(0) complex and cocatalyzed by Cu(1) between an halide or a triflate and a terminal alkyne. The typical catalysts are Pd(PPh₃)₄, PdCl₂, Pd(OAc)₂ or Pd(PPh₃)₂Cl₂ optionally in the presence of phosphine or arsines, typically selected between PPh₃, P^{t}Bu₃, PCy₃ or AsPh₃. The base is usually selected from Na₂CO₃, K₂CO₃, Cs₂CO₃, Et₃N, (ⁱPr)₂EtN or mixtures of them. Examples of suitable Heck-type reaction conditions may be found, for example, in “Metal-catalyzed cross-coupling reactions”, 2nd Ed., Armin de Meijere & François Diederich, Wiley-VCH, pp 1-31 and pp 317-386.

For the purposes of the present invention it is understood that a Negishi-type reaction is the cross-coupling catalyzed by a palladium(0) or nickel(0) complex between an halide or a triflate and an organozine compound. The typical catalysts are Ni(PPh₃)₄, Pd(dppf)₂Cl₂.CH₂Cl₂, Pd(PPh₃)₂Cl₂ or Pd₂(dba)₃ optionally in the presence of phosphines or arsines, typically selected between PPh₃, P^{t}Bu₃ or PCy₃. Examples of suitable Heck-type reaction conditions may be found, for example, in “Metal-catalyzed cross-coupling reactions”, 2nd Ed., Armin de Meijere & François Diederich, Wiley-VCH, pp 1-31 and pp 815-882.

According to particular embodiment, the halogenation comprises a iodation with N-Iodosuccinimide, followed by Suzuki-type coupling between a boronic acid or its corresponding ester, for example, phenylboronic acid, (E)-prop-1-enylboronic acid, (E)-2-(cyclopropyl)vinyl-4,4,5,5-tetramethyl-1,3,2-dioxaborolane, in the presence of a palladium catalysts, for example, Pd(PPh₃)₄, Pd₂(dba)₃.CHCl₃ or Pd₂(dba)₃.

In summary, compounds of formula I can be obtained by opening of lactones of formula III, IV or V. This opening reaction can be carried out either in acidic medium, for example, in the presence of an organic acid, such as trifluoroacetic acid, p-toluensulfonic acid, camphorsulfonic acid, acetic acid, acidic ion-exchange resin; a Lewis acid or mixtures thereof. The reaction can also be carried our in basic medium, for example, in the presence of an inorganic base, such as K₂CO₃, Na₂CO₃, LiOH, NaOH, or KOH; an organic base, such as a primary amine, a secondary amine, MeONa or EtONa.

Also, any of the compounds of formula I, Ia, Ib, Ic, III, IIIa, IV or V may undergo protection-deprotection reactions using well-known procedures (Greene, T. W.; Wuts, P. G. M. “Protective Groups in Organic Synthesis”, 3° Ed., Wiley-Interscience, New York, 1999). For example, if P¹, P² and/or P³ is/are a TBS group, the deprotection will be preferably carried out by treatment with tetrabutylamonium fluoride. If P¹, P² and/or P³ is/are a benzyl group, the deprotection will be preferably performed by catalytic hydrogenolysis. If P¹, P² and/or P³ is/are an acetyl group, the deprotection will be preferably carried out by treatment with K₂CO₃ in combination with methanol. If P¹, P² and/or P³ is/are methoxyethoxymethyl ether (MEM), the deprotection will be preferably preformed by treatment with trifluoroacetic acid.

### Biological Activity

The compounds of formula I are potent competitive inhibitors of type II dehydroquinases from various bacterial sources. This enzyme acts in an essential biosynthetic route in bacteria, the shikimic acid pathway. These compounds have, in many cases, inhibition constants in the low nanomolar range and to even picomolar, which makes them, the most potent known inhibitors against any dehydroquinase.

Thus a further aspect of the invention is a pharmaceutical composition comprising a compound of formula I as defined above and a pharmaceutically acceptable carrier.

A further aspect of the invention is a compound of formula I as defined above for use as a medicament.

A further aspect of the invention is the use of a compound of formula I for the preparation of a medicament to treat tuberculosis, stomach cancer, gastritis, stomach ulcers, duodenal ulcers, or heartburn. That is, a compound of formula I for use in the treatment or prophylaxis of a disease selected from the group consisting of tuberculosis, stomach cancer, gastritis, stomach ulcers, and duodenal ulcers heartburn.

Examples of pharmaceutical compositions include any solid (tablets, pills, capsules, granules etc.) or liquid (solutions, suspensions or emulsions) compositions. Typical administration routes are oral, topical or parenteral administration. In a particular embodiment the pharmaceutical compositions are in oral form. Suitable dose forms for oral administration may be tablets and capsules and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

Suitable pharmaceutical carriers are described in “Remington's Pharmaceutical Sciences” by E. W. Martin

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

Administration of the compounds or compositions of the present invention may be by any suitable method, such as intravenous infusion, oral preparations, and intraperitoneal and intravenous administration. Oral administration is preferred because of the convenience for the patient and the chronic character of many of the diseases to be treated.

Generally an effective administered amount of a compound of the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.1 to 1000 mg/kg/day.

The compounds and compositions of this invention may be used with other drugs to provide a combination therapy. The other drugs may form part of the same composition, or be provided as a separate composition for administration at the same time or at different time.

### EXAMPLES

The Examples, which are detailed next, will have to be considered to better understanding of the present invention, which should not be interpreted as a limitation.

### Example 1

### (1S, 2R, 4S, 5R)-2-bromo-1,4-di(tert-butyldimethylsilyloxy)-3-oxocyclohexan-1,5-carbolactone (VI)

A solution of (1S, 4S, 5R)-1,4-di(tert-butyldimethylsityloxy)-3-oxocyclohexan-1,5-carbolactone (300 mg, 0.75 mmol), under inert atmosphere and at room temperature, in dry diethyl ether (20 mL) was treated with freshly made dioxane dibromide (223 mg, 0.90 mmol). The red reaction mixture was stirred at room temperature until decoloration (1.5 h), diluted with diethyl ether and washed successively with aqueous sodium bisulfate (sat.), sodium bicarbonate (sat.) and water. The organic extract was dried with Na₂SO₄ (anh.), filtered and concentrated under reduced pressure to afford α-bromo ketone VI as a white solid (356 mg, 99%). Mp: 97-100° C. [α]_{D}²⁰ −116.1° (c1.2, in CHCl₃). ¹H NMR (300 MHz, CDCl₃) δ 4.65 (dd, 1H, J=4.8 and 3.0 Hz), 4.27 (dd, 1H, J=2.1 and 0.9 Hz), 4.09 (br d, 1H, J=3.0 Hz), 3.22 (d, 1H, J=9.6 Hz), 2.44 (dddd, 1H, J=9.6, 4.8, 2.1 and 0.9 Hz), 0.94 (s, 9H), 0.90 (s, 9H), 0.23 (s, 3H), 0.18 (s, 3H), 0.15 (s, 3H) and 0.13 (s, 3H) ppm. ¹³C NMR (75 MHz, CDCl₃) δ 198.9 (C), 172.1 (C), 76.1 (C), 74.2 (CH), 71.3 (CH), 53.3 (CH), 33.6 (CH₂), 25.6 (C(CH₃)₃), 25.5 (C(CH₃)₃), 18.4 (C(CH₃)₃), 18.0 (C(CH₃)₃), −3.2 (SiCH₃), −3.3 (SiCH₃), −5.1 (SiCH₃) and −5.4 (SiCH₃) ppm. IR (KBr) 1803 (C═O) and 1731 (C═O) cm⁻¹. MS (Cl) m/z (%) 479 and 481 (MH⁺). HRMS calcd for C₁₉H₃₆O₅Br⁸¹Si₂ (MH⁺): 481.1264; found, 481.1275.

The starting material of Example 1 (1S, 4S, 5R)-1,4-di(tert-butyldimethylsilyloxy)-3-oxocyclohexan-1,5-carbolactone was obtained from (1S, 3R, 4R, 5R)-3-benciloxy-1,4-dihydroxycyclohexan-1,5-carbolactone following the three steps shown below:

Step 1: (1S, 3R, 4R, 5R)-3-benciloxy-1,4-di(tert-butyldimethylsilyloxy)cyclohexan-1,5-carbolactone. To a stirred solution of (1S, 3R, 4R, 5R)-3-benciloxy-1,4-dihydroxicyclohexan-1,5-carbolactone (1.00 g, 3.79 mmol), obtained according to Hanessian, S.; Pan, J.; Carnell, A.; Bouchard, H.; Lesage, L. Total Synthesis of (−)-Reserpine Using the Chiron Approach J. Org. Chem. 1997, 62, 465, in dry DCM (13 ml) and pyridine (1.1 mL, 13.27 mmol), under inert atmosphere at 0° C., was added tert-butyldimethylsilyl trifluorosulfonate (2.6 ml, 11.37 mmol). The resultant solution was stirred at room temperature for 12 h and then diluted with DCM and water. The aqueous layer was acidified with HCl (10%) and the organic phase was separated. The aqueous phase was extracted twice with DCM. All the combined organic extracts were dried (anh. Na₂SO₄), filtered and evaporated. The obtained residue was purified by flash chromatography eluting with 10% ethyl acetate-hexanes to yield (1S, 3R, 4R, 5R)-3-benciloxy-1,4-di(tert-butyldimethylsilyloxy)cyclohexan-1,5-carbolactone (1.81 g, 97%) as a colourless oil. [α]²⁰_{D} −14° (c1.4, in CHCl₃); ¹H NMR (250 MHz, CDCl₃) δ 7.31 (m, 5H), 4.45 (t, 1H, J 5.6), 4.42 (s, 2H), 4.11 (t, 1H, J 4.6), 3.42 (ddd, 1H, J 11.7, 6.2 and 4.1), 2.43 (d, 1H, J 11.4), 2.13-1.98 (m, 2H), 1.76 (t, 1H, J 11.9), 0.70 (s, 9H), 0.69 (s, 9H), −0.07 (s, 3H), −0.09 (s, 3H), −0.15 (s, 3H) and −0.16 (s, 3H) ppm. ¹³C NMR (63 MHz, CDCl₃) δ 176.3, 137.8, 128.3 (2×), 127.7, 127.6 (2×), 75.9, 74.1, 73.7, 71.1, 65.7, 38.1, 37.5, 25.7 (3×), 25.6 (3×), 18.1, 18.0, −2.9 (2×), −4.5 and −5.0 ppm.

Step 2: (1S, 3R, 4R, 5R)-1,4-di(tert-butyldimethylsilyloxy)-3-hydroxycyclohexan-1,5-carbolactone.cursiva A suspension of (1S, 3R, 4R, 5R)-3-benciloxy-1,4-di(tert-butyldimethylsilyloxy)cyclohexan-1,5-carbolactone (270 mg, 0.55 mmol) and 20% palladium hydroxide-on-carbon (50 mg) in methanol (15 ml) was shaken under hydrogen atmosphere at room temperature for 48 h. The mixture was filtered over Celite and the residue was washed with methanol. The filtrate and washings were evaporated under reduced pressure to yield a white solid which was purified by flash chromatography eluting with 10% ethyl acetate-hexanes to yield (1S, 3R, 4R, 5R)-1,4-di(tert-butyldimethylsilyloxy)-3-hydroxycyclohexan-1,5-carbolactone (218 mg, 99%) as white needless. Mp 107-108° C.; [α]²⁰_{D} 1° (c1.1, in CHCl₃); ¹H NMR (250 MHz, CDCl₃) δ 4.49 (t, 1H, J 5.5), 3.95 (t, 1H, J 4.8), 3.75-3.61 (m, 1H), 2.24 (d, 1H, J 11.4), 2.08 (m, 2H), 1.87 (d, 1H, J 11.6), 1.53 (t, 1H, J 12.6), 0.73 (s, 9H), 0.67 (s, 9H), −0.06 (s, 3H), −0.08 (s, 3H), −0.10 (s, 3H) and −0.11 (s, 3H) ppm; ¹³C NMR (63 MHz, CDCl₃), δ 175.9, 75.6, 73.6, 67.0, 66.1, 41.4, 37.8, 25.7 (3×), 25.5 (3×), 18.0 (2×), −2.9 (2×), −4.6 and −4.9 ppm.

Step 3: (1S, 4S, 5R)-1,4-di(tert-butyldimethylsilyloxy)-3-oxocyclohexan-1,5-carbolactone. To a stirred suspension of (1S, 3R, 4R, 5R)-1,4-di(tert-butyldimethylsilyloxy)-3-hydroxycyclohexan-1,5-carbolactone (1.24 g, 3.09 mmol) and activated powder molecular sieves 4 Å (1.24 g) in dry DCM (31 ml) was added pyridinium dichromate (1.40 g, 3.71 mmol). The resultant suspension was stirred vigorously at room temperature. After 3 h more activated powder molecular sieves 4 Å (750 mg) were added and the resultant suspension was stirred for additional 2 h. The reaction mixture was filtered over a plug of Celite and silica gel and the residue was washed with diethyl ether. The filtrate and the washings were concentrated under reduced pressure. The brown solid obtained was redisolved in hot hexane and treated with activated carbon. The black suspension was filtered over Celite and the residue was washed with hot hexane. The filtrate and the washings were concentrated and recrystallised to afford (1S, 4S, 5R)-1,4-di(tert-butyldimethylsilyloxy)-3-oxocyclohexan-1,5-carbolactone (1.17 g, 95%) as white needless. Mp 50-51° C. (hexanes); [α]²⁰_{D} −24° (c1.1, in CHCl₃); ¹H NMR (250 MHz, CDCl₃) δ 4.51 (dd, 1H, J 5.9 and 4.1), 3.81 (br d, 1H, J 4.1), 2.74 (d, 1H, J 17.6), 2.60 (ddd, 1H, J 17.6, 2.7 and 0.9), 2.50 (d, 1H, J 12.3), 2.44-2.35 (ddd, 1H, J 12.3, 5.9 and 0.9), 0.67 (s, 9H), 0.66 (s, 9H), −0.04 (s, 3H), −0.09 (s, 3H), −0.11(s, 3H), and −0.15 (s, 3H) ppm; ¹³C NMR (63 MHz, CDCl₃) δ 203.2, 175.2, 74.1, 73.1, 71.0, 50.9, 37.0, 25.5 (3×), 25.5 (3×), 18.0, 17.9, 3.1, 3.4, 4.9 and 5.3 ppm.

### Example 2

### (1R, 2R, 4S, 5R)-2-allyl-1,4-di(tert-butyldimethylsilyloxy)-3-oxocyclohexan-1,5-carbolactone (III-1)

A solution of the α-bromo ketone VI (285 mg, 0.60 mmol) in dry toluene (17 mL), under inert atmosphere, was treated allyltributyltin (0.26 mL, 0.84 mmol) and AIBN (15 mg, 0.09 mmol). The resultant reaction mixture was deoxygenated by bubbling argon through it for 30 min and then heated at 80° C. for 14 h. After cooling at room temperature, the solvent was evaporated and the crude product was purified by flash chromatography eluting with ethyl acetate-hexane (5:95) to yield the α-allyl ketone III-1 (265 mg, 99%) as beige solid. Mp: 91-94° C. [α]_{D}²⁰ −24.2° (c1.1, en CHCl₃). ¹H NMR (250 MHz, CDCl₃) δ 5.75 (m, 1H), 5.05 (m, 2H), 4.59 (dd, 1H, J=6.3 and 4.3 Hz), 3.92 (d, 1H, J=4.3 Hz), 2.89 (d, 1H, J=12.5 Hz), 2.78 (m, 1H), 2.61 (m, 1H), 2.48 (m, 1H), 2.35 (m, 1H), 0.91 (s, 9H), 0.88 (s, 9H), 0.20 (s, 3H), 0.13 (s, 3H), 0.12 (s, 3H) and 0.09 (s, 3H) ppm. ¹³C NMR (63 MHz, CDCl₃) δ 204.3 (C), 175.8 (C), 134.4 (CH), 117.4 (CH₂), 75.5 (C), 74.1 (CH), 71.5 (CH), 59.1 (CH), 32.2 (CH₂), 31.9 (CH₂), 25.6 (C(CH₃)₃), 25.5 (C(CH₃)₃), 18.3 (C(CH₃)₃), 18.0 (C(CH₃)₃), −3.2 (SiCH₃), −3.3 (SiCH₃), −4.8 (SiCH₃) and −5.4 (SiCH₃) ppm. IR (KBr): 1797 (C═O) and 1733 (C═O) cm⁻¹. MS (CI) m/z (%) 441 (MH⁺). HRMS calcd for C₂₂H₄₁O₅Si₂ (MH⁺): 441.2493; found, 441.2494. Anal. calcd for C₂₂H₄₀O₅Si₂.H₂O: C, 57.60; H, 9.23. Found: C, 57.80; H, 9.17.

### Example 3

### (2R)-2-allyl-3-dehydroquinic acid [(2R)-1a-1]

A solution of the silyl ether III-1 (250 mg, 0.57 mmol) in aqueous trifluoroacetic acid (5.7 mL, 50%) was heated at 90° C. for 3 h. After cooling at room temperature, the solvents were removed under reduced pressure. The crude residue was dissolved in water and washed with ethyl acetate (2×). The aqueous phase was lypholised and the crude product was purified by HPLC using a semipreparative column Merck LiChroCART RP-18 (10 μm, 250×10 mm) with a gradient 0-50% B (35 min) at a flow rate of 5 ml min⁻¹. The eluents for this column were: (A) water with 0.1% TFA and (B) acetonitrile with 0.1% TFA. Allyl derivative (2R)-Ia-1 (60 mg, 46%) was obtained as a white solid. Mp: 144-148° C. [α]_{D}²⁰ −30.3° (c1.0, in H₂O). ¹H NMR (400 MHz, D₂O) δ 5.82 (m, 1H), 5.10 (ddd, 1H, J=17.2, 3.2 and 1.6 Hz), 5.03 (ddd, 1H, J=10.0, 3.2 and 1.2 Hz), 4.40 (dd, 1H, J=9.6 and 1.2 Hz), 3.89 (ddd, 1H, J=11.2, 9.6 and 5.2 Hz), 3.28 (ddd, 1H, J=8.4, 4.8 and 1.2 Hz), 2.62 (m, 1H), 2.41 (dd, 1H, J=11.2 and 13.6 Hz), 2.34 (dd, 1H, J=13.6 and 5.2 Hz) and 1.98 (m, 1H) ppm. ¹³C NMR (75 MHz, D₂O) δ 211.0 (C), 179.5 (C), 138.8 (CH), 119.4 (CH₂), 83.9 (CH), 80.1 (C), 74.7 (CH), 56.3 (CH), 43.7 (CH₂) and 30.4 (CH₂) ppm. IR (KBr): 3449 (O—H), 3302 (O—H) and 1733 (C═O) cm⁻¹. MS (ESI) m/z (%) 253 (MNa⁺). HRMS calcd for C₁₀H₁₄O₆Na (MNa⁺): 253.0683; found, 253.0689.

### Example 4

### (2R)-2-propyl-3-dehydroquinic acid [(2R)-Ia-2]

A suspension of the allyl derivative (2R)-Ia-1 (15.4 mg, 0.067 mmol) and 10% palladium-on-carbon (2 mg) in methanol (0.7 mL) was stirred under hydrogen atmosphere at room temperature for 3 h. The mixture was filtered over Celite and the residue was washed with methanol. The filtrate and washings were evaporated, redissolved in water and lypholised to yield propyl derivative (2R)-Ia-2 (15.5 mg, 99%) as a light yellow solid. [α]_{D}²⁰ 29.0° (c1.7, in H₂O). ¹H NMR (250 MHz, D₂O) δ 4.31 (d, 1H, J=9.3 Hz), 3.80 (m, 1H), 3.06 (d, 1H, J=9.0 Hz), 2.37 (d, 1H, J=13.5 Hz), 2.24 (m, 1H), 1.80 (m, 1H), 1.39-0.88 (m, 3H) and 0.80 (t, 3H, J=7.3 Hz) ppm. ¹³C NMR (63 MHz, D₂O) δ 209.5 (C), 177.8 (C), 81.6 (CH), 78.8 (C), 72.6 (CH), 54.3 (CH), 41.2 (CH₂), 26.0 (CH₂), 21.2 (CH₂) and 13.8 (CH₃) ppm. IR (KBr): 3431 (O—H) and 1726 (C═O) cm¹. MS (ESI) m/z (%) 255 (MNa⁺). HRMS calcd for C₁₀H₁₆O₆Na (MH⁺): 255.0839; found, 255.0848.

### Example 5 (Epimerization)

### (1R, 2S, 4S, 5R)-2-allyl-1,4-di(tert-butyldimethylsilyloxy)-3-oxocyclohexan-1,5-carbolactone (III-2)

A stirred solution of the (2R)-2-allyl ketone III-1 (100 mg, 0.23 mmol) in dry THF (6.1 mL), under argon and at room temperature, was treated with a solution of LHMDS (345 μL, 0.35 mmol, 1.0 M in THF) and 12-crown-4 ether (4 μL, 0.023 mmol). The reaction mixture was stirred for 30 min and then diluted successively with diethyl ether and water. The organic layer was separated and the aqueous phase was extracted with diethyl ether (3×). All the combined organic extracts were dried (anh. Na₂SO₄), filtered and evaporated in vacuo. The obtained residue was purified by flash chromatography eluting with ethyl acetate-hexanes (10:90) to yield (2S)-2-allyl ketone III-2 as a light yellow oil (75 mg, 74%). [α]_{D}²⁰ −26.2° (c1.0, in CHCl₃). ¹H NMR (400 MHz, CDCl₃) α 5.92 (m, 1H), 5.05 (dq, 1H, J=17.2 and 1.6 Hz), 4.98 (m, 1H), 4.58 (dd, 1H, J=6.4 and 4.0 Hz), 4.02 (br d, 1H, J=4.0 Hz), 2.91 (dd, 1H, J=8.4 and 3.6 Hz), 2.75 (d, 1H, J=12.0 Hz), 2.62 (ddd, 1H, J=12.0, 6.4 and 0.8 Hz), 2.53 (m, 1H), 2.36 (m, 1H), 0.93 (s, 9H), 0.88 (s, 9H), 0.23 (s, 3H), 0.14 (s, 3H), 0.13 (s, 3H) and 0.09 (s, 3H) ppm. ¹³C NMR (63 MHz, CDCl₃) δ 204.9 (C), 173.9 (C), 137.0 (CH), 115.8 (CH₂), 75.7 (C), 74.0 (CH), 72.4 (CH), 60.0 (CH), 38.0 (CH₂), 27.5 (CH₂), 25.6 (2×(C(CH₃)₃), 18.1 (C(CH₃)₃), 18.0 (C(CH₃)₃), −3.3 (SiCH₃), −3.3 (SiCH₃), −4.8 (SiCH₃) and −5.2 (SiCH₃) ppm. IR (film): 1803 (C═O) and 1730 (C═O) cm⁻¹. MS (CI) m/z (%) 441 (MH⁺). HRMS calcd for C₂₂H₄₁O₅Si₂ (MH⁻): 441.2493; found, 441.2490.

### Example 6

### (2S)-2-allyl-3-dehydroquinic acid [(2S)-Ia-1]

The same experimental procedure was used as in the synthesis of acid (2R)-Ia-1 (Example 3), but using III-2 as starting material (71 mg, 0.16 mmol). Yield=25 mg (68%). Mp: 127-130° C. [α]_{D}²⁰ +17.3° (c1.8, in H₂O). ¹H NMR (400 MHz, D₂O) δ 5.67 (m, 1H), 5.15 (td, 1H, J=17.2 and 1.2 Hz), 5.09 (d, 1H, J=10.4 Hz), 4.40 (d, 1H, J=9.6 Hz), 3.90 (m, 1H), 2.81 (m, 1H) and 2.55-2.33 (m, 4H) ppm. ¹³C NMR (100 MHz, D₂O) δ 213.9 (C), 178.7 (C), 136.4 (CH), 120.8 (CH₂), 81.5 (CH), 79.4 (C), 74.0 (CH), 61.2 (CH), 38.4 (CH₂) and 36.1 (CH₂) ppm. IR (KBr): 3442 (O—H), 3415 (O—H), 1724 (C═O) and 1714 (C═O) cm ⁻¹. MS (ESI) m/z (%) 253 (MNa⁺). HRMS calcd for C₁₀H₁₄O₆Na (MNa⁺): 253.0683; found, 253.0682.

General alkylation and hydrolysis/deprotection method for Examples 7-16. A flame-dried round bottom flask was charged with (1S, 4S, 5R)-1,4-di(tert-butyldimethylsilyloxy)-3-oxocyclohexan-1,5-carbolactone (1 equivalent) and then dissolved in dry THF (0.04 M). The resultant solution was treated with 1.5 equivalents of LHMDS solution (1.0 M in THF) and 0.1 equivalents of 12-crown-4 ether. After being stirred for 20 min, a solution of the corresponding iodide (1.5 equivalents) in dry THF (0.6 M) was added. After 4 h, the reaction mixture was diluted successively with diethyl ether and ammonium chloride (sat.). The organic phase was separated and the aqueous layer was extracted three times with diethyl ether. All combined organic extracts were dried (anh. Na₂SO₄), filtered and evaporated under reduced pressure. The obtained residue was purified by flash chromatography eluting with diethyl ether-hexanes (10:90) to afford the corresponding compound of formula III as a mixture of diastereoisomers. The obtained alkyl ketones III were dissolved in 50% aqueous trifluoroacetic acid (0.1 M) and then were heated at 70° C. for 2-3 h. After cooling at room temperature, the solvents were removed under reduced pressure. The crude residue was dissolved in water and washed with ethyl acetate (2×). The aqueous phase was lypholised and the crude product was purified by HPLC using a semipreparative column Merck LiChroCART RP-18 (10 μm, 250×10 mm) with a gradient 0-50% B (35 min) at a flow rate of 5 ml min⁻¹. The eluents for this column were: (A) water with 0.1% TFA and (B) acetonitrile with 0.1% TFA. (2R)-Ia-(3-7) and (2S)-Ia-(3-7) were obtained. The spectroscopic data of the synthesized compounds using this method is indicated above:

### Example 7

### (2R)-2-benzyl-3-dehydroquinic acid [(2R)-Ia-3]

Experimental procedure using general alkylation method. Overall yield=5%. White solid. Mp: 146-148° C. ¹H NMR (400 MHz, D₂O) δ 7.39-7.24 (m, 5H), 4.34 (dd, 1H, J=9.6 and 1.2 Hz), 3.90 (ddd, 1H, J=9.6, 11.6 and 5.2 Hz), 3.54 (ddd, 1H, J=8.8, 3.6 and 1.2 Hz), 3.21 (dd, 1H, J=14.4 and 8.8 Hz), 2.52 (dd, 1H, J=14.4 and 3.6 Hz), 2.43 (dd, 1H, J=14.0 and 11.6 Hz) and 2.34 (dd, 1H, J=14.0 and 5.2 Hz) ppm. ¹³C NMR (75 MHz, D₂O) δ 208.4 (C), 177.1 (C), 140.2 (C), 129.6 (2xCH), 129.0 (2×CH), 127.0 (CH), 81.5 (CH), 78.2 (C), 72.4 (CH), 56.3 (CH), 41.3 (CH₂) and 29.7 (CH₂) ppm. IR (KBr): 3435 (O—H) and 1728 (C═O) cm⁻¹. MS (ESI) m/z (%) 303 (MNa⁺). HRMS calcd for C₁₄H₁₆O₆Na (MH): 303.0839; found, 303.0838.

### Example 8

### (2S)-2-benzyl-3-dehydroquinic acid acido [(2S)-Ia-3]

Experimental procedure using general alkylation method. Overall yield=20%. White solid. Mp: 131-134° C. ¹H NMR (400 MHz, D₂O) δ 7.39 (m, 2H), 7.33 (m, 1H), 7.25 (m, 2H), 4.61 (d, 1H, J=9.2 Hz), 3.93 (ddd, 1H, J=9.2, 11.6 and 5.2 Hz), 3.01 (m, 3H), 2.64 (dd, 1H, J=14.4 and 11.6 Hz) and 2.44 (dd, 1H, J=14.4 and 5.2 Hz) ppm. ¹³C NMR (75 MHz, D₂O) δ 210.1 (C), 175.0 (C), 136.7 (C), 128.8 (2×CH), 128.6 (2×CH), 127.0 (CH), 78.4 (CH), 76.3 (C), 70.9 (CH), 60.2 (CH), 35.1 (CH₂) and 34.6 (CH₂) ppm. IR (KBr): 3520 (O—H), 3475 (O—H), 3256 (O—H), 1719 (C═O) and 1706 (C═O) cm⁻¹. MS (ESI) m/z (%) 303 (MNa⁺). HRMS calcd for C₁₄H₁₆O₆Na (MH⁺): 303.0839; found, 303.0836.

### Example 9

### (2R)-2(4-methyl)benzyl-3-dehydroquinic acid [(2R)-Ia-4]

Experimental procedure using general alkylation method. Overall yield=5%. White solid. Mp: 105-108° C. [α]_{D}²⁰ −47.0° (c1.6, in H₂O). ¹H NMR (400 MHz, D₂O) δ 7.16 (m, 4H), 4.29 (d, 1H, J=9.6 Hz), 3.89 (m, 1H), 3.47 (m, 1H), 3.16 (dd, 1H, J=14.0 and 8.4 Hz), 2.48-2.32 (m, 3H) and 2.29 (s, 3H) ppm. ¹³C NMR (100 MHz, D₂O) δ 210.7 (C), 179.4 (C), 139.5 (C), 139.4 (C), 132.0 (2×CH), 132.0 (2×CH), 83.9 (CH), 80.5 (C), 74.8 (CH), 58.7 (CH), 43.6 (CH₂), 31.7 (CH₂) and 23.0 (CH₃) ppm. IR (KBr): 3433 (O—H) and 1726 (C═O) cm⁻¹. MS (ESI) m/z (%) 317 (MNa⁺). HRMS calcd for C₁₅H₁₈O₆Na (MNa⁺): 317.0996; found, 317.0993.

### Example 10

### (2S)-2(4-methyl)benzyl-3-dehydroquinic acid [(2S)-Ia-4]

Experimental procedure using general alkylation method. Overall yield=23%. White solid. Mp: 134-138° C. [α]_{D}²⁰ −23.2° (c1.0, in CH₃OH). ¹H NMR (400 MHz, CD₃OD) δ 7.03 (d, 2H, J=8.0 Hz), 6.98 (d, 2H, J=8.0 Hz), 4.34 (d, 1H, J=9.2 Hz), 3.80 (ddd, 1H, J=9.2, 14.4 and 5.2 Hz), 2.99 (ddd, 1H, J=11.2, 5.2 and 1.2 Hz), 2.86 (m, 2H), 2.45 (dd, 1H, J=14.4 and 11.6 Hz), 2.30 (ddd, 1H, J=1.2, 14.4 and 5.2 Hz) and 2.23 (s, 3H) ppm. ¹³C NMR (100 MHz, CD₃OD) δ 209.0 (C), 175.5 (C), 137.4 (C), 135.6 (C), 130.3 (2×CH), 129.6 (2×CH), 80.2 (CH), 77.6 (C), 73.1 (CH), 61.4 (CH), 37.1 (CH₂), 36.1 (CH₂) and 21.1 (CH₃) ppm. IR (KBr): 3484 (O—H), 3438 (O—H), 3346 (O—H) and 1718 (C═O) cm⁻¹. MS (ESI) m/z (%) 317 (MNa). HRMS calcd for C₁₅H₁₈O₆Na (MNa⁺): 317.0996; found, 317.1004.

### Example 11

### (2R)-2-(4-methoxy)benzyl-3-dehydroquinic acid [(2R)-Ia-5]

Experimental procedure using general alkylation method. Overall yield=4%. White solid. ¹H NMR (250 MHz, D₂O) δ 7.16 (d, 2H, J=8.8 Hz), 6.87 (d, 2H, J=8.8 Hz), 4.26 (d, 1H, J=9.5 Hz), 3.84 (m, 1H), 3.76 (s, 3H), 3.43 (dd, 1H, J=8.3 and 4.3 Hz), 3.10 (dd, 1H, J=14.5 and 8.5 Hz) and 2.45-2.19 (m, 3H) ppm. ¹³C NMR (75 MHz, D₂O) δ 208.7 (C), 177.6 (C), 157.8 (C), 132.9 (C), 130.8 (2×CH), 114.5 (2×CH), 81.6 (CH), 78.5 (C), 72.6 (CH), 56.6 (CH), 55.9 (CH₃), 41.4 (CH₂) and 28.9 (CH₂) ppm. IR (KBr): 3390 (O—H) and 1736 (C═O) cm⁻¹. MS (ESI) m/z (%) 333 (MNa⁺). HRMS calcd. for C₁₅H₁₈O₇Na (MNa⁺): 333.0945; found, 333.0946.

### Example 12

### (2S)-2-(4-Methoxy)benzyl-3-dehydroquinic acid [(2S)-Ia-5]

Experimental procedure using general alkylation method. Overall yield=16%. White solid. Mp: 138-143° C. [α]_{D}²⁰ −36.2° (c1.1, in MeOH). ¹H NMR (250 MHz, D₂O) δ 7.08 (d, 2H, J=8.8 Hz), 6.87 (d, 2H, J=8.8 Hz), 4.51 (d, 1H, J=9.3 Hz), 3.86 (ddd, 1H, J=14.5, 9.3 and 5.3 Hz), 3.74 (s, 3H), 2.88 (m, 3H), 2.55 (dd, 1H, J=14.5 and 11.3 Hz) and 2.37 (ddd, 1H, J=14.5, 5.3 and 1.5 Hz) ppm. ¹³C NMR (63 MHz, D₂O) δ 210.8 (C), 175.6 (C), 158.3 (C), 130.4 (2×CH), 129.8 (C), 114.8 (2×CH), 79.1 (CH), 76.9 (C), 71.6 (CH), 61.0 (CH), 55.9 (CH₃), 35.7 (CH₂) and 34.4 (CH₂) ppm. IR (KBr): 3367 (O—H), 1739 (C═O) and 1720 (C═O) cm⁻¹. MS (ESI) m/z (%) 333 (MNa⁺). HRMS calcd for C₁₅H₁₈O₇Na (MNa⁺): 333.0945; found, 333.0934.

### Example 13

### (2R)-2-perfluorobenzyl-3-dehydroquinic acid [(2R)-Ia-6]

Experimental procedure using general alkylation method. Overall yield=2%. White solid. ¹H NMR (400 MHz, D₂O) δ 4.37 (d, 1H, J=9.6 Hz), 3.90 (ddd, 1H, J=11.6, 9.6 and 5.2 Hz), 3.64 (t, 1H, J=6.6 Hz), 3.22 (dd, 1H, J=14.8 and 7.2 Hz), 2.76 (dd, 1H, J=14.8 and 6.4 Hz), 2.38 (dd, 1H, J=11.6 and 13.6 Hz) and 2.31 (dd, 1H, J=13.6 and 5.6 Hz) ppm. ¹³C NMR (75 MHz, D₂O) δ 207.9 (C), 177.0 (C), 147.8-135.9 (5×C, m), 112.6 (C, m), 81.3 (CH), 77.3 (C), 72.2 (CH), 53.1 (CH), 41.6 (CH₂) and 17.4 (CH₂) ppm. ¹⁹F NMR (282 MHz, D₂O) δ −140.3 (dd, 2F, J=21.8 and 6.1 Hz), −155.9 (t, 2F, J=21.8 Hz) and −160.1 (td, 1F, J=21.8 and 6.1 Hz) ppm. IR (KBr): 3435 (O—H) and 1730 (C═O) cm⁻¹. MS (ESI) m/z (%) 393 (MNa⁺). HRMS calcd for C₁₄H₁₁O₆F₅Na (MNa⁺): 393.0368; found, 393.0366.

### Example 14

### (2S)-2-perfluorobenzyl-3-dehydroquinic acid [(2S)-Ia-6]

Experimental procedure using general alkylation method. Overall yield=9%. White solid. Mp: 123-125° C. [α]_{D}²⁰ −27.4° (c1.3, in H₂O). ¹H NMR (400 MHz, D₂O) δ 4.64 (d, 1H, J=9.2 Hz), 3.96 (ddd, 1H, J=11.2, 9.2 and 5.2 Hz), 3.19 (dd, 1H, J=14.8 and 11.6 Hz), 3.09 (dd, 1H, J=14.8 and 4.4 Hz), 2.92 (ddd, 1H, J=11.6, 4.4 and 2.0 Hz), 2.58 (dd, 1H, J=14.4 and 11.2 Hz) and 2.47 (ddd, 1H, J=14.4, 5.2 and 2.0 Hz) ppm. ¹³C NMR (63 MHz, D₂O) δ 210.3 (C), 175.4 (C), 147.6-135.8 (5×C, m), 110.8 (C, td, J=2.8 and 18.5 Hz), 78.5 (CH), 76.7 (C), 71.2 (CH), 58.2 (CH), 35.6 (CH₂) and 22.4 (CH₂) ppm. ¹⁹F NMR (282 MHz, D₂O) δ −141.4 (dd, 2F, J=21.7 and 6.2 Hz), −154.0 (t, 2F, J=21.7 Hz) and −160.5 (td, 1F, J=21.7 and 6.2 Hz) ppm. IR (KBr): 3437 (O—H) and 1720 (C═O) cm⁻¹. MS (ESI) in/z (%) 393 (MNa⁺). HRMS calcd for C₁₄H₁₁O₆F₅Na (MNa⁺): 393.0368; found, 393.0360.

### Example 15

### (2R)-2-(benzo[b]thiophen-5-yl)methyl-3-dehydroquinic acid [(2R)-Ia-7]

Experimental procedure using general alkylation method. Overall yield=5%. [α]_{D}²⁰ −60.1° (c1.8, in CH₂OH). NMR (250 MHz, CD₂OD) δ 7.63 (m, 2H), 7.39 (d, 1H, J=5.3 Hz), 7.17 (dd, 1H, J=5.5 and 0.5 Hz), 7.13 (dd, 1H, J=8.3 and 1.8 Hz), 4.00 (d, 1H, J=9.3 Hz), 3.77 (ddd, 1H, J=11.0, 9.3 and 5.5 Hz), 3.37-3.24 (m, 2H), 2.44 (m, 1H), 2.22 (dd, 1H, J=13.5 and 11.0 Hz) and 2.14 (dd, 1H, J=13.5 and 5.5 Hz) ppm. ¹³C NMR (63 MHz, CD₃OD) δ 207.0 (C), 176.7 (C), 141.3 (C), 138.9 (C), 138.0 (C), 127.5 (CH), 127.0 (CH), 125.0 (CH), 124.7 (CH), 122.9 (CH), 82.9 (CH), 78.7 (C), 73.5 (CH), 57.9 (CH), 42.8 (CH₂) and 30.9 (CH₂) ppm. IR (KBr): 3496 (O-H), 3421 (O—H) and 1736 (C═O) cm⁻¹. MS (ESI) m/z (%) 359 (MNa⁻). HRMS calcd. for C₁₆H₁₆O₆SNa (MNa⁺): 359.0560; found, 359.0564.

### Example 16

### (2S)-2-(benzo[b]thiophen-5-yl)methyl-3-dehydroquinic acid [(2S)-Ia-7]

Experimental procedure using general alkylation method. Overall yield=19%. White solid. Mp: 152-155° C. [α]_{D}²⁰ −37.6° (c1.1, in CH₃OH). ¹H NMR (250 MHz, CD₃OD) δ 7.66 (d, 1H, J=8.3 Hz), 7.49 (s, 1H), 7.41 (d, 1H, J=5.5 Hz), 7.17 (d, 1H, J=5.5 Hz), 7.02 (dd, 1H, J=8.3 and 1.3 Hz), 4.34 (d, 1H, J=9.0 Hz), 3.76 (ddd, 1H, J=14.5, 9.0 and 5.3 Hz), 2.96 (m, 3H), 2.43 (dd, 1H, J=14.5 and 11.5 Hz) and 2.25 (ddd, 1H, J=14.5, 5.3 and 1.0 Hz) ppm. ¹³C NMR (63 MHz, CD₃OD) δ 209.0 (C), 175.4 (C), 141.5 (C), 139.6 (C), 134.8 (C), 127.9 (CH), 126.2 (CH), 124.7 (CH), 124.5 (CH), 123.5 (CH), 80.2 (CH), 77.6 (C), 73.0 (CH), 61.6 (CH), 37.1 (CH₂) and 36.4 (CH₂) ppm. IR (KBr): 3483 (O—H), 3431 (O—H), 3379 (O—H) and 1730 (C═O), 1711 and 1703 (C═O) cm⁻¹. MS (ESI) m/z (%) 359 (MNa⁺). HRMS calcd. for C₁₆H₁₆O₆SNa (MNa⁺): 359.0560; found, 359.0559.

### Example 17

### (1R, 4S, 5R)-3-(benzo[b]thiophen-2-yl)methoxy-1,4-di(tert-butyldimethylsilyloxy)cyclohex-2-en-1,5-carbolactone (IV-I) and (1R, 4S, 5R)-3-(benzo[b]thiophen-2-yl)methoxy-2-(benzo[b]thiophen-2-yl)methyl-1,4-di(tert-butyldimethylsilyloxy)cyclohex-2-en-1,5-carbolactone (IV-2)

A flame-dried round bottom flask was charged with (1R, 4S, 5R)-1,4-di(tert-butyldimethylsilyloxy)-3-oxocyclohexan-1,5-carbolactone (250 mg, 0.63 mmol) and then dissolved in dry DMF (17 mL). The resultant solution was treated with LHMDS (1.3 mL, 1.26 mmol, 1.0 M in THF) and was stirred at room temperature for 20 min. Then, a solution of 2-(bromomethyl)benzo[b]thiophene (215 mg, 0.95 mmol) in dry DMF (1.6 mL) was added. After 30 min., the reaction mixture was diluted succesively with diethyl ether and water. The organic phase was separated and the aqueous layer was extracted three times with diethyl ether. All combined organic extracts were dried (anh. Na₂SO₄), filtered and evaporated under reduced pressure. The obtained residue was purified by flash chromatography eluting with diethyl ether-hexanes (5:95) to afford O-alkyl derivative IV-1 (63 mg, 18%) and dialkyl derivative IV-2 (128 mg, 29%), both as light yellow oils.

Data for IV-1: [α]_{D}²⁰ −103.3° (c2.1, in CHCl₃). ¹H NMR (250 MHz, CDCl₃) δ 7.86-7.79 (m, 1H), 7.78-7.72 (m, 1H), 7.35 (m, 2H), 7.25 (m, 1H), 5.06 (s, 1H), 5.01 (d, 1H, J=12.0 Hz), 4.95 (d, 1H, J=12.0 Hz), 4.49 (dd, 1H, J=5.5 and 3.5 Hz), 4.19 (d, 1H, J=3.5 Hz), 2.42 (d, 1H, J=10.8 Hz), 2.34 (ddd, 1H, J=10.8, 5.5 and 1.0 Hz), 0.93 (s, 9H), 0.89 (s, 9H), 0.17 (s, 3H), 0.13 (s, 3H), 0.11 (s, 3H) and 0.10 (s, 3H) ppm. ¹³C NMR (63 MHz, CDCl₃) δ 176.1 (C), 153.2 (C), 140.3 (C), 139.1 (C), 138.9 (C), 124.5 (CH), 124.3 (CH), 123.6 (CH), 123.3 (CH), 122.4 (CH), 105.3 (CH), 75.3 (CH), 73.7 (C), 67.4 (CH), 65.2 (CH₂), 38.0 (CH₂), 25.7 (C(CH₃)₃), 25.6 (C(CH₃)₃), 18.1 (C(CH₃)₃), 18.0 (C(CH₃)₃), −3.1 (2×SiCH₃), −4.4 (SiCH₃) and −5.1 (SiCH₃) ppm. IR (film): 1803 (C═O) cm⁻¹. MS (CI) m/z (%) 547 (MH⁺). HRMS calcd for C₂₈H₄₃O₅SSi₂ (MH⁻): 547.2370; found, 547.2372.

Data for IV-2: [α]_{D}²⁰ −148.6° (c1.0, in CHCl₃). ¹H NMR (250 MHz, CDCl₃) δ 7.82-7.67 (m, 3H), 7.61 (m, 1H), 7.38-7.20 (m, 4H), 7.06 (s, 1H), 7.00 (s, 1H), 5.07 (s, 2H), 4.60 (dd, 1H, J=5.5 and 3.3 Hz), 4.50 (d, 1H, J=3.3 Hz), 3.98 (d, 1H, J=15.5 Hz), 3.83 (d, 1H, J=15.5 Hz), 2.60 (d, 1H, J=10.8 Hz), 2.48 (dd, 1H, J=10.8 and 5.8 Hz), 0.99 (s, 9H), 0.80 (s, 9H), 0.24 (s, 3H), 0.22 (s, 3H), 0.20 (s, 3H) and 0.10 (s, 3H) ppm. ¹³C NMR (63 MHz, CDCl₃) δ 175.2 (C), 148.7 (C), 144.0 (C), 140.1 (C), 139.7 (C), 139.4 (C), 139.1 (C), 128.9 (2×C), 124.4 (CH), 124.2 (CH), 123.8 (CH), 123.6 (CH), 123.1 (CH), 122.8 (CH), 122.6 (CH), 122.3 (CH), 121.9 (CH), 121.2 (CH), 74.7 (C), 74.6 (CH), 68.5 (CH₂), 67.4 (CH), 37.5 (CH₂), 25.7 (CH₂+C(CH₃)₃), 25.5 (C(CH₃)₃), 18.1 (C(CH₃)₃), 18.0 (C(CH₃)₃), −3.3 (SiCH₃), −3.4 (SiCH₃), −4.4 (SiCH₃) and −4.5 (SiCH₃) ppm. IR (film): 1799 (C═O) cm⁻¹. MS (CI) m/z (%) 693 (MH⁻). HRMS calcd for C₃₇H₄₉O₅S₂Si₂ (MH⁻): 693.2560; found, 693.2563.

### Example 18

### (1R, 4S, 5R)-3-(benzo[b]thiophen-2-yl)methoxy-1,4-dihydroxycyclohex-2-en-1,5-carbolactone (IV-3)

To a stirred solution of silylether IV-1 (42 mg, 0.077 mmol) in dry THF (1.1 mL), under argon at 0° C., was added tetrabutylammonium fluoride (0.20 mL, 0.20 mmol, ca 1.0 M in THF). After 1 h, the ice-bath was removed and the reaction mixture was stirred for another hour. The solvent was evaporated and the obtained residue was dissolved in a mixture of ethyl acetate and water. The aqueous phase was acidified with dilute HCl and the organic layer was separated. The aqueous phase was extracted with ethyl acetate (×2). Al the combined organic extracts were dried (anh. Na₂SO₄), filtered and concentrated under reduced pressure. The obtained residue was purified by flash chromatography eluting with (60:40) diethyl ether-hexanes to yield diol IV-3 (24 mg, 99%) as a colourless oil. [α]_{D}²⁰ −151.2° (c1.1, in MeOH). ¹H NMR (250 MHz, CD₃OD) δ 7.77 (m, 1H), 7.70 (m, 1H), 7.32-7.21 (m, 3H), 5.13 (s, 1H), 5.02 (br s, 2H), 4.57 (m, 1H), 4.07 (d, 1H, J=3.3 Hz) and 2.27 (m, 2H) ppm. ¹³C NMR (63 MHz, CD₃OD) δ 179.2 (C), 155.3 (C), 141.6 (C), 140.8 (C), 140.8 (C), 125.7 (CH), 125.5 (CH), 124.8 (CH), 124.6 (CH), 123.3 (CH), 105.4 (CH), 77.0 (CH), 73.0 (C), 67.6 (CH), 66.3 (CH₂) and 38.3 (CH₂) ppm. IR (KBr): 3390 (O—H) and 1765 (C═O) cm⁻¹. MS (ESI) m/z (%) 319 (MH⁻). HRMS calcd for C₁₆H₁₅O₅S (MH): 319.0635; found, 319.0634.

### Example 19

### Sodium (1R, 4S, 5R)-3-(benzo[b]thiophen-2-yl)methoxy-1,4,5-trihydroxycyclohex-2-en-1-carboxylate (Ib-1)

A solution of lactone IV-3 (28 mg, 0.088 mmol) in THF (0.8 mL) and aqueous NaOH (176 μL, 0.088 mmol, 0.5M) was stirred at room temperature for 15 min. Water was added and THF was evaporated under reduced pressure. The aqueous solution was washed with diethyl ether (×2) and liophilisated to afford O-alkyl derivative Ib-1 (31 mg, 98%) as beige solid. [α]_{D}²⁰ −52.0” (c1.3, in MeOH). ¹H NMR (250 MHz, CD₃OD) δ 7.74 (m, 1H), 7.68 (m, 1H), 7.24 (m, 3H), 5.01 (d, 1H, J=12.5 Hz), 4.94 (d, 1H, J=12.5 Hz), 4.82 (s, 1H), 3.87 (m, 2H) and 2.05 (m, 2H) ppm. ¹³C NMR (63 MHz, CD₃OD) δ 182.1 (C), 157.1 (C), 141.6 (2×C), 140.9 (C), 125.5 (CH), 125.4 (CH), 124.7 (CH), 124.1 (CH), 123.3 (CH), 103.6 (CH), 74.7 (C), 72.4 (CH), 71.6 (CH), 65.9 (OCH₂) and 37.4 (CH₂) ppm. IR (KBr): 3435 (O—H), 1664 (C═O), 1610 (C═O) and 1585 (C═O) cm⁻¹. MS (ESI) m/z (%) 359 (MH⁻). HRMS calcd for C₁₆H₁₆O₆SNa (MH⁺): 359.0560; found, 359.0560.

### Example 20

### (1R, 4S, 5R)-3-(benzo[b]thiophen-2-yl)methoxy-2-(benzo[b]thiophen-2-yl)methyl-1,4-dihydroxycyclohex-2-en-1,5-carbolactone (IV-4)

The same experimental procedure as used for compound IV-3 (example 18), was applied to silyl ether IV-2 (75 mg, 0.11 mmol) in 1.6 mL of THF and 0.29 mL of tetrabutylammonium fluoride (0.29 mmol). Yield=50 mg (98%). Light yellow oil. [α]_{D}²⁰ −228.5° (c1.0, in acetone). ¹H NMR (250 MHz, acetone-d₆) δ 7.89 (m, 1H), 7.76 (m, 2H), 7.61 (m, 1H), 7.39-7.19 (m, 5H), 7.12 (m, 1H), 5.50-5.34 (m, 2H), 4.70 (m, 2H), 4.01 (d, 1H, J=14.8 Hz), 3.82 (d, 1H, J=14.8 Hz), 2.52 (dd, 1H, J=11.0 and 2.8 Hz) and 2.42 (dd, 1H, J=11.0 and 5.8 Hz) ppm. ¹³C NMR (63 MHz, acetone-d₆) δ 177.7 (C), 149.8 (C), 146.4 (C), 143.1 (C), 142.2 (C), 141.9 (C), 141.5 (C), 141.3 (CH), 126.3 (CH), 126.2 (CH), 125.6 (CH), 125.5 (CH), 125.0 (CH), 124.9 (C), 124.6 (CH), 124.5 (CH), 124.2 (CH), 123.7 (CH), 123.3 (CH), 76.9 (CH), 74.3 (C), 67.4 (CH₂), 67.2 (CH), 39.1 (CH₂) and 26.6 (CH₂) ppm. IR (film): 3415 (O—H) and 1788 (C═O) cm⁻¹. MS (ESI) m/z (%) 487 (MNa). HRMS calcd for C₂₅H₂₀O₅S₂Na (MH⁺): 487.0644; found, 487.0644.

### Example 21

### Sodium (1R, 4S, 5R)-3-(benzo[b]thiophen-2-yl)methoxy-2-(benzo[b]thiophen-2-yl)methyl-1,4-dihydroxycyclohex-2-en-1-carboxylate (Ib-2)

The same experimental procedure as used for the synthsis of compound Ib-1 (example 19), was applied to silyl ether IV-4 (52 mg, 0.11 mmol) in 1 mL of THF and 220 μL of NaOH (aq.). Yield=54 mg (97%). Beige solid. [α]_{D}²⁰ −62.7° (c1.5, in MeOH). ¹H NMR (400 MHz, DMSO-d₆) δ 8.62 (d, 1H, J=8.0 Hz), 7.87 (m, 1H), 7.74 (m, 1H), 7.69 (m, 1H), 7.55 (m, 1H), 7.35-7.27 (m, 2H), 7.25-7.16 (m, 3H), 7.04 (s, 1H), 5.23 (br s, 1H), 5.19-5.12 (m, 3H), 4.10 (br s, 1H), 3.63 (m, 2H), 3.23 (d, 1H, J=15.2 Hz), 2.12 (dd, 1H, J=14.0 and 3.2 Hz) and 1.70 (dd, 1H, J=14.0 and 3.2 Hz) ppm. ¹³C NMR (63 MHz, DMSO-d₆) δ 177.3 (C), 150.1 (C), 146.3 (C), 142.1 (C), 139.8 (C), 139.2 (C), 139.0 (C), 138.9 (C), 124.2 (CH), 124.1 (CH), 123.6 (CH), 123.4 (CH), 122.7 (CH), 122.4 (CH), 122.2 (CH), 121.9 (CH), 121.8 (CH), 120.8 (CH), 120.5 (C), 74.2 (C), 69.8 (CH), 68.0 (CH), 64.2 (CH₂), 34.9 (CH₂) and 26.2 (CH₂) ppm. IR (KBr): 3398 (O—H) and 1601 (C═O) cm⁻¹. MS (ESI) m/z (%) 505 (MH⁺). HRMS calcd for C₂₅H₂₂O₆S₂Na (MH⁻): 505.0750; found, 505.0751.

### Example 22

### (1R, 4S, 5R)-1,4-di(tert-butyldimethylsilyloxy)-3-(5-methylbenzo[b]thiophen-2-yl)methoxycyclohex-2-en-1,5-carbolactone (IV-5) and (1R, 4S, 5R)-1,4-di(tert-butyldimethylsilyloxy)-2-(5-methylbenzo[b]thiophen-2-yl)methyl-3-(5-methylbenzo[b]thiophen-2-yl)methoxycyclohex-2-en-1,5-carbolactone (IV-6)

The experimental procedure used was the same as for compounds IV-1 and IV-2 (exmple 17) using the following: First, 200 mg of (1R, 4S, 5R)-1,4-di(tert-butyldimethylsilyloxy)-3-oxocyclohexan-1,5-carbolactone (0.50 mmol) in 13.3 mL of DMF and 1.0 mL of LHMDS (1.00 mmol) was used for the enolate generation. In the alkylation step, 180 mg of 2-(bromomethyl)-5-methylbenzo[b]thiophen (0.75 mmol) in 1.3 mL of DMF was used. Yield=62 mg (22%) of IV-5 and 99 mg (28%) of IV-6, both as light yellow oils. Data for IV-5: [α]_{D}²⁰ −120.6° (c1.0, in CHCl₃). ¹H NMR (250 MHz, CDCl₃) δ 7.70 (d, 1H, J=8.5 Hz), 7.54 (s, 1H), 7.17 (m, 2H), 5.05 (s, 1H), 4.98 (d, 1H, J=12.0 Hz), 4.92 (d, 1H, J=12.0 Hz), 4.48 (dd, 1H, J=5.3 and 3.5 Hz), 4.17 (d, 1H, J=3.5 Hz), 2.46 (s, 3H), 2.44-2.30 (m, 2H), 0.92 (s, 9H), 0.87 (s, 9H), 0.17 (s, 3H), 0.12 (s, 3H), 0.10 (s, 3H) and 0.08 (s, 3H) ppm. ¹³C NMR (63 MHz, CDCl₃) δ 176.1 (C), 153.3 (C), 139.4 (C), 138.9 (C), 137.4 (C), 134.0 (C), 126.3 (CH), 123.6 (CH), 123.0 (CH), 122.0 (CH), 105.2 (CH), 75.3 (CH), 73.7 (C), 67.4 (CH), 65.2 (OCH₂), 38.0 (CH₂), 25.7 (C(CH₃)₃), 25.6 (C(CH₃)₃), 21.3 (CH₃), 18.1 (C(CH₃)₃), 18.0 (C(CH₃)₃), −3.1 (2×SiCH₃), −4.4 (SiCH₃) and −5.2 (SiCH₃) ppm. IR (Film): 1801 (C═O) cm⁻¹. MS (CI) m/z (%) 561 (MH⁻). HRMS calcd for C₂₉H₄₅O₅SSi₂ (MH⁺): 561.2526; found, 561.2530.

Data for IV-6: [α]_{D}²⁰ −75.4° (c1.3, in CHCl₃). ¹H NMR (250 MHz, CDCl₃) δ 7.65 (d, 1H, J=8.0 Hz), 7.59 (d, 1H, J=8.0 Hz), 7.45 (s, 1H), 7.37 (s, 1H), 7.13 (d, 1H, J=8.3 Hz), 7.05 (d, 1H, J=8.3 Hz), 6.95 (s, 1H), 6.87 (s, 1H), 5.02 (s, 2H), 4.57 (d, 1H, J=5.8 and 3.3 Hz), 4.46 (d, 1H, J=3.3 Hz), 3.93 (d, 1H, J=15.5 Hz), 3.79 (d, 1H, J=15.5 Hz), 2.56 (d, 1H, J=10.8 Hz), 2.45 (m, 1H), 2.44 (s, 3H), 2.42 (s, 3H), 0.96 (s, 9H), 0.78 (s, 9H), 0.21 (s, 3H), 0.19 (s, 3H), 0.16 (s, 3H) and 0.07 (s, 3H) ppm. ¹³C NMR (63 MHz, CDCl₃) δ 175.2 (C), 148.6 (C), 144.0 (C), 140.4 (C), 139.8 (C), 139.5 (C), 137.3 (C), 136.5 (C), 133.9 (C), 133.3 (C), 129.0 (C), 126.1 (CH), 124.8 (CH), 123.6 (CH), 122.7 (CH), 122.6 (CH), 121.9 (CH), 121.5 (CH), 121.0 (CH), 74.7 (C), 74.6 (CH), 68.5 (CH₂), 67.4 (CH), 37.5 (CH₂), 25.7 (CH₂+C(CH₃)₃), 25.5 (C(CH₃)₃), 21.4 (CH₃), 21.3 (CH₃), 18.1 (C(CH₃)₃), 18.0 (C(CH₃)₃), −3.3 (SiCH₃), −3.4 (SiCH₃) and −4.5 (2×SiCH₃) ppm. IR (film): 1799 (C═O) cm⁻¹. MS (CI) m/z (%) 721 (MH⁺). HRMS calcd for C₃₉H₅₃O₅S₂Si₂ (MH⁺): 721.2873; found, 721.2878.

### Example 23

### (1R, 4S, 5R)-1,4-dihydroxy-3-(5-methylbenzo[b]thiophen-2-yl)methoxycyclohex-2-en-1,5-carbolactone (IV-7)

The experimental procedure used was the same as for compound IV-3 (example 18), but using silyl ether IV-5 as starting material (72 mg, 0.13 mmol) in 1.9 mL of THF and 0.34 mL de tetrabutylammonium fluoride (0.34 mmol). Yield=34 mg (79%). Beige solid. [α]_{D}²⁰ −135.1° (c1.4, in Me₂CO). ¹H NMR (250 MHz, CD₃OD) δ 7.63 (d, 1H, J=8.3 Hz), 7.50 (m, 1H), 7.21 (m, 1H), 7.10 (m, 1H), 5.14 (s, 1H), 5.00 (s ancho, 2H), 4.60 (m, 1H), 4.08 (d, 1H, J=3.3 Hz), 2.38 (s, 3H) and 2.28 (m, 2H) ppm. ¹³C NMR (63 MHz, CDCl₃) δ 179.2 (C), 155.3 (C), 141.1 (C), 140.8 (C), 138.8 (C), 135.3 (C), 127.3 (CH), 124.7 (CH), 124.4 (CH), 123.0 (CH), 105.4 (CH), 77.0 (CH), 73.0 (C), 67.6 (CH), 66.4 (OCH₂), 38.4 (CH₂) and 21.4 (CH₃) ppm. IR (KBr): 3444 (O—H), 3377 (O—H) and 1765 (C═O) cm⁻¹. MS (CI) m/z (%) 333 (MH⁺). HRMS calcd for C₁₇H₁₇O₃S (MH⁺): 333.0797; found, 333.0786.

### Example 24

### Sodium (1R, 4S, 5R)-1,4,5-trihydroxy-3-(5-methylbenzo[b]thiophen-2-yl)methoxycyclohex-2-en-1-carboxylate (Ib-3)

The experimental procedure used was the same as for compound Ib-1 (example 19), but using carbolactone IV-7 as starting material (21 mg, 0.063 mmol) in 0.6 mL of THF and 126 μL of NaOH (aq.). Yield=22 mg (95%). Beige solid. [α]_{D}²⁰ −41.9° (c1.5, in MeOH). ¹H NMR (250 MHz, CD₃OD) δ 7.63 (d, 1H, J=8.3 Hz), 7.50 (br s, 1H), 7.21 (s, 1H), 7.09 (dd, 1H, J=8.3 and 1.0 Hz), 5.01 (d, 1H, J=12.3 Hz), 4.94 (d, 1H, J=12.3 Hz), 4.84 (s, 1H), 3.88 (m, 2H), 2.38 (s, 3H) and 2.06 (m, 2H) ppm. ¹³C NMR (63 MHz, CD₃OD) δ 182.1 (C), 157.2 (C), 141.7 (C), 141.3 (C), 138.9 (C), 135.2 (C), 127.1 (CH), 124.6 (CH), 123.9 (CH), 122.9 (CH), 103.5 (CH), 74.7 (C), 72.4 (CH), 71.6 (CH), 66.0 (OCH₂), 37.4 (CH₂) and 21.5 (CH₃) ppm. IR (KBr): 3435 (O—H), 1649 and 1618 (C═O) cm⁻¹. MS (ESI) m/z (%) 373 (MH⁺). HRMS calcd for C₁₇H₁₈O₆SNa (MH⁺): 373.0716; found, 373.0729.

### Example 25

### (1R, 4S, 5R)-1,4-dihydroxy-3-(5-methylbenzo[b]thiophen-2-yl)methoxy-2-(5-methylbenzo[b]thiophen-2-yl)methylcyclohex-2-en-1,5-carbolactone (IV-8)

The experimental procedure used was the same as for compound IV-3 (example 18), but using silyl ether IV-6 as starting material (119 mg, 0.17 mmol) in 2.4 mL of THF and 0.44 mL de tetrabutylammonium fluoride (0.44 mmol). Yield=70 mg (84%). Beige solid. [α]_{D}²⁰ −232.4” (c1.7, in Me₂CO). ¹H NMR (250 MHz, acetone-d₆) δ 7.74 (d, 1H, J=8.3 Hz), 7.62 (d, 1H, J=8.3 Hz), 7.52 (s, 1H), 7.35 (s, 1H), 7.24 (s, 1H), 7.17 (dd, 1H, J=8.0 and 0.8 Hz), 7.06 (dd, 1H, J=8.0 and 0.8 Hz), 6.99 (s, 1H), 5.43 (d, 1H, J=12.5 Hz), 5.35 (d, 1H, J=12.5 Hz), 4.68 (m, 2H), 3.97 (d, 1H, J=14.8 Hz), 3.79 (d, 1H, J=14.8 Hz), 2.51 (d, 1H, J=12.5 Hz), 2.45-2.38 (m, 1H), 2.41 (s, 3H) and 2.38 (s, 3H) ppm. ¹³C NMR (63 MHz, acetone-d₆) δ 177.8 (C), 149.8 (C), 146.5 (C), 143.1 (C), 142.5 (C), 141.7 (C), 139.2 (C), 138.7 (C), 135.7 (C), 135.1 (C), 127.9 (CH), 126.6 (CH), 125.4 (CH), 125.0 (C), 124.5 (CH), 124.4 (CH), 123.8 (CH), 123.3 (CH), 123.1 (CH), 76.9 (CH), 74.3 (C), 67.4 (OCH₂), 67.2 (CH), 39.1 (CH2), 26.6 (CH₂) and 22.3 (2×CH₃) ppm. IR (film): 3471 (O—H), 3344 (O—H) and 1770 (C═O) cm⁻¹. MS (CI) m/z (%) 493 (MH). HRMS calcd for C₂₇H₂₅O₃S₂ (MH⁺): 493.1143; found, 493.1131.

### Example 26

### Sodium (1R, 4S, 5R)-1,4,5-trihydroxy-3-(5-methylbenzo[b]thiophen-2-yl)methoxy-2-(5-methylbenzo[b]thiophen-2-yl)methylcyclohex-2-en-1-carboxylate (Ib-4)

The experimental procedure used was the same as for compound Ib-1 (example 19), but using carbolactone IV-8 as starting material (35 mg, 0.071 mmol) in 0.65 mL of THF and 142 μL of NaOH (aq.). Yield=22 mg (95%). Beige solid. [α]_{D}²⁰ −61.3° (c1.5, in MeOH). ¹H NMR (400 MHz, DMSO-d₆) δ 8.63 (d, 1H, J=8.0 Hz), 7.73 (d, 1H, J=8.0 Hz), 7.61 (d, 1H, J=8.0 Hz), 7.45 (br s, 1H), 7.29 (br s, 1H, ArH), 7.13 (dd, 1H, J=8.0 and 1.6 Hz), 7.08 (br s, 1H), 7.01 (dd, 1H, J=8.0 and 1.6 Hz), 6.92 (s, 1H), 5.21 (br s, 1H), 5.15 (br s, 1H), 5.14 (d, 1H, J=13.2 Hz), 5.10 (d, 1H, J=13.2 Hz), 4.08 (br s, 1H), 3.64 (m, 1H), 3.60 (d, 1H, J=15.2 Hz), 3.19 (d, 1H, J=15.2 Hz), 2.38 (s, 3H), 2.35 (s, 3H), 2.11 (dd, 1H, J=14.0 and 3.2 Hz) and 1.68 (dd, 1H, J=14.0 and 3.2 Hz) ppm. ¹³C NMR (100 MHz, DMSO-d₆) δ 177.2 (C), 150.1 (C), 146.4 (C), 142.1 (C), 140.1 (C), 139.4 (C), 136.4 (C), 136.1 (C), 133.3 (C), 132.5 (C), 125.7 (CH), 124.3 (CH), 123.3 (CH), 122.2 (CH), 122.0 (CH), 121.7 (CH), 121.4 (CH), 120.6 (CH), 120.5 (C), 74.3 (C), 69.8 (CH), 68.0 (CH), 64.2 (CH₂), 34.9 (CH₂), 26.3 (CH₂), 21.0 (CH₃) and 20.9 (CH₃) ppm. IR (KBr): 3435 (O—H) and 1599 (C═O) cm⁻¹.

### Example 27

### (1R, 4S, 5R)-2-allyl-1,4-di(tert-butyldimethylsilyloxy)-3-(benzo[b]thiophen-2-yl)methoxycyclohex-2-en-1,5-carbolactone (IV-9)

To a solution of KHMDS (1.8 mL, 0.91 mmol, 0.5 M in toluene) in dry DMF (3 mL), under argon and at −78° C., a solution of (2S)-2-allyl ketone III-1 (200 mg, 0.45 mmol) in DMF (3 mL) and toluene (1.9 mL), both dry, was added. The resultant solution was stirred at this temperature for 30 min. A solution of 2-(bromomethyl)benzo[b]thiophene (206 mg, 0.91 mmol) in DMF (1.8 mL) and toluene (1.2 mL), both dry, was then added. After 1 h, water and brine were added. The aqueous phase was extracted with diethyl ether (3×2 mL). All the combined organic extracts were dried (anh. MgSO₄), filtered and concentrated. The obtained residue was purified by flash chromatography over silica gel eluting with diethyl ether/hexanes) [1° (0:100), 2°) (20:80)] to afford 118 mg (45%) of ether IV-9. Light yellow oil. [α]_{D}²⁰ −100° (c1.0, in CHCl₃). ¹H NMR (250 MHz, CDCl₃) δ 7.66 (dd, 1H, J=6.5 and 2.2 Hz), 7.59 (dd, 1H, J=6.5 and 2.5 Hz), 7.17 (m, 2H), 7.03 (s, 1H), 5.87-5.71 (m, 1H), 4.95-4.80 (m, 4H), 4.34 (dd, 1H, J=4.7 and 3.5 Hz), 4.17 (d, 1H, J=3.5 Hz), 2.96 (d, 2H, J=6.2 Hz), 2.26 (m, 2H), 0.78 (s, 9H), 0.77 (s, 9H), 0.08 (s, 3H), 0.02 (s, 3H), 0.00 (s, 3H) and −0.03 (s, 3H) ppm. ¹³C NMR (75 MHz, CDCl₃) δ 176.3 (C), 148.3 (C), 140.9 (C), 139.2 (C), 136.5 (CH), 131.2 (C), 125.1 (CH), 125.0 (CH), 124.3 (CH), 123.1 (CH), 123.0 (CH), 122.9 (C), 116.1 (CH₂), 77.3 (C), 75.4 (CH), 69.8 (CH₂), 68.1 (CH), 38.0 (CH₂), 30.1 (CH₂), 26.4 (C(CH₃)₃), 26.2 (C(CH₃)₃), 18.9 (C(CH₃)₃), 18.7 (C(CH₃)₃), −2.5 (SiCH₃), −2.6 (SiCH₃), −3.8 (SiCH₃) and −4.0 (SiCH₃) ppm. IR (film): 1799 (C—O) cm⁻¹. MS (CI) (%) 587 (MH⁺). HRMS calcd for C₃₁H₄₇O₅SSi₂ (MH⁺): 587.2683; found, 587.2682.

### Example 28

### (1R, 4S, 5R)-2-allyl-1,4-dihydroxy-3-(benzo[b]thiophen-2-yl)methoxycyclohex-2-en-1,5-carbolactone (IV-10)

The experimental procedure used was the same as for compound IV-3 (example 18), but using silyl ether IV-9 as starting material (78 mg, 0.13 mmol) in 0.9 mL of THF and 0.29 mL of tetrabutylammonium fluoride (0.29 mmol). Yield=38 mg (83%). Beige solid. Mp: 122-125° C. [α]_{D}²⁰ −143° (c1.5, in MeOH). ¹H NMR (250 MHz, CD₃OD) δ 7.76 (m, 1H), 7.69 (m, 1H, ArH), 7.26 (m, 2H), 7.22 (s, 1H), 5.78 (m, 1H), 5.22 (d, 1H, J=12.5 Hz), 5.12 (d, 1H, J=12.5 Hz), 4.97 (dq, 1H, J=17.0 and 1.7 Hz), 4.81 (m, 1H), 4.55 (m, 1H), 4.40 (d, 1H, J=3.5 Hz), 3.00 (d, 2H, J=6.5 Hz) and 2.29 (m, 2H) ppm. ¹³C NMR (63 MHz, CD₃OD) δ 178.8 (C), 148.1 (C), 142.3 (C), 141.5 (C), 140.8 (C), 137.1 (CH), 126.5 (C), 125.5 (CH), 125.3 (CH), 124.7 (CH), 123.8 (CH), 123.2 (CH), 115.5 (CH₂), 76.8 (CH), 73.8 (C), 67.2 (CH₂), 66.4 (CH₂), 38.3 (CH₂) and 29.5 (CH₂) ppm. IR (KBr): 3482 (O—H), 3369 (O—H) and 1780 (C═O) cm⁻¹. MS (ESI) m/z (%) 381 (MNa⁺). HRMS calcd for C₁₉H₁₈O₅SNa (MNa⁺): 381.0751; found, 381.0758.

### Example 29

### Sodium (1R, 4S, 5R)-2-allyl-3-(benzo[b]thiophen-2-yl)methoxy-1,4,5-tri-hydroxycyclohex-2-en-1-carboxylate (Ib-5)

The experimental procedure used was the same as for compound Ib-1 (example 19), but using carbolactone IV-10 as starting material (30 mg, 0.084 mmol) in 0.75 mL of THF and 160 μL of NaOH (aq.). Yield=33 mg (99%). Beige solid. [α]_{D}²⁰ −49° (c1.1, in H₂O). ¹H NMR (250 MHz, D₂O) δ 7.80 (m, 2H), 7.34 (m, 2H), 7.30 (s, 1H), 5.79 (m, 1H), 5.09 (d, 1H, J=11.8 Hz), 4.98 (m, 3H), 4.32 (d, 1H, J=6.7 Hz), 3.93 (m, 1H), 2.91 (dd, 1H, J=15.0 and 6.5 Hz), 2.66 (dd, 1H, J=15.0 and 6.5 Hz) and 2.05 (m, 2H) ppm. ¹³C NMR (63 MHz, D₂O) δ 180.5 (C), 150.9 (C), 140.8 (C), 140.7 (C), 139.8 (C), 137.4 (CH), 125.4 (CH), 125.2 (CH), 124.6 (CH), 123.9 (C), 123.1 (CH), 115.9 (CH2), 77.1 (C), 70.7 (CH), 70.6 (CH), 39.4 (CH₂) and 31.5 (CH₂) ppm. IR (KBr): 3427 (O—H) and 1597 (C═O) cm⁻¹. MS (ESI) m/z (%) 399 (MH⁺). HRMS calcd for C₁₉H₂₀O₆SNa (MH⁺): 399.0873; found, 399.0887.

### Example 30

### (1R, 2R, 4S, 5R)-1,4-di(tert-butyldimethylsilyloxy)-2-(2-methyl)allyl-3-oxocyclohexan-1,5-carbolactone (III-3)

A solution of the α-bromo ketone VI (500 mg, 1.04 mmol) in dry toluene (21 mL), under inert atmosphere, was treated 2-methylallyltributyltin (504 mg, 1.46 mmol) and AIBN (26 mg, 0.16 mmol). The resultant reaction mixture was deoxygenated by bubbling argon through it for 30 min and then heated at 80° C. for 14 h. After cooling at room temperature, the solvent was evaporated and the crude product was purified by flash chromatography eluting with ethyl acetate-hexane (5:95) affording 2-methylallyl ketone III-3 (457 mg, 96%) as white solid. Mp: 93-95° C. [α]_{D}²⁰ −29.1° (c1.1, in CHCl₃). ¹H NMR (250 MHz, CDCl₃) δ4.81 (br s, 1H), 4.70 (br s, 1H), 4.59 (dd, 1H, J=6.2 and 4.3 Hz), 3.92 (d, 1H, J=4.3 Hz), 2.88 (d, 1H, J=12.3 Hz), 2.75 (m, 2H), 2.49 (m, 1H), 2.37 (m, 1H), 1.75 (s, 3H), 0.91 (s, 9H), 0.89 (s, 9H), 0.22 (s, 3H), 0.14 (s, 3H), 0.12 (s, 3H) and 0.09 (s, 3H), ppm. ¹³C NMR (63 MHz, CDCl₃) δ 203.0 (C), 175.8 (C), 141.3 (C), 114.0 (CH₂), 75.5 (C), 74.1 (CH), 71.7 (CH), 57.3 (CH), 35.9 (CH₂), 32.0 (CH₂), 25.6 (2×C(CH₃)₃), 21.6 (CH₃), 18.2 (C(CH₃)₃), 18.0 (C(CH₃)₃), −3.2 (SiCH₃), −3.3 (SiCH₃), −4.7 (SiCH₃) and −5.4 (SiCH₃) ppm. IR (KBr): 1801 (C═O) and 1731 (C═O) cm⁻¹. MS (CI) m/z (%) 455 (MH⁻). HRMS calcd for C₂₃H₄₃O₅Si₂ (MH⁺): 455.2649; found, 455.2648.

### Example 31

### (4R, 6R, 7S)-4,7-di(tert-butyldimethylsilyloxy)-4,5,6,7-tetrahydrobenzo[b]thiophen-4,6-carbolactone (V-1)

A solution of allylketone III-1 (1.6 g, 3.64 mmol) in dicloromethane (125 mL) was placed into a gas-washing bottle and cooled down to −78° C. in a dry ice bath. The mixture was then flushed with argon for 5 min, oxygen for 5 min and then with ozone for 12 min. The end of the reaction was indicated by the orange coloration of the aqueous KI (5%), which is located at the exit. Afterwards, the reaction mixture was flushed for 10 min with nitrogen to remove excess ozone. The gas-washing bottle was taken from the cooling bath and the content was slowly warmed to room temperature. The solvent was evaporated under reduced pressure to afford an oil, which it was used without further purification in the subsequent cyclization reaction. The obtained residue was dissolved in dry toluene (30 mL), under argon, was treated with Lawesson's reagent (1.4 g, 3.46 mmol) and heated at 100° C. for 5 hours. After cooling to room temperature, the reaction mixture was poured into cool hexane. The obtained precipitate was filtered and washed with more hexane. The filtrate and the washings were treated with activated carbon and filtered. The solvent were concentrated and the obtained residue was crystallized from (99:1) ethanol-diethyl ether to afford tiophene V-1 (922 mg, 58%) as white needles. Mp: 133-137° C. (EtOH-Et₂O). [α]_{D}²⁰ −96.6° (c1.0, in CHCl₃). ¹H NMR (250 MHz, CDCl₃) δ 7.17 (d, 1H, J=5.2 Hz), 6.99 (d, 1H, J=5.2 Hz), 4.82 (d, 1H, J=3.2 Hz), 4.60 (dd, 1H, J=5.5 and 3.2 Hz), 2.52 (d, 1H, J=10.7 Hz), 2.44 (dd, 1H, J=10.7 and 5.5 Hz), 0.90 (s, 9H), 0.84 (s, 9H), 0.18 (s, 3H), 0.15 (s, 3H), 0.12 (s, 3H) and 0.07 (s, 3H) ppm. ¹³C NMR (63 MHz, CDCl₃) δ 175.2 (C), 141.6 (C), 134.6 (C), 125.9 (CH), 123.2 (CH), 77.3 (CH), 75.7 (C), 66.2 (CH), 37.9 (CH₂), 25.6 (2×C(CH₃)₃), 18.2 (C(CH₃)₃), 18.0 (C(CH₃)₃), −3.1 (SiCH₃), −3.2 (SiCH₃) and −4.7 (2×SiCH₃) ppm. IR (KBr): 1797 (C═O) cm⁻¹. MS (ESI) m/z (%) 441 (MH). HRMS calcd for C₂₁H₃₇O₄SSi₂ (MH⁺): 441.1946; found, 441.1964.

### Example 32

### (4R, 6R, 7S)-4,7-dihydroxy-4,5,6,7-tetrahydrobenzo[b]thiophen-4,6-carbolactone (V-2)

To a stirred solution of the silylether V-1 (70 mg, 0.16 mmol) in dry THF (2.3 mL), under argon at 0° C., was added tetrabutylammonium fluoride (0.41 mL, 0.41 mmol, ca 1.0 M en THF). After stirring for 15 min at 0° C., the ice bath was removed and the reaction mixture was stirred for 1 h. The solvent was concentrated and the obtained residue was disolved in ethyl acetate and water. The aqueous phase was acidified with HCl (10%). The organic phase was separated and the aqueous phase was extracted with ethyl acetate (×2). The combined organic extracts were dried (anh. Na₂SO₄), filtered and concentrated under reduced pressure to yield V-2 (29 mg, 85%) as a colourless oil. [α]_{D}²⁰ −112.2° (c1.0, in MeOH). ¹H NMR (250 MHz, CD₃OD) δ 7.44 (d, 1H, J=5.2 Hz), 7.11 (d, 1H, J=5.2 Hz), 4.84 (m, 3H), 4.64 (br s, 1H) and 2.52 (m, 2H) ppm. ¹³C NMR (63 MHz, CD₃OD) δ 178.4 (C), 142.2 (C), 136.4 (C), 127.7 (CH), 123.5 (CH), 79.2 (CH), 75.1 (C), 66.6 (CH) and 38.5 (CH₂) ppm. IR (KBr): 3523, 3305 (O—H), 1779 and 1753 (C═O) cm⁻¹. MS (ESI) m/z (%) 235 (MNa⁻). HRMS calcd for C₉H₈O₄SNa (MH⁺): 235.0033; found, 235.0036.

### Example 33

### (4R, 6R, 7S)-4,6,7-trihydroxy-4,5,6,7-tetrahydrobenzo[b]thiophen-4-carboxylic acid (Ic-1)

A solution of the lactone V-2 (25 mg, 0.12 mmol) in THF (0.6 mL) and aqueous LiOH (0.6 mL, 0.30 mmol, 0.5M) was stirred at room temperature for 10 min. Water was added and THF was removed under reduced pressure. The resultant aqueous solution was washed with diethyl ether (×2) and the aqueous extract was treated with Amberlite IR-120 until pH 6. The resin was filtered and washed with miliQ water. The filtrate and the washings were lyophilised to afford acid Ic-1 (25 mg, 92%) as beige solid. Mp: 128-131° C. [α]_{D}²⁰ −9.2° (c1.5, in MeOH). ¹H NMR (250 MHz, D₂O) δ 7.33 (d, 1H, J=5.0 Hz), 6.77 (d, 1H, J=5.0 Hz), 4.63 (d, 1H, J=8.3 Hz), 3.97 (m, 1H), 2.33 (dd, 1H, J=13.5 and 12.0 Hz), and 2.16 (dd, 1H, J=13.5 and 3.8 Hz) ppm. ¹³C NMR (63 MHz, D₂O) δ 178.2 (C), 143.1 (C), 135.6 (C), 128.1 (CH), 125.2 (CH), 73.6 (C), 72.8 (CH), 70.6 (CH), and 41.5 (CH₂) ppm. IR (KBr): 3437 (O—H) and 1717 (C═O) cm⁻¹. MS (ESI) m/z (%) 213 (MH⁻). HRMS calcd for C₉H₉O₄S (MH⁺): 213.0216; found, 213.0213.

### Example 34

### (4R, 6R, 7S)-4,7-di(tert-butyldimethylsilyloxy)-2-methyl-4,5,6,7-tetrahydrobenzo[b]thiophen-4,6-carbolactone (V-3)

A solution of 2-methylallyl ketone III-3 (0.47 g, 1.03 mmol) in dicloromethane (30 mL) was placed into a gas-washing bottle and cooled down to −78° C. in a dry ice bath. The mixture was then flushed with argon for 5 min, oxygen for 5 min and then with ozone for 12 min. The end of the reaction was indicated by the orange coloration of the aqueous KI (5%), which is located at the exit. Afterwards, the reaction mixture was flushed for 10 min with nitrogen to remove excess ozone. The gas-washing bottle was taken from the cooling bath and the content was slowly warming up to room temperature. The solvent was evaporated under reduced pressure to afford an oil which was used without further purification in the subsequent cyclization reaction. A small amount was purified by flash chromatography eluting with ethyl acetate-hexanes (5:95) and characterized as the ozonide intermediate. [α]_{D}²⁰ +9.7° (c1.2, en CHCl₃). ¹H NMR (250 MHz, CDCl₃) δ 4.51 (br d, 1H, J=5.8 Hz), 4.11 (br s, 1H), 2.69 (m, 1H), 2.66 (d, 1H, J=12.0 Hz), 2.36 (dd, 1H, J=13.0 and 8.8 Hz), 2.18 (dd, 1H, J=12.0 and 5.8 Hz), 2.10 (dd, 1H, J=13.0 and 5.5 Hz), 1.71 (s, 3H), 0.90 (s, 9H), 0.87 (s, 9H), 0.17 (s, 3H), 0.13 (s, 3H), 0.12 (s, 3H) and 0.08 (s, 3H) ppm. ¹³C NMR (63 MHz, CDCl₃) δ 178.6 (C), 110.2 (C), 108.6 (C), 77.9 (CH), 73.1 (C), 66.8 (CH), 48.1 (CH), 39.1 (CH₂), 33.6 (CH₂), 25.7 (2×C(CH₃)₃), 18.3 (C(CH₃)₃), 18.2 (C(CH₃)₃), 14.7 (CH₃), −3.2 (SiCH₃), −3.3 (SiCH₃), −4.7 (SiCH₃) and −5.6 (SiCH₃) ppm. IR (KBr): 1807 (C═O) cm⁻¹. MS (CI) m/z (%) 473 (MH⁺).

The obtained residue from the ozonolysis reaction was dissolved in dry toluene (30 mL) and under argon. The resultant solution was treated with Lawesson's reagent (0.33 g, 0.81 mmol) and heated at 90° C. for 3 hours. After cooling to room temperature, the reaction mixture was poured into cool hexane. The obtained precipitate was filtered and washed with more hexane. The filtrate and the washings were treated with activated carbon and filtered. The solvent were concentrated and the obtained residue was purified by flash chromatography over silica gel eluting with diethyl ether-hexanes (10:90) to afford tiophene V-3 (293 mg, 62% from III-3) as a colourless oil. [α]_{D}²⁰ −94.2° (c1.0, in CHCl₃). ¹H NMR (500 MHz, CDCl₃) δ 6.70 (br q, 1H, J=1.0 Hz), 4.81 (d, 1H, J=3.5 Hz), 4.64 (dd, 1H, J=6.0 and 3.5 Hz), 2.59 (d, 1H, J=11.0 Hz), 2.48 (dd, 1H, J=11.0 and 6.0 Hz), 2.44 (d, 3H, J=1.0 Hz), 0.98 (s, 9H), 0.92 (s, 9H), 0.25 (s, 3H), 0.21 (s, 3H), 0.18 (s, 3H) and 0.14 (s, 3H) ppm. ¹³C NMR (125 MHz, CDCl₃) δ 175.4 (C), 141.5 (C), 140.9 (C), 132.0 (C), 121.1 (CH), 77.4 (CH), 75.6 (C), 66.2 (CH), 37.9 (CH₂), 25.7 (C(CH₃)₃), 25.7 (C(CH₃)₃), 18.2 (C(CH₃)₃), 18.0 (C(CH₃)₃), 15.5 (CH₃), −3.1 (SiCH₃), −3.2 (SiCH₃) and −4.7 (2×SiCH₃) ppm. IR (KBr): 1810 (C═O) cm¹. MS (ESI) m/z (%) 455 (MH⁺). HRMS calcd for C₂₂H₃₉O₄SSi₂ (MH⁺): 455.2102; found, 455.2106.

### Example 35

### (4R, 6R, 7S)-4,7-dihydroxy-2-methyl-4,5,6,7-tetrahydrobenzo[b]thiophen-4,6-carbolactone (V-4)

The experimental procedure used was the same as for compound V-2 (example 32), but using V-3 using as starting material (175 mg, 0.38 mmol) in 5.5 mL of THF and 1 mL of tetrabutylammonium fluoride. Purification by columm chromatography eluting with diethyl ether-hexanes (70:30). Yield=61 mg (70%). Colourless oil. [α]_{D}²⁰ −100.6° (c1.0, in MeOH). ¹H NMR (250 MHz, CD₃OD) δ 6.72 (s, 1H), 4.71 (m, 2H), 3.26 (s, 1H), 2.52-2.38 (m, 6H) ppm. ¹³C NMR (63 MHz, CD₃OD) δ 178.4 (C), 142.7 (C), 142.2 (C), 133.9 (CH), 121.7 (CH), 79.2 (CH), 75.0 (C), 66.7 (CH), 38.5 (CH₂) and 15.4 (CH₃) ppm. IR (NaCl): 3444 (O—H) and 1770 (C═O) cm⁻¹. MS (ESI) m/z (%) 249 (MNa⁻). HRMS calcd for C₁₀H₁₀O₄SNa (MNa⁺): 249.0198; found, 249.0192.

### Example 36

### (4R, 6R, 7S)-4,6,7-trihydroxy-2-methyl-4,5,6,7-tetrahydrobenzo[b]thiophen-4-carboxylic acid (Ic-2)

The experimental procedure used was the same as for compound Ic-1 (example 33), but using lactone V-4 as starting material (58 mg, 0.26 mmol) in 1.3 mL of THF and 0.64 mL of LiOH (aq.). Yield=50 mg (79%). Beige solid. Mp: 92-95° C. [α]_{D}²⁰ 18.9° (c1.5, in H₂O). ¹H NMR (250 MHz, D₂O) δ 6.39 (s, 1H), 4.52 (d, 1H, J=8.0 Hz), 3.90 (ddd, 1H, J=11.5, 8.0 and 3.5 Hz), 2.26 (m, 4H) and 2.07 (dd, 1H, J=13.5 and 3.5 Hz) ppm. ¹³C NMR (63 MHz, D₂O) δ 178.4 (C), 142.5 (C), 140.0 (C), 135.5 (C), 122.7 (CH), 73.6 (C), 72.4 (CH), 70.4 (CH), 41.1 (CH₂) and 14.9 (CH₃) ppm. IR (KBr): 3434 (O—H) and 1729 (C═O) cm⁻¹. MS (ESI) m/z (%) 267 (MNa⁺). HRMS calcd for C₁₀H₁₂O₅SNa (MNa⁺): 267.0298; found, 267.0301.

### Example 37

### (4R, 6R, 7S)-4,7-di(tert-butyldimethylsilyloxy)-2-iodo-4,5,6,7-tetrahydrobenzo[b]thiophen-4,6-carbolactone (VII)

A stirred solution of thiophene V-1 (475 mg, 1.08 mmol) in dry dichloromethane (10 mL) and under argon was treated with glacial acetic acid (0.37 mL, 6.48 mmol) and N-iodosuccinimide (364 mg, 1.62 mmol). The reaction mixture was stirred at room temperature for 24 h. The solvents were removed under reduced pressure and the obtained residue was portioned in diethyl ether and 10% sodium tiosulfate. The aqueous layer was separated and the organic layer was washed with aqueous sodium bicarbonate and brine. The organic extract was dried (anh. Na₂SO₄), filtered and concentrated to afford iodide VII (569 trig, 93%) as white solid. Mp: 52-55° C. [α]_{D}²⁰ −44.9° (c1.0, in CHCl₃). ¹H NMR (250 MHz, CDCl₃) δ 7.17 (s, 1H), 4.82 (d, 1H, J=3.3 Hz), 4.65 (dd, 1H, J=5.8 and 3.3 Hz), 2.55 (d, 1H, J=11.0 Hz), 2.46 (dd, 1H, J=11.0 and 5.8 Hz), 0.98 (s, 9H), 0.91 (s, 9H), 0.25 (s, 3H), 0.21 (s, 3H), 0.18 (s, 3H) and 0.14 (s, 3H) ppm. ¹³C NMR (63 MHz, CDCl₃) δ 174.8 (C), 143.3 (C), 140.6 (C), 132.7 (CH), 77.2 (CH), 75.2 (C), 74.5 (C), 65.9 (CH), 37.6 (CH₂), 25.6 (2×C(CH₃)₃), 18.2 (C(CH₃)₃), 18.0 (C(CH₃)₃), 3.1 (SiCH₃), 3.2 (SiCH₃), 4.7 (SiCH₃) and −4.7 (SiCH₃) ppm. IR (KBr): 1801 (C═O) cm⁻¹. MS (ESI) m/z (%) 567 (MH⁻). HRMS calcd for C₂₁H₃₆O₄SSi₂I (MH⁺): 567.0986; found, 567.0919.

### Example 38

### (4R, 6R, 7S)-4,7-di(tert-butyldimethylsilyloxy)-2-vinyl-4,5,6,7-tetrahydrobenzo[b]thiophen-4,6-carbolactone (V-5)

A Shlenck tube was charged with iodide VII (150 mg, 0.26 mmol), Pd(PPh₃)₄ (31 mg, 0.03 mmol) and dry dioxane (3 mL). Anhydrous K₂CO₃ (0.72 mL, 0.79 mmol, 1.1 M) and vinyl-4,4,5,5-tetramethyl-1,3,2-dioxaborolane (61 μL, 0.40 mmol) was then added and the resultant solution was deoxygenated and heated at 100° C. for 2 h. After cooling to room temperature, the reaction mixture was filtered through a plug of Celite and the precipitate was washed with hexane. The filtrate and the washings were concentrated and the obtained residue was purified by columm chromatography over silica gel, previously neutralized with triethylamine-hexanes (5:95), using diethyl ether-hexanes (5:95) as eluent. It was obtained 83 mg of tiophene V-5 (69%) as colourless oil. [α]_{D}²⁰ +59.4° (c1.1, in CHCl₃). ¹H NMR (250 MHz, CDCl₃) δ 6.90 (s, 1H, ArH), 6.73 (dd, 1H, J=17.3 and 10.8 Hz), 5.53 (d, 1H, J=17.3 Hz), 5.15 (d, 1H, J=10.8 Hz), 4.83 (d, 1H, J=3.3 Hz), 4.66 (dd, 1H, J=5.5 and 3.3 Hz), 2.59 (d, 1H, J=11.0 Hz), 2.50 (dd, 1H, J=11.0 and 5.8 Hz), 0.98 (s, 9H), 0.93 (s, 9H), 0.26 (s, 3H), 0.23 (s, 3H), 0.20 (s, 3H) and 0.15 (s, 3H) ppm. ¹³C NMR (63 MHz, CDCl₃) δ 175.2 (C), 143.6 (C), 141.9 (C), 133.3 (C), 129.7 (CH), 121.5 (CH), 114.2 (CH₂), 77.2 (CH), 75.4 (C), 66.3 (CH), 37.7 (CH₂), 25.6 (2×C(CH₃)₃), 18.2 (C(CH₃)₃), 18.0 (C(CH₃)₃), −3.1 (SiCH₃), −3.2 (SiCH₃) and −4.7 (2×SiCH₃) ppm. IR (film): 1801 (C═O)=cm⁻¹. MS (CI) m/z (%) 467 (MH⁺). HRMS calcd for C₂₃H₃₉O₄SSi₂ (MH⁻): 467.2108; found, 467.2102.

### Example 39

### (4R, 6R, 7S)-4,7-dihydroxy-2-vinyl-4,5,6,7-tetrahydrobenzo[b]thiophen-4,6-carbolactone (V-6)

The experimental procedure used was the same as for compound V-2 (example 32), but using silyl ether V-5 as starting material (50 mg, 0.11 mmol) in 1.5 mL of THF and 0.3 mL of TBAF. Purification by columm chromatography eluting with diethyl ether-hexanes (70:30). Yield=17 mg (68%). Colourless oil. [α]_{D}²⁰ −50.9° (c1.1, in MeOH). ¹H NMR (250 MHz, CD₃OD) δ 6.94 (s, 1H), 6.76 (ddd, 1H, J=17.5, 11.0 and 0.5 Hz), 5.51 (d, 1H, J=17.5 Hz), 5.11 (dd, 1H, J=11.0 Hz), 4.74 (m, 2H) and 2.45 (m, 2H) ppm. ¹³C NMR (63 MHz, CD₃OD) δ 178.2 (C), 145.4 (C), 142.7 (C), 135.3 (C), 131.1 (CH), 122.1 (CH), 114.5 (CH₂), 79.0 (CH), 74.8 (C), 66.7 (CH) and 38.4 (CH₂) ppm. MS (CI) m/z (%) 239 (MH⁺). HRMS calcd for C₁₁H₁₁O₄S (MH): 239.0378; found, 239.0378.

### Example 40

### (4R, 6R, 7S)-4,6,7-trihydroxy-2-vinyl-4,5,6,7-tetrahydrobenzo[b]thiophen-4-carboxylic acid (Ic-3)

The experimental procedure used was the same as for compound Ic-1 (example 33), but using lactone V-6 as starting material (30 mg, 0.13 mmol) in 0.7 mL of THF and 0.3 mL of LiOH (aq.). Yield=32 mg (99%). Beige solid. ¹H NMR (250 MHz, CDCl₃) δ 6.81-6.70 (m, 2H), 5.49 (d, 1H, J=17.5 Hz), 5.46 (d, 1H, J=10.8 Hz), 4.53 (d, 1H, J=8.0 Hz), 4.03 (m, 1H), 2.39 (dd, 1H, J=13.3 and 12.0 Hz) and 2.16 (dd, 1H, J=13.3 and 3.5 Hz) ppm. ¹³C NMR (63 MHz, CDCl₃) δ 177.4 (C), 144.6 (C), 143.4 (C), 137.8 (C), 131.4 (CH), 125.1 (CH), 113.5 (CH₂), 74.1 (CH), 74.0 (C), 71.6 (CH) and 42.7 (CH₂) ppm. IR (KBr): 3400 (O—H) and 1718 (C═O) cm⁻¹. MS (ESI) m/z (%) 279 (MNa⁺). HRMS calcd for C₁₁H₁₂O₅SNa (MNa⁺): 279.0298; found, 279.0290.

### Example 41

### (4R, 6R, 7S)-4,7-di(tert-butyldimethylsilyloxy)-2-[(E)-prop-1-enyl)]-4,5,6,7-tetrahydrobenzo[b]thiophen-4,6-carbolactone (V-8)

A Shlenck tube was charged with iodide VII (100 mg, 0.18 mmol), Pd(PPh₃)₄ (20 mg, 0.02 mmol) and dry dioxane (1.8 mL). Anhydrous K₂CO₃ (0.48 mL, 0.53 mmol, 1.1 M) and (E)-prop-1-enylboronic acid (23 mg, 0.26 mmol) was then added and the resultant solution was deoxygenated and heated at 100° C. for 2 h. After cooling to room temperature, the reaction mixture was filtered through a plug of Celite and the precipitate was washed with hexane. The filtrate and the washings were concentrated and the obtained residue was purified by columm chromatography over silica gel, previously neutralized with triethylamine-hexanes (5:95), using diethyl ether-hexanes (5:95) as eluent. It was obtained 78 mg of tiophene V-8 (92%) as colourless oil. [α]_{D}²⁰ −61.9° (c1.1, in CHCl₃). ¹H NMR (250 MHz, CDCl₃) δ 6.71 (s, 1H), 6.38 (d, 1H, J=15.5 Hz), 5.97 (m, 1H), 4.74 (d, 1H, J=3.0 Hz), 4.57 (dd, 1H, J=6.0 and 3.0 Hz), 2.52 (d, 1H, J=10.8 Hz), 2.42 (dd, 1H, J=10.8 and 6.0 Hz), 1.76 (d, 3H, J=6.5 Hz), 0.91 (s, 9H), 0.85 (s, 9H), 0.19 (s, 3H), 0.15 (s, 3H), 0.12 (s, 3H) and 0.07 (s, 3H) ppm. ¹³C NMR (63 MHz, CDCl₃) δ 174.9 (C), 143.4 (C), 141.5 (C), 131.6 (C), 126.5 (CH), 123.9 (CH), 119.4 (CH), 76.2 (CH), 75.2 (C), 66.0 (CH), 37.5 (CH₂), 25.4 (2×C(CH₃)₃), 18.0 (CH₃), 17.8 (2×C(CH₃)₃), −3.3 (SiCH₃), −3.5 (SiCH₃) and −4.9 (2×SiCH₃) ppm. IR (film): 1801 (C═O) cm⁻¹. MS (CI) m/z (%) 481 (MH⁺). HRMS calcd for C₂₄H₄₁O₄SSi₂ (MH⁺): 481.2264; found, 481.2267.

### Example 42

### (4R, 6R, 7S)-4,7-dihydroxy-2-[(E)-prop-1-enyl]-4,5,6,7-tetrahydrobenzo[b]thiophen-4,6-carbolactone (V-8)

The experimental procedure used was the same as for compound V-2 (example 32), but using silyl ether V-7 as starting material (85 mg, 0.18 mmol) in 2.5 mL of THF and 0.44 mL of TBAF. Purification by columm chromatography using diethyl ether-hexano (70:30). Yield=39 mg (87%). Mp: 150-154° C. [α]_{D}²⁰ −45.6° (c1.1, in CH₃OH). ¹H NMR (250 MHz, CDCl₃) δ 6.86 (br s, 1H), 6.51 (dq, 1H, J=15.5 and 1.5 Hz), 6.09 (m, 1H), 4.81-4.75 (m, 2H), 2.49 (m, 2H) and 1.83 (dd, 3H, J=6.5 and 1.5 Hz) ppm. ¹³C NMR (63 MHz, CDCl₃) δ 178.3 (C), 145.5 (C), 142.5 (C), 133.7 (C), 127.7 (CH), 125.4 (CH), 120.3 (CH), 79.1 (CH), 74.8 (C), 66.7 (CH), 38.4 (CH₂) and 18.4 (CH₂) ppm. IR (KBr): 3305 (O—H) and 1783 and 1753 (C═O) cm⁻¹. MS (CI) m/z (%) 253 (MH⁺). HRMS calcd for C₁₂H₁₃O₄S (MH⁺): 253.0534; found, 253.0535.

### Example 43

### (4R, 6R, 7S)-4,6,7-trihydroxy-2-[(E)-prop-1-enyl]-4,5,6,7-tetrahydrobenzo[b]thiophen-4-carboxylic acid (Ic-4)

The experimental procedure used was the same as for compound Ic-1 (example 33), but using lactone V-8 as starting material (30 mg, 0.12 mmol) in 0.6 mL of THF and 0.29 mL of LiOH (aq.). Yield=31 mg (96%). White solid. [α]_{D}²⁰ −12.4° (c1.2, in CH₃OH). ¹H NMR (250 MHz, CD₃OD) δ 6.61 (s, 1H), 6.43 (d, 1H, J=15.5 Hz), 6.00 (m, 1H), 4.53 (d, 1H, J=7.0 Hz), 4.02 (m, 1H), 2.38 (m, 1H), 2.18 (m, 1H) and 1.80 (d, 1H, J=6.3 Hz) ppm. ¹³C NMR (63 MHz, CD₃OD) δ 144.5 (C), 141.2 (C), 138.4 (C), 126.5 (C), 125.8 (CH), 123.3 (CH), 73.9 (C), 73.5 (C), 71.9 (CH), 42.0 (CH₂) and 18.4 (CH₃) ppm. IR (KBr): 3399 (O—H) and 1720 (C═O) cm⁻¹. MS (ESI) m/z (%) 293 (MNa⁺). HRMS calcd for C₁₂H₁₄O₅SNa (MH⁻): 293.0454; found, 293.0453.

### Example 44

### (4R, 6R, 7S)-4,7-di(tert-butyldimethylsilyloxy)-2-(2-methyl)vinyl-4,5,6,7-tetrahydrobenzo[b]thiophen-4,6-carbolactone (V-9)

A Shlenck tube was charged with iodide VII (150 mg, 0.26 mmol), Pd(PPh₃)₄ (31 mg, 0.03 mmol) and dry dioxane (2.6 mL). Anhydrous K₂CO₃ (0.7 mL, 0.79 mmol, 1.1 M) and 4,4,5,5-tetramethyl-2-(2-methyl)vinyl-1,3,2-dioxaborolane (75 μL, 0.40 mmol) was then added and the resultant solution was deoxygenated and heated at 100° C. for 1.5 h. After cooling to room temperature, the reaction mixture was filtered through a plug of Celite and the precipitate was washed with hexane. The filtrate and the washings were concentrated and the obtained residue was purified by columm chromatography over silica gel, previously neutralized with triethylamine-hexanes (5:95), using diethyl ether-hexanes (10:90) as eluent. It was obtained 80 mg of tiophene V-9 (63%) as colourless oil. [α]_{D}²⁰ −53.7° (c1.5, in CHCl₃). ¹H NMR (300 MHz, CDCl₃) δ 6.96 (s, 1H), 5.33 (s, 1H), 4.96 (s, 1H), 4.83 (d, 1H, J=3.3 Hz), 4.66 (dd, 1H, J=6.0 and 3.3 Hz), 2.59 (d, 1H, J=10.8 Hz), 2.51 (dd, 1H, J=10.8 and 6.0 Hz), 2.10 (s, 3H), 0.99 (s, 9H), 0.93 (s, 9H), 0.26 (s, 3H), 0.23 (s, 3H), 0.20 (s, 3H) and 0.15 (s, 3H) ppm. ¹³C NMR (75 MHz, CDCl₃) δ 175.2 (C), 146.3 (C), 141.8 (C), 136.9 (C), 133.2 (C), 119.4 (CH₂), 112.0 (CH), 77.3 (CH), 75.4 (C), 66.3 (CH), 37.8 (CH₂), 25.7 (C(CH₃)₃), 25.6 (C(CH₃)₃), 21.5 (CH₃), 18.2 (C(CH₃)₃), 18.0 (C(CH₃)₃), −3.0 (SiCH₃), −3.2 (SiCH₃), −4.6 (SiCH₃) and −4.7 (SiCH₃) ppm. IR (film): 1803 (C═O) cm⁻¹. MS (ESI) m/z (%) 447 (MNa⁺−^{t}Bu).

### Example 45

### (4R, 6R, 7S)-4,7-dihydroxy-2-(2-methyl)vinyl-4,5,6,7-tetrahydrobenzo[b]thiophen-4,6-carbolactone (V-10)

The experimental procedure used was the same as for compound V-2 (example 32), but using silyl ether V-9 as starting material (110 mg, 0.23 mmol) in 3.2 mL of THF and 0.5 mL of TBAF. Purification by columm chromatography over silica gel eluting with diethyl ether-hexanes [1) 75:25, 2) 100:0]. Yield=41 mg (71%). White foam. [α]_{D}³⁰−37.0° (c1.4, in CH₃OH). ¹H NMR (250 MHz, CD₃OD) δ 7.01 (br s, 1H), 5.31 (br s, 1H), 4.93 (br s, 1H), 4.75 (m, 2H), 2.47 (m, 2H) and 2.07 (br s, 3H) ppm. ¹³C NMR (63 MHz, CD₃OD) δ 178.2 (C), 147.9 (C), 142.6 (C), 138.6 (C), 135.1 (CH), 120.0 (CH), 112.4 (CH₂), 79.0 (CH), 74.8 (C), 66.6 (CH), 38.4 (CH₂) and 21.7 (CH₃) ppm. IR (KBr): 3458 (O—H) and 1784 (C═O) cm⁻¹.

### Example 46

(4R,6R,7S)-4,6,7-trihydroxy-2-(2-methyl)vinyl-4,5,6,7-tetrahydro-benzo[b]thiophen-4-carboxylic acid (Ic-5). The experimental procedure used was the same as for compound Ic-1 (example 33), but using lactone V-10 as starting material (40 mg, 0.16 mmol) in 1.5 mL of THF and 0.8 mL of LiOH (aq.). Purification by HPLC using a semipreparative columm Merck ZORBAX ODS (212×25 mm), eluting with (20:80) acetonitrile-water and at a flow of 7 mL min⁻¹. Yield=42 mg (98%). Beige solid. [α]_{D}²⁰−1.9 (c1.4, in CH₃OH). ¹H NMR (250 MHz, D₂O) δ 6.72 (br s, 1H), 5.29 (br s, 1H), 4.92 (br s, 1H), 4.58 (d, 1H, J=8.0 Hz), 3.95 (m, 1H), 2.29 (dd, 1H, J=12.0 and 13.3 Hz), 2.13 (dd, 1H, J=3.8 and 13.3 Hz) and 1.97 (br s, 3H) ppm. ¹³C NMR (100 MHz, D₂O) δ 177.9 (C), 146.8 (C), 141.4 (C), 137.6 (C), 136.0 (C), 121.5 (CH), 111.9 (CH₂), 73.4 (C), 72.2 (CH), 70.1 (CH), 41.0 (CH₂) and 20.6 (CH₃) ppm. IR (KBr): 3438 (O—H) and 1729 (C═O) cm⁻¹. MS (ESI) m/z (%) 269 (MAT). HRMS calcd for C₁₂H₁₃O₅S (M−H⁺): 269.0478; found, 269.0469.

### Example 47

(4R,6R,7S)-4,7-di(tert-butyldimethylsilyloxy)-2-[(E)-2-cyclopropy]vinyl-4,5,6,7-tetrahydrobenzo[b]thiophen-4,6-carbolactone (V-11). A Shlenck tube was charged with iodide VII (100 mg, 0.18 mmol), Pd(PPh₃)₄ (20 mg, 0.02 mmol) and dry dioxane (1.8 mL). Anhydrous K₂CO₃ (0.5 mL, 0.53 mmol, 1.1 M) and (E)-2-(2-cyclopropyl)vinyl-4,4,5,5-tetramethyl-1,3,2-dioxaborolane (55 μL, 0.26 mmol) was then added and the resultant solution was deoxygenated and heated at 95° C. for 4 h. After cooling to room temperature, the reaction mixture was filtered through a plug of Celite and the precipitate was washed with hexane. The filtrate and the washings were concentrated and the obtained residue was was purified by columm chromatography, previously neutralized with 5% triethylamine-hexanes, eluting with diethyl ether-hexanes (5:95) to yield tiophene IXf (80 mg, 89%) as a light yellow oil. [α]_{D}²⁰−42.6° (c1.3, in CHCl₃). NMR (250 MHz, CDCl₃) δ 6.75 (s, 1H), 6.51 (d, 1H, J=15.5 Hz), 5.55 (dd, 1H, J=15.5 and 8.8 Hz), 4.80 (d, 1H, J=3.3 Hz), 4.64 (dd, 1H, J=5.5 and 3.3 Hz), 2.58 (d, 1H, J=10.8 Hz), 2.48 (dd, 1H, J=10.8 and 5.5 Hz), 1.57-1.40 (m, 1H), 0.98 (s, 9H), 0.92 (s, 9H), 0.82-0.76 (m, 2H), 0.54-0.46 (m, 2H), 0.25 (s, 3H), 0.21 (s, 3H), 0.18 (s, 3H) and 0.14 (s, 3H) ppm. ¹³C NMR (63 MHz, CDCl₃) δ 175.2 (C), 143.7 (C), 141.7 (C), 135.8 (CH), 131.4 (C), 120.6 (CH), 119.3 (CH), 77.3 (CH), 75.4 (C), 66.3 (CH), 37.8 (CH₂), 25.7 (2×C(CH₃)₃), 18.2 (C(CH₃)₃), 18.0 (C(CH₃)₃), 14.3 (CH), 7.4 (2×CH₂), −3.0 (SiCH₃), −3.2 (SiCH₃), −4.6 (SiCH₃) and −4.7 (SiCH₃) ppm. IR (film): 1801 (C═O) cm⁻¹. MS (CI) m/z (%) 507 (MH⁺). HRMS calcd for C₂₆H₄₃O₄SSi₂ (MH⁺): 507.2421; found, 507.2420.

### Example 48

(4R,6R,7S)-2-[(E)-2-cyclopropil]vinyl-4,7-dihydroxy-4,5,6,7-tetrahydrobenzo[b]thiophen-4,6-carbolactone (V-12). The experimental procedure used was the same as for compound V-2 (example 32), but using silyl ether V-11 as starting material (70 mg, 0.14 mmol) in 2 mL of THF and 0.36 mL of TBAF. Purification by columm chromatography over silica gel eluting with diethyl ether-hexanes (70:30). Yield=35 mg (92%). White solid. Mp: 154-156° C. [α]_{D}²⁰−27.0° (c1.05, in CH₃OH). ¹H NMR (250 MHz, CD₃OD) δ 6.83 (s, 1H), 6.56 (d, 1H, J=15.5 Hz), 5.61 (dd, 1H, J=15.5 and 9.0 Hz), 4.80-4.74 (m, 2H), 2.55-2.43 (m, 2H), 1.59-1.45 (m, 1H), 0.85-0.77 (m, 2H) and 0.51-0.45 (m, 2H) ppm. ¹³C NMR (63 MHz, CDCl₃) δ 178.3 (C), 145.7 (C), 142.5 (C), 137.1 (CH), 133.2 (C), 121.7 (CH), 119.8 (CH), 79.1 (CH), 74.9 (C), 66.7 (CH), 38.4 (CH₂), 15.2 (CH) and 7.8 (2×CH₂) ppm. IR (KBr): 3507 (O—H) and 1788 and 1759 (C═O) cm⁻¹. MS (CI) m/z (%) 279 (MH⁺). HRMS calcd for C₁₄H₁₅O₄S (MH): 279.0691; found, 279.0691.

### Example 49

(4R,6R,7S)-2-[(E)-2-cyclopropylivinyl-4,6,7-trihydroxy-4,5,6,7-tetrahydrobenzo[b]thiophen-4-carboxylic acid (Ic-6). The experimental procedure used was the same as for compound Ie-1 (example 33), but using lactone V-12 as starting material (28 mg, 0.10 mmol) in 0.9 mL of THF and 0.5 mL of LiOH (aq.). Purification by HPLC using a semipreparative columm Merck ZORBAX ODS (212×25 mm), eluting with (30:70) acetonitrile-water and at a flow of 7 mL Yield=29 mg (97%). Mp: 103-105° C. [α]_{D}²⁰−4.4 (c1.2, in CH₃OH). ¹H NMR (500 MHz, D₂O) δ 6.58 (s, 1H), 6.54 (d, 1H, J=16.0 Hz), 5.66 (dd, 1H, J=16.0 and 9.5 Hz), 4.62 (d, 1H, J=8.0 Hz), 4.00 (ddd, 1H, J=8.0, 4.0 and 12.0 Hz), 2.34 (dd, 1H, J=13.5 and 12.0 Hz), 2.18 (dd, 1H, J=13.5 and 4.0 Hz), 1.53-1.46 (m, 1H), 0.77 (m, 2H) and 0.45 (m, 2H) ppm. ¹³C NMR (125 MHz, D₂O) δ 180.7 (C), 147.3 (C), 142.6 (C), 139.8 (CH), 138.6 (C), 123.9 (CH), 123.0 (CH), 76.2 (C), 75.1 (CH), 73.0 (CH), 43.8 (CH₂), 16.7 (CH) and 9.8 (2×CH₂) ppm. IR (KBr): 3390 (O—H) and 1722 (C═O) cm⁻¹. MS (ESI) m/z (%) 319 (MNa⁺). HRMS calcd for C₁₄H₁₆O₃SNa (MNa⁺): 319.0611; found, 319.0618.

### Example 50

(4R, 6R, 7S)-4,7-di(tert-butyldimethylsilyloxy)-2-phenyl-4,5,6,7-tetrahydrobenzo[b]thiophen-4,6-carbolactone (V-13). A Shlenck tube was charged with iodide VII (130 mg, 0.23 mmol), Pd(PPh₃)₄ (26 mg, 0.02 mmol) and dry dioxane (2.5 mL). Anhydrous K₂CO₃ (0.63 mL, 0.69 mmol, 1.1 M) and phenylboronic acid (35 mg, 0.28 mmol) was then added and the resultant solution was deoxygenated and heated at 100° C. for 2 h. After cooling to room temperature, the reaction mixture was filtered through a plug of Celite and the precipitate was washed with hexane. The filtrate and the washings were concentrated and the obtained residue was purified by columm chromatography over silica gel, previously neutralized with triethylamine-hexanes (5:95), using diethyl ether-hexanes (5:95) as eluent. It was obtained 89 mg (75%) of thiophene V-13 as white foam. Mp: 58-61° C. [α]_{D}²⁰−47.8° (e1.1, in CHCl₃). ¹H NMR (300 MHz, CDCl₃) δ 7.58 (d, 2H, J=6.3 Hz), 7.40 (m, 2H), 7.32 (m, 1H), 7.26 (s, 1H), 4.93 (d, 1H, J=2.4 Hz), 4.72 (dd, 1H, J=2.4 and 4.5 Hz), 2.67 (d, 1H, J=8.4 Hz), 2.57 (dd, 1H, J=8.4 and 4.5 Hz), 1.04 (s, 9H), 0.97 (s, 9H), 0.32 (s, 3H), 0.28 (s, 3H), 0.25 (s, 3H) and 0.21 (s, 3H) ppm. ¹³C NMR (75 MHz, CDCl₃) δ 175.1 (C), 145.0 (C), 142.5 (C), 133.9 (2×C), 128.8 (2×CH), 127.9 (CH), 125.9 (2×CH), 118.9 (CH), 77.2 (CH), 75.5 (C), 66.3 (CH), 37.8 (CH₂), 25.7 (2×C(CH₃)₃), 18.2 (C(CH₃)₃), 18.0 (C(CH₃)₃), −3.0 (CH₃), −3.2 (CH₃) and −4.6 (2×CH₃) ppm. IR (KBr): 1803 (C═O)cm⁻¹. MS (CI) m/z (%) 517 (MH⁺). HRMS calcd for C₂₇H₄₁O₄SSi₂ (MH⁺): 517.2264; found, 517.2245.

### Example 51

(4R,6R,7S)-4,6,7-trihyclroxy-2-phenyl-4,5,6,7-tetrahydroben-zo[b]thiophen-4-carboxylic acid (Ic-7). To a stirred solution of the silyl ether V-13 (81 mg, 0.16 mmol) in dry THF (2.2 mL), under argon at 0° C., was added tetrabutylammonium fluoride (0.41 mL, 0.41 mmol, ca 1.0 M en THF). After stirring for 15 min at 0° C. the ice bath was removed and the reaction mixture was stirred for 1 h. The solvent was concentrated and the obtained residue was disolved in ethyl acetate and water. The mixture was acidified with dilute HCl until pH 1. The organic layer was separated and the aqueous phase was extracted with ethyl acetate (×2). The combined organic extracts were dried (anh. Na₂SO₄), filtered and concentrated under reduced pressure. The obtained residue was purified by flash chromatography over silica gel eluting with diethyl ether-hexanes (70:30) to yield (4R,6R,7S)-4,7-dihydroxy-2-phenyl-4,5,6,7-tetrahydrobenzo[b]thiophen-4,6-carbolactone (V-14, 40 mg, 89%) as an colourless oil. Carbolactone V-14 (40 mg, 0.14 mmol) was hydrolized as for compound Ic-1 using 0.55 mL of LiOH (aq.) and 0.7 mL of THF. Yield=38 mg (89%). White solid. Mp: 168-173° C. [(4°-20.2° (c1.0, in CH₂OH). NMR (400 MHz, CD₃OD) δ 7.62 (m, 2H), 7.42 (m, 2H), 7.32 (m, 1H), 7.17 (s, 1H), 4.66 (d, 1H, J=8.0 Hz), 4.13 (m, 1H), 2.50 (dd, 1H, J=13.6 and 11.6 Hz), and 2.27 (dd, 1H, J=13.6 and 3.2 Hz) ppm. ¹³C NMR (75 MHz, CDCl₃) δ 177.7 (C), 145.8 (C), 143.8 (C), 138.6 (C), 135.6 (C), 130.0 (2×CH), 128.8 (CH), 126.5 (2×CH), 122.0 (CH), 74.3 (C), 74.1 (CH), 71.7 (CH) and 42.6 (CH₂) ppm. IR (KBr): 3419 (O—H) and 1716 (C═O) cm'. MS (ESI) m/z (%) 329 (MNa⁺). HRMS calcd for C₁₅H₁₄O₅SNa (MNa): 329.0454; found, 329.0445.

### Example 52

(4R,6R,7S)-4,7-di(tert-butyldimethylsilyloxy)-2-(2-cyclopropyl)ethyl-4,5,6,7-tetrahydrobenzo[b]thiophen-4,6-carbolactone (V-15). A suspension of thiophene V-11 (78 mg, 0.15 mmol) and 10% palladium-on-carbon (16 mg) in methanol (1.5 mL) was stirred under hydrogen atmosphere at room temperature for 48 h. The mixture was filtered over Celite and the residue was washed with methanol. The filtrate and washings were evaporated to yield thiophene V-15 (75 mg, 96%) as a colourless oil. [α]_{D}²⁰−68.0° (c1.1, in CHCl₃). ¹H NMR (250 MHz, CDCl₃) δ 6.73 (s, 1H), 4.81 (d, 1H, J=3.3 Hz), 4.64 (dd, 1H, J=5.8 and 3.3 Hz), 2.86 (t, 2H, J=5.8 Hz), 2.59 (d, 1H, J=11.0 Hz), 2.48 (dd, 1H, J=11.0 and 6.0 Hz), 1.55 (m, 2H), 0.98 (s, 9H), 0.92 (s, 9H), 0.73 (m, 1H), 0.44 (m, 2H), 0.25 (s, 3H), 0.21 (s, 3H), 0.18 (s, 3H), 0.14 (s, 3H) and 0.07 (m, 2H) ppm. ¹³C NMR (63 MHz, CDCl₃) δ 175.5 (C), 146.7 (C), 141.2 (C), 131.8 (C), 120.0 (CH), 77.4 (CH), 75.6 (C), 66.3 (CH), 37.9 (CH₂), 36.7 (CH₂), 30.4 (CH₂), 25.7 (2×C(CH₃)₃), 18.2 (C(CH₃)₃), 18.1 (C(CH₃)₃), 10.6 (CH), 4.5 (2×CH₂), −3.1 (SiCH₃), −3.2 (SiCH₃) and −4.7 (2×SiCH₃) ppm. IR (film): 1801 (C═O) cm⁻¹. MS (CI) m/z (%) 509 (MH). HRMS calcd for C₂₆H₄₅O₄SSi₂ (MH⁺): 509.2577; found, 509.2566.

### Example 53

(4R,6R,7S)-2-(2-cyclopropyl)ethyl-4,7-dihydroxy-4,5,6,7-tetra-hydrobenzo[b]thiophen-4,6-carbolactone (V-16). The experimental procedure used was the same as for compound V-2 (example 32), but using silyl ether V-15 as starting material (38 mg, 0.075 mmol) in 1.1 mL of THF and 0.19 mL of TBAF. Purification by columm chromatography using diethyl ether-hexanes (70:30) as eluent. Yield=16.2 mg (77%). [α]_{D}²⁰−54.7” (c1.6, in MeOH ). ¹H NMR (250 MHz, CD,OD) δ 6.76 (s, 1H), 4.74 (dd, 1H, J=3.3 and 1.0 Hz), 4.70 (d, 1H, J=3.3 Hz), 2.84 (t, 2H, J=7.5 Hz), 2.43 (m, 2H), 1.49 (q, 2H, J=7.5 Hz), 0.70 (m, 1H), 0.39 (m, 2H) and 0.03 (m, 2H) ppm. ¹³C NMR (63 MHz, CD₃OD) δ 178.4 (C), 148.4 (C), 142.0 (C), 133.7 (C), 120.7 (CH), 79.2 (CH), 75.0 (C), 66.7 (CH), 38.5 (CH₂), 38.2 (CH₂), 31.3 (CH₂), 11.4 (CH) and 5.0 (2×CH₂) ppm.

### Example 54

(4R,6R,7S)-2-(2-cyclopropyl)ethyl-4,6,7-trihydroxy-4,5,6,7-tetra-hydrobenzo[b]thiophen-4-carboxylic acid (Ic-8). The experimental procedure used was the same as for compound Ic-1 (example 33), but using lactone V-16 as starting material (16 mg, 0.057 mmol) in 1 mL of THE and 0.17 mL of LiOH (aq.). Yield=10.4 mg (61%). [α]_{D}²⁰−24.9° (c1.0, in MeOH). ‘H NMR (250 MHz, D₂O) δ 6.41 (s, 1H), 4.54 (d, 1H, J=7.8 Hz), 3.91 (m, 1H), 2.74 (t, 2H, J=7.5 Hz), 2.23 (dd, 1H, J=13.5 and 12.3 Hz), 2.03 (dd, 1H, J=13.5 and 3.5 Hz), 1.41 (q, 2H, J=7.5 Hz), 0.63 (m, 1H), 0.28 (m, 2H) and −0.07 (m, 2H) ppm. ¹³C NMR (63 MHz, D₂O) δ 181.1 (C), 148.0 (C), 138.8 (C), 137.9 (C), 122.4 (CH), 74.6 (C), 72.6 (CH), 71.3 (CH), 41.6 (CH₂), 36.6 (CH₂), 30.3 (CH₂), 10.5 (CH) and 4.3 (2×CH₂) ppm. MS (EST) m/z (%) 271 (M−H). HRMS calcd for C₁₂H₁₅O₅S (M−H⁺): 271.0635; found, 271.0625.

### Example 55

(4R,6R,7R)-1-benzyl-4,7-di(tert-butyldimethylsilyloxy)-4,5,6,7-tetra-hydro-1H-indol-4,6-carbolactone (V-17). A solution of the obtained residue (100 mg) by ozonolysis of allyl derivative III-1 in acetic acid (1 mL), was treated with benzylamine (27 μL, 0.25 mmol) and was stirred at room temperature for 30 min. The reaction mixture was diluted with diethyl ether and sodium bicarbonate (sat.). The organic layer was separated and the aqueous phase was extracted with diethyl ether (2×). All the combined organic extracts were dried (anh. Na₂SO₄), filtered and concentrated under reduced pressure. The obtained residue was purified by columm chromatography eluting with diethyl ether-hexanes (10:90) to give indol V-17 (64 mg, 55%) as colourless oil. [α]_{D}²⁰−106.8° (c1.0, in CHCl₃). ¹H NMR (250 MHz, CDCl₃) δ 7.11 (m, 3H, 3×ArH), 6.80 (d, 2H, J=7.5 Hz, 2×ArH), 6.32 (d, 1 H, J=2.8 Hz, ArH), 6.04 (d, 1H, J=2.8 Hz, ArH), 4.92 (d, 1H, J=16.0 Hz, CHH), 4.84 (d, 1H, J=16.0 Hz, CHH), 4.57 (d, 1H, J=3.3 Hz, H-4), 4.48 (dd, 1H, J=6.0 and 3.3 Hz, H-5), 2.42 (d, 1H, J=10.8 Hz, H-6_{eq}), 2.31 (dd, 1H, J=10.8 and 6.0 Hz, H-6ₐₓ), 0.82 (s, 9H, C(CH₃)₃), 0.69 (s, 9H, C(CH₃)₃), 0.09 (s, 3H, SiCH₃), 0.00 (s, 3H, SiCH₃), −0.04 (s, 3H, SiCH₃) and −0.13 (s, 3H, SiCH₃) ppm. ¹³C NMR (63 MHz, CDCl₃) δ 176.1 (C), 137.2 (C), 128.8 (2×CH), 127.6 (CH), 126.5 (2×CH), 124.7 (C), 124.2 (C), 122.3 (CH), 103.6 (CH), 76.8 (CH), 74.2 (C), 64.4 (CH), 50.3 (NCH₂), 38.4 (CH₂), 25.7 (C(CH₃)₃), 25.6 (C(CH₃)₃), 18.2 (C(CH₃)₃), 17.9 (C(CH₃)₃), −2.9 (SiCH₃), −3.0 (SiCH₃), −4.1 (SiCH₃) and −4.6 (SiCH₃) ppm. IR (film): 1799 (C═O) cm⁻¹. MS (ESI) m/z (%) 514 (MH⁺). HRMS calcd for C₂₈H₄₄O₄Si₂N (WO: 514.2819; found, 514.2803.

### Example 57

(4R,6R,7R)-1-benzyl-4,7-dihydroxy-4,5,6,7-tetrahydro-1H-indol-4,6-carbolactone (V-18). The experimental procedure used was the same as for compound V-2 (example 32), but using silyl ether V-17 as starting material (80 mg, 0.16 mmol) in 1.5 mL of THF and 0.41 mL of TBAF. Purification by columm chromatography using ethyl acetate-hexanes (60:40) as eluent. Yield=40 mg (91%). [α]_{D}²⁰−91.6° (c1.0, in MeOH). ¹H NMR (250 MHz, CD₃OD) δ 7.29 (m, 3H), 7.13 (m, 2H), 6.70 (d, 1H, J=2.8 Hz), 6.18 (d, 1H, J=2.8 Hz), 5.20 (d, 1H, J=15.8 Hz), 5.13 (d, 1H, J=15.8 Hz), 4.69 (m, 1H), 4.63 (br s, 1H), 4.54 (d, 1H, J=3.3 Hz) and 2.46 (m, 2H) ppm. ¹³C NMR (63 MHz, CD₃OD) δ 179.5 (C), 139.2 (C), 129.8 (2×CH), 128.7 (CH), 128.3 (2×CH), 126.2 (C), 124.4 (C), 123.9 (CH), 103.59 (CH), 79.3 (CH), 73.8 (C), 64.4 (CH), 51.7 (CH₂) and 39.3 (CH₂) ppm. IR (film): 3395 (O—H) and 1784 (C═O) cm⁻¹. MS (ESI) m/z (%) 286 (MH⁺). HRMS calcd for C₁₆H₁₆O₄N (MH⁺): 286.1069; found, 286.1074.

### Example 58

(4R,6R,7S)-4,6,7-trihydroxy-2-isopropyl-4,5,6,7-tetrahydro-benzo[b]thiophen-4-carboxylic acid (Ic-8). A suspension of thiophene Ic-5 (20 mg, 0.074 mmol) and 10% palladium-on-carbon (4 mg) in methanol (0.7 mL) was stirred under hydrogen atmosphere at room temperature for 48 h. The mixture was filtered over Celite and the residue was washed with methanol. The filtrate and washings were concentrated under reduced pressure. The obtainded residue was purified by HPLC using a semipreparative columm Merck ZORBAX ODS (212×25 mm), eluting with (20:80) acetonitrile-water and at a flow of 7 mL min⁻¹ to afford thiophene Ic-8 (15 mg, 74%) as a white solid. [α]_{D}^{°}−6.4° (c1.0, in MeOH). Mp: 125-127° C. ¹H NMR (250 MHz, D₂O) δ 6.51 (s, 1H), 4.57 (d, 1H, J=8.3 Hz), 3.94 (m, 1H), 3.01 (quint, 1H, J=6.8 Hz), 2.30 (dd, 1H, J=11.8 and 13.5 Hz), 2.14 (dd, 1H, J=3.3 and 13.5 Hz) and 1.16 (d, 6H, J=6.8 Hz) ppm. ¹³C NMR (63 MHz, D₂O) δ 177.9 (C), 156.2 (C), 139.6 (C), 134.8 (C), 119.5 (CH), 73.5 (C), 72.4 (CH), 70.3 (CH), 41.2 (CH₂), 30.1 (CH) and 24.2 (CH₃) and 24.1 (CH₃) ppm. IR (KBr): 3398 (O—H) and 1724 (C═O) cm⁻¹. MS (ESI) m/z (%) 271 (M−H). HRMS calcd for C₁₂H₁₅O₅S (M−H⁺): 271.0635; found, 271.0639.

### Example 59

(4R,6R,7S)-2-ethyl-4,6,7-trihydroxy-4,5,6,7-tetrahydrobenzo[b]thiophen-4-carboxylic acid (Ic-9). The experimental procedure used was the same as for compound Ic-8 using vinyl thiophene Ic-3 (18 mg, 0.070 mmol) and 10% palladium-on-carbon (4 mg) in metanol (0.7 mL). Yield=13.9 mg (77%). [α]_{D}²⁰−34.6° (c1.0, in MeOH). ¹H NMR (250 MHz, D₂O) δ 6.40 (br s, 1H), 4.55 (d, 1H, J 7.3 Hz), 3.91 (m, 1H), 2.66 (q, 2H, J7.3 Hz), 2.20 (m, 1H), 2.04 (m, 1H) and 1.12 (t, 3H, J7.3 Hz) ppm. ¹³C NMR (63 MHz, D₂O) δ 180.8 (C), 149.6 (C), 138.4 (C), 137.6 (C), 121.2 (CH), 74.3 (C), 72.3 (CH), 71.0 (CH), 41.3 (CH₂), 23.4 (CH₂) and 15.4 (CH₃) ppm.

### Example 60

Ethyl (1R,4S,5R)-2-allyl-3-(benzo[b]thiophen-2-yl)methoxy-1,4,5-trihydroxycyclohex-2-en-1-carboxylate (Ib-9). A solution of lactone IV-10 (16 mg, 0.04 mmol) and sodium ethoxide (4 mg, 0.06 mmol) in ethanol (0.4 mL) was stirred at room temperature for 1 hora. Ethyl acetate and water was then added. The organic layer was separated and the aqueous phase was extracted with ethyl acetate (×2). All the combined organic extracts were dried (anh. MgSO₄), filtered and concentrated under reduced pressure. The obtained residue was purified by flash chromatography over silica gel eluting with ethyl acetate-hexanes (50:50). 9 mg (50%) of esthcr Ib-9 were obtained as white solid. ¹H NMR (400 MHz, CDCl₃) δ 7.78 (dd, 1H, J=6.8 and 1.6 Hz), 7.70 (dd, 1H, J=7.6 and 1.6 Hz), 7.32 (m, 2H), 7.23 (s, 1H), 5.72-5.62 (m, 1H), 5.27 (dd, 1H, J=12.4 and 0.8 Hz), 5.11 (d, 1H, J=12.4 Hz), 4.98 (dd, 1H, J=17.2 and 2.0 Hz), 4.89 (dd, 1H, J=10.0 and 1.6 Hz), 4.28 (d, 1H, J=7.2 Hz), 4.17-4.00 (m, 3H), 3.79 (br s, 1H), 2.91 (dd, 1H, J=14.8 and 6.8 Hz), 2.65 (ddd, 1H, J=14.8, 6.4 and 1.2 Hz), 2.08 (dd, 1H, J=13.2 and 11.6 Hz), 2.00 (dd, 1H, J=13.2 and 4.0 Hz) and 1.12 (t, 3H, J=6.8 Hz) ppm. ¹³C NMR (100 MHz, CDCl₃) δ 175.1 (C), 150.6 (C), 140.3 (C), 140.2 (C), 139.2 (C), 136.3 (CH), 124.5 (CH), 124.3 (CH), 123.7 (CH), 123.5 (CH), 122.3 (CH), 120.5 (C), 115.5 (CH₂), 75.4 (C), 72.0 (CH), 70.4 (CH), 67.0 (CH₂), 62.4 (CH₂), 39.6 (CH₂), 31.2 (CH₂) and 13.8 (CH₃) ppm.

(2-Bromomethyl)thiophene (15a). A solution of thien-2-ylmethanol (14a) (1 mL, 10.55 mmol), phosphorus tribromide (1.5 mL, 15.83 mmol) and three drops of dry pyridine in dry dichloromethane (40 mL), under argon and 0° C., was stirred for 1 h. During this period, the reaction mixture was allowed to reach room temperature. Water was then added and the organic phase was separated. The aqueous layer was extracted with dichloromethne (×2). All the combined organic extracts were washed with saturated sodium bicarbonate, dried (anh. Na₂SO₄), filtered and concentrated under reduced pressure to afford bromide 15a (1.5 g, 80%) as light yellow oil. ¹H NMR (250 MHz, CDCl₃) δ 7.73 (dd, 1H, J=5.0 and 1.0 Hz, H-5), 7.52 (d, 1H, J=3.0 Hz, H-3), 7.35 (dd, 1H, J=5.0 and 3.0 Hz, H-4) and 5.16 (s, 2H, CH₂Br) ppm. ¹³C NMR (63 MHz, CDCl₃) δ 141.9 (C), 131.8 (CH), 128.4 (CH), 128.2(CH) and 26.7 (CH₂) ppm.

(5-Bromomethyl)benzo[b]thiophene (15b). To a stirred solution of benzo[b]thiophen-5-ylmethanol (14b) (100 mg, 0.61 mmol) in dry dichloromethane (15 mL) and under argon, PPh₃ (290 mg, 1.09 mmol) and then CBr₄ (240 mg, 0.73 mmol) were added. After stirring for 1 h, diethyl ether was added and the resulting precipitate was filtered and washed with diethyl ether. The solvents were removed under reduced pressure and the crude residue was purified by flash chromatography eluting with (10:90) diethyl ether/hexanes to afford bromide 15b (129 mg, 93%) as a white amorphous solid. ¹H NMR (250 MHz, CDCl₃) δ 7.86 (m, 2H, 2×ArH), 7.49 (d, 1H, J=5.5 Hz, ArH), 7.39 (d, 1H, J=8.7 Hz, ArH), 7.33 (d, 1H, J=5.5 Hz, ArH) and 4.66 (s, 2H, CH₂O) ppm. ¹³ C NMR (63 MHz, CDCl₃) δ 139.7 (C), 139.7 (C), 133.8 (C), 127.4 (CH), 125.2 (CH), 123.9 (CH), 123.7 (CH), 122.8 (CH) and 34.1 (CH₂) ppm. MS (CI) m/z (%) 227 and 229 (MH⁺). HRMS calcd for C₉H₃S⁷⁹Br (MH⁺): 226.9530; found, 226.9532.

### Example 61

(1R,4S,5R)-1,4-ditert-butyldimethylsilyloxy-3-(thien-2-yl)methoxycyclohex-2-en-1,5-carbolactone (1a) and (1R,4S,5R)-1,4-ditert-butyldimethylsilyloxy-3-(thien-2-yl)methoxy-2-(thien-2-yl)methylcyclohex-2-en-1,5-carbolactone (2a). The experimental procedure used was the same as for compounds IV-1 and IV-2 in example 17, using the following: First, 300 mg of (1R,4S,5R)-1,4-di(tert-butyldimethylsilyloxy)-3-oxocyclohexan-1,5-carbolactone (0.75 mmol) in 1.5 mL of DMF and 3.0 mL of KHMDS (1.5 mmol) was used for the enolate generation. In the alkylation step, 265 mg of 2-(bromomethyl)thiophene (1.50 mmol) in 1.3 mL of DMF was used. Yield=67 mg (18%) of la and 153 mg (35%) of 2a.

Data for 1a: White solid. Mp: 65-67° C. [α]_{D}²⁰=−133° (c1.2, in CHCl₃). ¹H NMR (250 MHz, CDCl₃) δ 7.31 (dd, 1H, J=5.0 and 1.2 Hz, ArH), 7.03 (d, 1H, J=2.8 Hz, ArH), 6.98 (dd, 1H, J=5.0 and 3.8 Hz, ArH), 5.01 (s, 1H, H-2), 4.90 (d, 1H, J=11.5 Hz, CHHAr), 4.84 (d, 1H, J=11.5 Hz, CHHAr), 4.47 (dd, 1H, J=5.2 and 3.5 Hz, H-5), 4.14 (d, 1H, J=3.5 Hz, H-4), 2.40 (d, 1H, J=10.5 Hz, H-6ₐₓ), 2.33 (dd, 1H, J=10.5 and 5.2 Hz, H-6,), 0.93 (s, 9H, C(CH₃)₃), 0.87 (s, 9H, C(CH₃)₃), 0.19 (s, 3H, SiCH₃), 0.15 (s, 3H, SiCH₃), 0.08 (s, 3H, SiCH₃) and 0.05 (s, 3H, SiCH₃) ppm. ¹³C NMR (63 MHz, CDCl₃) δ 176.1 (C), 153.2 (C), 137.7 (C), 127.1 (CH), 126.5 (CH), 126.3 (CH), 104.8 (CH), 75.2 (CH), 73.6 (C), 67.3 (CH), 64.3 (CH₂), 37.9 (CH₂), 25.6 (C(CH₃)₃), 25.5 (C(CH₃)₃), 18.0 (2×C(CH₃)₃), −3.1 (2×CH3), −4.6 (CH₃) and −5.3 (CH₃) ppm. IR (KBr) 1801 (C═O) cm⁻¹. MS (Cl) m/z (%) 497 (MH⁺). HRMS calcd for C₂₄H₄₁O₅SSi₂ (MH⁻): 497.2213; found, 497.2214.

Data for 2a: Yellow oil. [α]_{D}²⁰=−127° (c1.0, in CHCl₃). ¹H NMR (300 MHz, CDCl₃) δ 7.29 (d, 1H, J=4.2 Hz, ArH), 7.06 (d, 1H, J=4.5 Hz, ArH), 6.92 (m, 3H, 3×ArH), 6.77 (s, 1H, ArH), 4.96 (s, 2H, CH₂O), 4.54 (m, 1H, H-5), 4.42 (br s, 1H, H-4), 3.79 (d, 1H, J=15.0 Hz, CHHAr), 3.69 (d, 1H, J=15.0 Hz, CHHAr), 2.53 (d, 1H, J=10.8 Hz, H-6ₐₓ), 2.41 (dd, 1H, J=10.8 and 5.7 Hz, H-6_{eq}), 0.95 (s, 9H, C(CH₃)₃), 0.80 (s, 9H, C(CH₃)₃), 0.20 (s, 3H, CH₃), 0.18 (s, 3H, CH₃), 0.17 (s, 3H, CH₃) and 0.06 (s, 3H, CH₃) ppm. ¹³C NMR (75 MHz, CDCl₃) δ 175.3 (C), 148.2 (C), 142.8 (C), 138.8 (C), 129.7 (C), 126.9 (CH), 126.7 (CH), 126.4 (CH), 126.3 (CH), 124.5 (CH), 122.8 (CH), 74.6 (CH+C), 67.5 (CH₂), 67.2 (CH), 37.5 (CH₂), 25.7 (C(CH₃)₃), 25.5 (C(CH₃)₃), 24.7 (CH₂), 18.1 (C(CH₃)₃), 18.0 (C(CH₃)₃), −3.3 (CH₃), −3.5 (CH₃) and −4.5 (2×CH₃) ppm. IR (film) 1799 (C═O) cm⁻¹. MS (CI) m/z (%) 593 (MH⁺). HRMS calcd for C₂₉H₄₅O₃S₂Si₂ (MH⁻): 593.2247; found, 593.2248.

### Example 62

(1R,4S,5R)-1,4-ditert-butyldimethylsilyloxy-3-(benzo[b]thiophen-5-yl)methoxycyclohex-2-en-1,5-carholactone (1b) and (1R,4S,5R)-1,4-ditert-butyldimethylsilyloxy-3-(benzo[b]thiophen-5-yl)methoxy-2-(benzo[b]thiophen-5-yl)methylcyclohex-2-en-1,5-carbolactone (2 h). The reaction was carried out as for compounds 1a and 2a by using 500 mg of (1R,4S,5R)-1,4-di(tert-butyldimethylsilyloxy)-3-oxocyclohexan-1,5-carbolactone and 570 mg of (5-bromomethyl)benzo[b]thiophene as starting materials. Yield: 173 mg (25%) of O-alkyl derivative 1b and 237 mg (27%) of dialkyl derivative 2b.

Data for 1b: Colourless oil. [α]_{D}²⁰=−108+ (c1.2, in CHCl₃). ¹H NMR (250 MHz, CDCl₃) δ 7.79 (d, 1H, J=8.2 Hz, ArH), 7.72 (s, 1H, ArH), 7.39 (d, 1H, J=5.2 Hz, ArH), 7.24 (m, 2H, 2×ArH), 4.97 (s, 1H, H-2), 4.82 (d, 1H, J=11.2 Hz, CHHO), 4.74 (d, 1H, J=11.2 Hz, CHHO), 4.43 (dd, 1H, J=5.0 and 3.5 Hz, H-5), 4.13 (d, 1H, J=3.5 Hz, H-4), 2.35 (d, 1H, J=10.5 Hz, H-6ₐₓ), 2.28 (m, 1H, H-6_{eq}), 0.87 (s, 9H, C(CH₃)₃), 0.82 (s, 9H, C(CH₃)₃), 0.09 (s, 3H, SiCH₃), 0.04 (s, 3H, SiCH₃), 0.02 (s, 3H, SiCH₃) and −0.01 (s, 3H, SiCH₃) ppm. ¹³C NMR (63 MHz, CDCl₃) δ 176.1 (C), 153.4 (C), 139.6 (C), 139.3 (C), 131.9 (C), 127.0 (CH), 124.0 (CH), 123.7 (CH), 122.8 (CH), 122.4 (CH), 104.9 (CH), 75.2 (C), 73.7 (C), 69.6 (CH₂), 67.4 (CH), 38.0 (CH₂), 25.6 (C(CH₃)₃), 25.5 (C(CH₃)₃), 18.0 (C(CH₃)₃), 17.9 (C(CH₃)₃), −3.2 (2×SiCH3), −4.6 (SiCH₃) and −5.2 (SiCH₃) ppm. IR (film): 1801 (C═O) cm⁻¹. MS (Cl) m/z (%) 547 HRMS calcd for C₂₈H₄₃O₅SSi₂ (MH): 547.2370; found, 547.2375.

Data for 2b: Colourless oil. [α]_{D}²⁰=−87° (c1.7, in CHCl₃). ¹H NMR (250 MHz, CDCl₃) δ 7.77 (d, 1H, J =8.5 Hz, ArH), 7.69 (d, 1H, J=8.5 Hz, ArH), 7.57 (s, 1H, ArH), 7.55 (s, 1H, ArH), 7.43 (d, 1H, J=5.5 Hz, ArH), 7.36 (d, 1H, J=5.5 Hz, ArH), 7.23-7.12 (m, 4H, 4×ArH), 4.89 (d, 1H, J=11.2 Hz, OCHH), 4.77 (d, 1H, J=11.2 Hz, OCHH), 4.53 (dd, 1H, J=5.5 and 3.5 Hz, H-5), 4.48 (d, 1H, J=3.5 Hz, H-4), 3.81 (d, 1H, J=15.0 Hz, CHHAr), 3.66 (d, 1H, J=15.0 Hz, CHHAr), 2.56 (d, 1H, J=10.8 Hz, H-6ₐₓ), 2.44 (dd, 1H, J=10.8 Hz and 5.5 Hz, H-6_{eq}), 0.95 (s, 9H, C(CH₃)₃), 0.73 (s, 9H, C(CH₃)₃), 0.17 (s, 3H, SiCH₃), 0.16 (s, 3H, SiCH₃), 0.13 (s, 3H, SiCH₃) and 0.03 (s, 3H, SiCH₃) ppm. ¹³C NMR (63 MHz, CDCl₃) δ 175.6 (C), 148.7 (C), 139.6 (C), 139.5 (C), 136.9 (C), 136.1 (C), 132.9 (C), 129.2 (C), 128.5 (C), 126.7 (CH), 125.9 (CH), 125.3 (CH), 123.8 (CH), 123.6 (CH), 123.5 (CH), 123.1 (CH), 122.3 (CH), 122.2 (CH), 121.7 (CH), 74.9 (C), 72.6 (CH₂), 67.1 (CH), 37.6 (CH₂), 29.9 (CH₂), 25.6 (C(CH₃)₃), 25.4 (C(CH₃)₃), 17.9 (2×C(CH₃)₃), −3.4 (SiCH₃), −3.6 (SiCH₃) and −4.5 (2×SiCH₃) ppm. IR (film) 1799 (C═O) cm⁻¹. MS (CI) m/z (%) 693 (MH⁺). HRMS calcd for C₃₇H₄₉O₅S₂Si₂ (MH⁻): 693.2560; found, 693.2464.

### Example 63

(1R,4S,5R)-1,4-dihydroxy-3-(thien-3-yl)methoxycyclohex-2-en-1,5-carbolactone (3a). The experimental procedure used was the same as for compound IV-3 of example 18, but using silyl ether la as starting material (65 mg, 0.13 mmol) in 1.9 mL of THF and 0.29 mL de tetrabutylammonium fluoride (0.29 mmol). Yield=30 mg (86%). White foam. [α]_{D}²⁰=−189° (c1.5, in MeOH ). ¹H NMR (300 MHz, CD₃OD) δ 7.40 (dd, 1H, J=5.1 and 0.9 Hz, ArH), 7.12 (d, 1H, J=2.7 Hz, ArH), 6.99 (dd, 1H, J=5.1 and 3.6 Hz, ArH), 5.15 (s, 1H, H-2), 4.99 (d, 1H, J=12.3 Hz, CHHAr), 4.93 (d, 1H, J=12.3 Hz, CHHAr), 4.61 (m, 1H, H-5), 4.08 (d, 1H, J=3.3 Hz, H-4) and 2.32 (m, 2H, H-6) ppm. ¹³C NMR (75 MHz, CD₃OD) δ 179.2 (C), 155.3 (C), 146.0 (C), 139.6 (CH), 128.6 (CH), 127.7 (CH), 127.5 (CH), 127.5 (CH), 105.1 (CH), 76.9 (CH), 72.9 (C), 67.6 (CH₂), 65.5 (CH) and 38.3 (CH₂) ppm. IR (KBr) 3463 (O—H) and 1774 (C═O) cm⁻¹. MS (CI) m/z (%) 269 (MH⁺). HRMS calcd for C₁₂H₁₂O₅S (MH): 269.0484; found, 269.0480.

### Example 64

Sodium (1R,4S,5R)-1,4,5-trihydroxy-3-(thien-3-yl)methoxycyclohex-2-en-1-carboxylate (Ib-6). The experimental procedure used was the same as for compound Ib-1 in example 19, but using carbolactone 3a as starting material (29 mg, 0.11 mmol) in 1.0 mL of THF and 215 μL of NaOH (aq.). Yield=33 mg (97%). Beige solid. Mp: 125-128° C. [α]_{D}²⁰=−31° (c1.2, in H₂O). ¹H NMR (300 MHz, D₂O) δ 7.48 (br d, 1H, J=5.1 Hz, ArH), 7.19 (d, 1H, J=3.0 Hz, ArH), 7.07 (dd, 1H, J=5.1 and 3.6 Hz, ArH), 5.06 (d, 1H, J=12.0 Hz, CHHAr), 4.99 (d, 1H, J=12.0 Hz, CHHAr), 4.97 (s, 1H, H-2), 4.05 (d, 1H, J=7.5 Hz, H-4), 3.96 (m, 1H, H-5), 2.08 (dd, 1H, J=13.5 and 10.2 Hz, H-6ₐₓ) and 2.00 (dd, 1H, J=13.5 and 4.5 Hz, H-6_{eq}) ppm. ¹³C NMR (75 MHz, D₂O) δ 184.5 (C), 158.3 (C), 141.4 (C), 131.1 (CH), 130.3 (CH), 130.2 (CH), 103.7 (CH), 76.9 (C), 75.0 (CH), 72.6 (CH), 67.3 (CH₂) and 41.9 (CH₂) ppm. IR (KBr) 3419 (O—H), 1653 and 1601 (C═O) cm⁻¹.

### Example 65

(1R,4S,5R)-3-(benzo[b]thiophen-5-yl)methoxy-1,4-dihydroxycyclohex-2-en-1,5-carbolactone (3b). The experimental procedure used was the same as for compound 3a by using silyl ether 1b (173 mg, 0.32 mmol) as starting material and tetrabutylammonium fluoride (0.70 mL, 0.70 mmol) and THF (4.5 mL). Yield: 94 mg (92%). White solid. Mp: 177-179° C. [α]_{D}²⁰=−153° (c1.0, in MeOH ). ¹H NMR (250 MHz, CD₃OD) δ 7.81 (m, 2H, 2×ArH), 7.51 (d, 1H, J=5.5 Hz, ArH), 7.31 (m, 2H, 2×ArH), 5.08 (s, 1H, H-2), 4.84 (s, 2H, CH₂O), 4.57 (m, 1H, H-5), 4.08 (d, 1H, J=3.2 Hz, H-4) and 2.26 (m, 2H, H-6) ppm. ¹³C NMR (63 MHz, CD₃OD) δ 179.2 (C), 155.6 (C), 141.2 (C), 140.7 (C), 133.8 (C), 128.1 (CH), 125.1 (CH), 124.8 (CH), 123.8 (CH), 123.3 (CH), 104.9 (CH), 76.9 (CH), 72.9 (C), 70.9 (CH2), 67.7 (CH) and 38.3 (CH₂) ppm. IR (film): 3431 (O—H) and 1763 (C═O) cm'. MS (CI) m/z (%) 319 (MH). HRMS calcd for C₁₆H₁₅O₅S (MH⁺): 319.0640; found, 319.0640.

### Example 66

Sodium (1R,4S,5R)-1,4,5-trihydroxy-3[(benzo[b]thiophen-5-yl)methoxy]cyclohex-2-en-1-carboxylate (Ib-7). The experimental procedure used was the same as for compound Ib-6 using 20 mg (0.06 mmol) of lactone 3b, 125 μL of NaOH (aq.) and 0.6 mL of THF. Yield=21 mg (98%). White solid. Mp: 157-160° C. (dec.). [α]_{D}²⁰=−26° (c1.4, in H₂O). ¹H NMR (250 MHz, D₂O) δ 7.99 (d, 1H, J=8.5 Hz, ArH), 7.92 (s, 1H, ArH), 7.63 (d, 1H, J=5.2 Hz, ArH), 7.45 (s, 1H, ArH), 7.42 (m, 1H, ArH), 4.94 (m, 3H, CH₂O+H-2), 4.09 (d, 1H, J=7.2 Hz, H-4), 3.98 (m, 1H, H-5), 2.11 (dd, 1H, J=13.5 and 10.2 Hz, H-6ₐₓ) and 2.00 (dd, 1H, J=13.5 and 4.2 Hz, H-6_{eq}) ppm. ¹³C NMR (63 MHz, D₂O) δ 182.1 (C), 156.3 (C), 140.3 (C), 139.9 (C), 133.3 (C), 128.5 (CH), 125.0 (CH), 124.5 (CH), 123.8 (CH), 123.4 (CH), 101.1 (CH), 74.4 (C), 72.6 (CH), 70.4 (CH₂), 70.2 (CH) and 39.5 (CH₂) ppm. IR (KBr): 3419 (O—H), 1655 and 1597 (C═O) cm⁻¹. MS (ESI) m/z (%) 359 (MH⁺). HRMS calcd for C₁₆H₁₆O₆SNa (MH): 359.0560; found, 359.0560.

### Example 67

(1R,4S,5R)-1,4-dihydroxy-3-(thien-2-yl)methoxy-2-(thien-2-yl)methylcyclohex-2-en-1,5-carholactone (3c). The experimental procedure used was the same as for compound 3a using 145 mg (0.24 mmol) of lactone 2a, 540 μL of TBAF and 3.5 mL of THF. Yield=75 mg (86%). White solid. Mp: 95-97° C. [α]_{D}²⁰=−189° (c1.2, in MeOH ). ¹H NMR (300 MHz, CD₃OD) δ 7.36 (d, 1H, J=4.8 Hz, ArH), 7.07 (m, 1H, ArH), 7.02 (s, 1H, ArH), 6.96 (m, 1H, ArH), 6.81 (s, 2H, 2×ArH), 5.22 (d, 1H, J=12.3 Hz, OCHH), 5.11 (d, 1H, J=12.3 Hz, OCHH), 4.61 (m, 1H, H-5), 4.51 (d, 1H, J=2.4 Hz, H-4), 3.76 (d, 1H, J=14.4 Hz, CHHAr), 3.64 (d, 1H, J=14.4 Hz, CHHAr), 2.38 (d, 1H, J=11.1 Hz, H-6ₐₓ) and 2.32 (dd, 1H, J=11.1 and 6.0 Hz, H-6_{eq}) ppm. ¹³C NMR (75 MHz, CD₃OD) δ 178.8 (C), 148.5 (C), 144.1 (C), 141.0 (C), 127.9 (CH), 127.7 (CH), 127.3 (CH), 127.3 (CH), 126.2 (CH+C), 124.0 (CH), 76.8 (CH), 73.6 (C), 66.1 (CH), 65.9 (CH₂), 38.4 (CH₂) and 24.9 (CH₂) ppm. IR (KBr) 3498 (O—H), 3413 (O—H) and 1774 (C═O) cm⁻¹. MS (CI) m/z (%) 365 (MH⁺). HRMS calcd for C₁₇H₁₆O₅S₂ (MH): 365.0517; found, 365.0517.

### Example 68

Sodium (1R,4S,5R)-1,4-dihydroxy-3-(thien-2-yl)methoxy-2-(thien-2-yl)methylcyclohex-2-en-1-carboxylate (Ib-8). The experimental procedure used was the same as for compound Ib-6 using 25 mg (0.07 mmol) of lactone 3c, 140 μL of NaOH (aq.) and 0.6 mL of THF. Yield=25 mg (97%). Biege solid. Mp: 178-181° C. [α]_{D}²⁰=−89° (c1.2, in H₂O). ¹H NMR (300 MHz, D₂O) δ 7.46 (d, 1H, J=4.8 Hz, ArH), 7.19 (dt, 1H, J=5.1 and 1.2 Hz, ArH), 7.13 (br d, 1H, J=2.7 Hz, ArH), 7.05 (ddd, 1H, J=5.1, 3.6 and 0.6 Hz, ArH), 6.92 (dt, 1H, J=3.6 and 0.9 Hz, ArH), 6.85 (m, 1H, ArH), 5.18 (d, 1H, J=12.0 Hz, CHHO), 4.92 (d, 1H, J=12.0 Hz, CHHO), 4.37 (d, 1H, J=6.6 Hz, H-4), 3.93 (m, 1H, H-5), 3.68 (d, 1H, J=15.6 Hz, CHHAr), 3.28 (d, 1H, J=15.6 Hz, CHHAr), 2.16 (dd, 1H, J=13.8 and 11.4 Hz, H-6ₐₓ) and 2.03 (dd, 1H, J=13.8 and 3.3 Hz, H-6_{aq}) ppm. ¹³C NMR (75 MHz, D₂O) δ 182.9 (C), 153.8 (C), 146.8 (C), 142.0 (C), 131.2 (CH), 130.3 (CH), 130.2 (CH), 129.8 (CH), 128.4 (CH), 126.8 (CH), 126.7 (C), 79.8 (C), 73.0 (CH), 72.9 (CH), 68.4 (CH₂), 41.7 (CH₂) and 29.8 (CH₂) ppm. IR (KBr) 3390 (O—H) and 1597 (C═O) cm⁻¹. EM (ESI) m/z (%) 381 (M⁻). HRMS calcd for C₁₇H₁₇O₆S₂(M⁻): 381.0461; found, 381.0461.

### Example 69

(1R,4S,5R)-3-[(benzo[b]tiophen-5-yl)methoxyl-2-[(benzo[b]thiophen-5-yl)methyl]-1,4-dihydroxycyclohex-2-en-1,5-carbolactone (3d). The experimental procedure used was the same as for compound 3a using 97 mg of ether 2b (0.14 mmol), 280 μL of TBAF and 2.0 mL of THE. Yield=56 mg (86%). White solid. Mp: 123-125° C. [α]_{D}^{°}=−163° (c1.5, in acetone). ¹H NMR (250 MHz, CD₃OD) δ 7.74 (d, 1H, J=8.2 Hz, ArH), 7.61 (m, 3H, 3×ArH), 7.49 (d, 1H, J=5.5 Hz, ArH), 7.39 (d, 1H, J=5.5 Hz, ArH), 7.19 (m, 3H, 3×ArH), 7.10 (d, 1H, J=5.5 Hz, ArH), 5.13 (d, 1H, J=11.5 Hz, OCHH), 4.92 (d, 1H, J=11.5 Hz, OCHH), 4.57 (m, 1H, H-5), 4.51 (d, 1H, J=3.2 Hz, H-4), 3.73 (d, 1H, J=14.2 Hz, CHHAr), 3.56 (d, 1H, J=14.2 Hz, CHHAr) and 2.30 (m, 2H, H-6) ppm. ¹³C NMR (63 MHz, acetone-d6) δ 177.8 (C), 149.6 (C), 141.4 (C), 140.6 (C), 138.7 (C), 138.4 (C), 135.8 (C), 128.7 (CH), 127.7 (CH), 127.5 (CH), 125.8 (CH), 125.4 (CH), 125.3 (C), 125.3 (CH), 125.2 (CH), 125.1 (C), 124.2 (CH), 123.8 (CH), 123.1 (CH), 76.6 (CH), 74.2 (C), 71.4 (CH₂), 66.8 (CH), 38.9 (CH₂) and 30.9 (CH₂) ppm. IR (KBr) 3452 (O—H), 3363 (O—H) and 1770 (C═O) cm⁻¹. MS (CI) m/z (%) 30 465 (MH⁺). HRMS calcd for C₂₅H₂₁O₅S₂ (MH⁺): 465.0830; found, 465.0831.

### Example 70

Sodium (1R,4S,5R)-3[(benzo[b]thiophen-5-yl)methoxy]-2-[(benzo[b]thiophen-5-yl)methyl]-1,4-dihydroxycyclohex-2-en-1-carboxylate (Ib-9). The experimental procedure used was the same as for compound 3a using 25 mg (0.05 mmol) of lactone 3d, 0.1 mL of NaOH (aq.) and 0.4 mL of THF. Yield=25 mg (99%). White solid. Mp: 197-200° C. (dec.). [α]_{D}²⁰=−67° (c1.3, in 50% aqueous MeOH). ¹H NMR (250 MHz, 50% CD₃OD/D₂O) δ 7.66 (m, 3H, 3×ArH), 7.47 (m, 2H, 2×ArH), 7.26 (d, 1H, J=8.2 Hz, ArH), 7.14 (m, 2H, 2×ArH), 7.05 (d, 1H, J=5.5 Hz, ArH), 6.98 (d, 1H, J=8.2 Hz, ArH), 4.84 (d, 1H, J=10.5 Hz, OCHH), 4.58 (d, 1H, J=10.5 Hz, OCHH), 4.41 (d, 1H, J=5.0 Hz, H-4), 3.98 (m, 1H, H-5), 3.57 (d, 1H, J=15.7 Hz, CHHAr), 3.28 (d, 1H, J=15.7 Hz, CHHAr) and 2.20 (m, 2H, H-6) ppm. ¹³C NMR (63 MHz, 50% CD₃OD/D₂O) δ 181.9 (C), 153.1 (C), 141.9 (C), 141.6 (C), 141.0 (C), 139.7 (C), 138.8 (C), 135.3 (C), 128.9 (CH), 128.2 (CH), 127.6 (CH), 126.4 (CH), 125.8 (CH), 125.7 (C), 125.3 (CH), 125.0 (CH), 124.3 (CH), 123.9 (CH), 123.5 (CH), 78.5 (C), 72.2 (CH), 72.0 (CH₂), 70.9 (CH), 38.8 (CH₂) and 33.8 (CH₂) ppm. IR (KBr) 3410 (O—H) and 1595 (C═O) cm⁻¹. MS (ESI) m/z (%) 481 (M⁻). HRMS calcd for C₂₅H₂₁O₆S₂Na (M): 481.0774; found, 481.0776.

### Example 71

Methyl (1R,4S,5R)-3-(benzo[b]thiophen-5-yl)methoxy-2-(benzo[b]thiophen-5-yl)methyl-1,4,5-trihydroxycyclohex-2-enecarboxylate (Ib-15). A solution of the corresponding carbolactone 3d (57 mg, 0.12 mmol) in dry methanol (0.7 mL) and acetonitrile (0.7 mL) was treated with sodium methoxide (7 mg, 0.13 mmol). The resultant mixture was stirred at room temperature for 2 hour and then it was diluted with ethyl acetate and water. The organic layer was separated and the aqueous phase was extracted with ethyl acetate (×3). All the combined organic extracts were dried (anh. Na₂SO₄), filtered and concentrated under reduced pressure. The obtained residue was purified by flash chromatography eluting with (1:1) ethyl acetate/hexanes to afford methyl ester Ib-15 (24 mg, 40%) as white solid. Mp: 148-152° C. [α]_{D}²⁰+9° (c1.2, in MeOH). ¹H NMR (300 MHz, CD₃OD) δ 7.72 (d, 1H, J=8.4 Hz, ArH), 7.60 (m, 2H, 2×ArH), 7.52 (br s, 1H, ArH), 7.48 (d, 1H, J=5.4 Hz, ArH), 7.42 (d, 1H, J=5.4 Hz, ArH), 7.23 (dd, 1H, J=8.1 and 1.5 Hz, ArH), 7.17 (dd, 1 H, J=5.7 and 0.6 Hz, ArH), 7.11 (m, 2H, 2×ArH), 5.26 (d, 1H, J=11.1 Hz, OCHH), 4.87 (d, 1H, J=11.1 Hz, OCHH), 4.38 (d, 1H, J=7.5 Hz, H-4), 4.03 (m, 1H, H-5), 3.55 (d, 1H, J=15.3 Hz, CHHAr), 3.38 (d, 1H, J=15.3 Hz, CHHAr), 3.14 (s, 3H, OCH₃), 2.18 (dd, 1H, J=13.2 and 12.0 Hz, H-6ₐₓ) and 1.99 (dd, 1H, J=13.2 and 3.9 Hz, H-6_{eq}) ppm. ¹³C NMR (75 MHz, CD₃OD) δ 176.6 (C), 154.0 (C), 141.2 (2xC), 140.6 (C), 138.5 (C), 137.5 (C), 135.1 (C), 127.9 (CH), 127.2 (CH), 127.0 (CH), 125.9 (CH), 124.9 (CH), 124.7 (2×CH), 124.4 (CH), 123.2 (CH), 122.5 (CH), 120.8 (C), 77.0 (C), 72.9 (CH), 72.2 (CH₂), 71.3 (CH), 52.7 (OCH;), 41.5 (CH₂) and 33.1 (CH₂) ppm. IR (KBr) 3410 (O—H) and 1734 (C═O) cm⁻¹. MS (ESI) m/z (%) 519 (MNa⁺). HRMS calcd for C₂₆H₂₄O₆S₂Na (MNa⁺): 519.0907; found, 519.0901.

### Example 72

Methyl (1R,4S,5R)-1,4,5-trihydroxy-3-(thien-3-il)methoxy-2-(thien-3-yl)methyl cyclohex-2-en-1-carboxylate (Ib-16). The experimental procedure used was the same as for compound Ib-15 using 40 mg of lactone 3c, 6 mg of NaOMe and 1.2 mL of methanol. Yield: 22 mg (51%). Beige solid. Mp: 55-57° C. [α]_{D}²⁰=+7° (c1.1, in MeOH). ¹H NMR (300 MHz, CD₃OD) δ 7.34 (dd, 1H, J=5.1 and 1.2 Hz, ArH), 7.10 (dd, 1H, J=5.1 and 1.2 Hz, ArH), 7.04 (m, 1H, ArH), 6.95 (dd, 1H, J=5.1 and 3.3 Hz, ArH), 6.80 (dd, 1H, J=5.1 and 3.3 Hz, ArH), 6.67 (m, 1H, ArH), 5.37 (d, 1H, J=11.4 Hz, CHHO), 5.01 (d, 1H, J=11.4 Hz, CHHO), 4.25 (d, 1H, J=7.8 Hz, H-4), 3.93 (m, 1H, H-5), 3.61 (d, 1H, J=15.3 Hz, CHHAr), 3.43 (d, 1H, J=15.3 Hz, CHHAr), 3.33 (s, 3H, OCH₃), 2.14 (dd, 1H, J=12.9 and 12.0 Hz, H-6ₐₓ) and 1.95 (dd, 1H, J=12.9 and 3.9 Hz, H-6_{ec}) ppm. ¹³C NMR (75 MHz, CD₃OD) δ 176.2 (C), 153.7 (C), 144.1 (C), 141.1 (C), 128.1 (CH), 127.4 (CH), 127.2 (CH), 126.9 (CH), 126.4 (CH), 124.3 (CH), 120.9 (C), 76.3 (C), 72.7 (CH), 71.1 (CH), 66.5 (CH₂), 52.8 (CH₃), 41.3 (CH₂) and 27.2 (CH₂) ppm. IR (KBr) 3435 (O—H) and 1734 (C═O) cm⁻¹.

### Example 73

(1R,4S,5R)-1,4-di(tert-butyldimethylsilyloxy)-3-(benzo[b]thiophen-2-yl)methoxy-2-(thien-2-yl)methylcyclohex-2-en-1,5-carbolactone (20. A solution of (1R,4S,5R)-1,4-di(tert-butyldimethylsityloxy)-3-oxocyclohexan-1,5-carbolactone (500 mg, 1.25 mmol) in dry THF (12.5 mL) under inert atmosphere, was treated with a solution of LHMDS (1.9 mL, 1.87 mmol, 1M in THF). The resultant mixture was stirred at room temperature for 1 h and it was then treated with a solution of 2-iodomethylthiophene (560 mg, 2.5 mmol) in dry THF (4 mL). After 30 min., the solvent was removed and the obtained residue was purified by flash chromatography eluting with diethyl ether/hexanes (5:95) to yield a mixture of C-alkylated products (206 mg, 33%). A solution of 50 mg (0.10 mmol) of the later mixture in DMF (0.5 mL) and toluene (0.5 mL), both dry, was treated with a solution of KHMDS (0.4 mL, 0.5 M in toluene) in dry DMF (0.2 mL). After 20 min, a solution of 2-(bromomethyl)benzo[b]thiophene (46 mg, 0.20 mmol) in 0.5 mL of DMF and 0.3 mL of toluene, both dry. After 40 min., water and diethyl ether were added, the organic layer was separated and the aqueous layer was extracted with diethyl ether (×3). All the combined organic extracts were dried (anh. Na₂SO₄), filtered and concentrated under reduced pressure. The obtained residue was purified by flash chromatography eluting with a gradient of diethyl ether/hexanes)[1°) (0-100%), 2°) (5-95%)] to afford lactone 2c (28 mg, 44%) as a yellow oil. [α]_{D}^{°}=−132° (c1.2, in CHCl₃). ¹H NMR (250 MHz, CD₃OD) δ 7.80 (m, 1H, ArH), 7.72 (m, 1H, ArH), 7.32 (m, 2H, 2×ArH), 7.07 (m, 2H, 2×ArH), 6.86 (dd, 1H, J=3.5 and 5.0 Hz, ArH), 6.79 (d, 1H, J=3.5 Hz, ArH), 5.03 (s, 2H, OCH₂), 4.54 (dd, 1H, J=5.8 and 3.5 Hz, H-5), 4.44 (d, 1H, J=3.5 Hz, H-4), 3.87 (d, 1H, J=15.2 Hz, CHHAr), 3.74 (d, 1H, J=15.2 Hz, CHHAr), 2.53 (d, 1H, J=10.8 Hz, H-6ₐₓ), 2.42 (dd, 1H, J=10.8 and 5.8 Hz, H-6_{eq}), 0.95 (s, 9H, C(CH₃)₃), 0.81 (s, 9H, C(CH₃)₃), 0.20 (s, 3H, CH₃), 0.18 (s, 3H, CH₃), 0.16 (s, 3H, CH₃) and 0.06 (s, 3H, CH₃) ppm. ¹³C NMR (63 MHz, CDCl₃) δ 175.2 (C), 148.2 (C), 142.7 (C), 140.0 (C), 139.9 (C), 139.1 (C), 129.7 (C), 126.4 (CH), 124.6 (CH), 124.3 (CH), 124.2 (CH), 123.6 (CH), 122.8 (CH), 122.6 (CH), 122.3 (CH), 74.6 (C), 74.5 (CH), 68.5 (CH₂), 67.3 (CH), 37.4 (CH₂), 25.6 (C(CH₃)₃), 24.7 (C(CH₃)₃), 18.0 (C(CH₃)₃), 17.9 (C(CH₃)₃), −3.4 (CH₃), −3.5 (CH₃), −4.5 (CH₃) and −4.6 (CH₃) ppm. IR (film) 1797 (C═O) cm⁻¹. MS (ESI) m/z (%) 665 (MNa⁺). HRMS calcd for C₃₃H₄₆O₅S₂Si₂Na(MNa⁺): 665.2217; found, 665.2225.

### Example 74

(1R,4S,5R)-1,4-dihydroxy-3-(benzo[b]thiophen-2-yl)methoxy-2-(thien-2-yl)methylcyclohex-2-en-1,5-carholactone (3e). The experimental procedure used was the same as for compound 3a using 15 mg (0.02 mmol) of ether 2c, 50 μL of TBAF and 0.4 mL of THF. Yield=7 mg (84%). White solid. [α]_{D}²⁰=−155″ (c1.2, in MeOH ). ¹H NMR (250 MHz, CD₃OD) δ 7.71 (m, 2H, 2×ArH), 7.25 (m, 3H, 3×ArH), 7.18 (s, 1H, ArH), 7.00 (dd, 1H, J=5.0 and 1.5 Hz, ArH), 6.74 (m, 1H, ArH), 5.27 (d, 1H, J=12.5 Hz, OCHH), 5.17 (d, 1H, J=12.5 Hz, OCHH), 4.55 (m, 1H, H-5), 4.47 (d, 1H, J=3.2 Hz, H-4), 3.78 (d, 1H, J=14.8 Hz, CHHAr), 3.63 (d, 1H, J=14.8 Hz, CHHAr) and 2.29 (m, 2H, H-6) ppm. ¹³C NMR (63 MHz, CD₃OD) δ 178.8 (C), 148.6 (C), 144.2 (C), 142.3 (C), 141.6 (C), 140.9 (C), 128.0 (C), 127.3 (CH), 126.3 (CH), 125.6 (CH), 125.4 (CH), 124.8 (CH), 124.1 (CH), 124.0 (CH), 123.3 (CH), 76.9 (CH), 73.7 (C), 66.8 (CH₂), 66.3 (CH), 38.4 (CH₂) and 24.9 (CH₂) ppm. IR (KBr) 3452 (O—H) and 1770 (C═O) cm⁻¹. MS (CI) m/z (%) 415 (MH⁺). HRMS calcd for C₂₁H₁₉O₅S₂ (MH): 415.0674; found, 415.0674.

### Example 75

Sodium (1R,4S,5R)-1,4,5-trihydroxy-3-(benzo[b]thiophen-5-yl)methoxy-2-(thien-2-yl)methylcyclohex-2-en-1-carboxylate (Ib-13). The same experimental procedure was the same as for compound Ib-6 (example 64), but using carbolactone 3e as starting material (20 mg, 0.05 mmol) in 0.5 mL of THF and 97 μL of NaOH (aq.). Yield=22 mg (99%). White solid. Mp: 184° C. (dec.). [α]_{D}²⁰=−24° (c1.5, in H₂O). ¹H NMR (250 MHz, 50% CD₃OD/D₂O) δ 7.82 (m, 1H, ArH), 7.77 (m, 1H, ArH), 7.35 (m, 2H, 2×ArH), 7.26 (s, 1H, ArH), 7.10 (dd, 1H, J 3.3 and 3.0 Hz, ArH), 6.82 (d, 2H, J 3.8 Hz, 2×ArH), 5.25 (d, 1H, J 12.0 Hz, CHHO), 5.04 (d, 1H, J 12.0 Hz, CHHO), 4.36 (d, 1H, J 5.2 Hz, H-4), 3.92 (m, 1H, H-5), 3.73 (d, 1H, J 15.3 Hz, CHHAr), 3.31 (m, 1H, J 15.3 Hz, CHHAr) and 2.10 (m, 2H, H-6) ppm. ¹³C NMR (63 MHz, 50% CD₃OD/D₂O) δ 180.8 (C), 152.0 (C), 145.4 (C), 142.7 (C), 141.5 (C), 140.9 (C), 127.1 (CH), 126.3 (CH), 125.4 (CH), 125.3 (CH), 124.7 (CH), 123.7 (2xCH+C), 123.3 (CH), 77.1 (C), 71.6 (CH), 70.0 (CH), 66.5 (CH₂), 37.4 (CH₂) and 27.2 (CH₂) ppm. IR (KBr) 3442 (O—H) and 1668 (C═O) cm⁻¹. MS (ESI) m/z (%) 431 (M⁻). HRMS calculated for C₂₁H₁₉O₆S₂ (M⁻): 431.0618; found, 431.0602.

### Example 76

Methyl (1R,4S,5R)-1,4,5-trihyclroxy-3-(benzo[b]thiophen-5-yl)methoxycyclohex-2-en-1-carboxylate (Ib-14). The experimental procedure used was the same as for compound Ib-15 using 52 mg (0.16 mmol) of lactone 3b, 10 mg (0.18 mmol) of NaOMe and 1.2 mL of methanol. Yield: 38 mg (68%). White solid. [α]_{D}²⁰=−31° (c1.4, in MeOH ). ¹H NMR (300 MHz, CD₃OD) δ 7.89 (d, 2H, J 8.4 Hz, 2×ArH), 7.58 (d, 1H, J5.7 Hz, ArH), 7.40 (dd, 1H, J8.4 and 1.2 Hz, ArH), 7.37 (d, 1H, J5.7 Hz, ArH), 4.98 (s, 1H, H-2), 4.94 (s, 2H, CH₂O), 4.02 (m, 2H, H-5+H-4), 3.70 (s, 3H, OCH₃) and 2.04 (m, 2H, H-6) ppm. ¹³C NMR (75 MHz, CD₃OD) δ 176.7 (C), 158.9 (C), 141.3 (C), 134.3 (C), 128.2 (C), 128.1 (CH), 125.3 (CH), 124.9 (CH), 123.9 (CH), 123.4 (CH), 99.5 (CH), 74.3 (C), 74.0 (CH), 71.0 (CH₂), 70.5 (CH), 53.1 (CH₃) and 40.3 (CH₂) ppm. IR (KBr) 3446 (O—H), 3305 (O—H) and 1732 (C═O) cm⁻¹. MS (ESI) 30 m/z (%) 373 (MNa⁻). HRMS calculated for C₁₇H₁₈O₆SNa (MNa⁺): 373.0716; found, 373.0711.

### Example 77

Methyl (1R,4S,5R)-3-(benzo[b]thiophen-5-yl)methoxy-1,4,5-tribinyroxycyclohex-2-en-1-carboxylate (6b). A solution of ester Ib-14 (15 mg, 0.04 mmol), pyridine (18 μL, 0.22 mmol) and butyryl chloride (23 μL, 0.22 mmol) in acetonitrile (1 mL) at 0° C. was stirred during 20 h during which time the reaction mixture reached room temperature. Ethyl acetate and water were added and the organic layer was separated. The aqueous phase was extracted with ethyl acetate (2×2 mL). All the combined organic extracts were dried Na₂SO₄ (anh.), filtered and evaporated. The obtained residue was purified by flash chromatography eluting with (30:70) diethyl ether/hexanes to yield ester 6b (18 mg, 80%) as yellow oil. [α]_{D}²⁰=+17° (c1.3, in CHCl₃). ¹H NMR (250 MHz, CDCk₃) δ 7.83 (d, 1H, J 8.2 Hz, ArH), 7.73 (s, 1H, ArH), 7.45 (d, 1H, J5.5 Hz, ArH), 7.28 (m, 2H, 2×ArH), 5.74 (d, 1H, J8.5 Hz, H-4), 5.45 (m, 2H, H-5+H-2), 4.96 (d, 1H, J 11.2 Hz, CHHO), 4.87 (d, 1H, J 11.2 Hz, CHHO), 3.73 (s, 3H, OCH₃), 2.50 (dd, 1H, J 13.0 and 3.4 Hz, H-6_{eq}), 2.33-2.23 (m, 7H, 3×CH₂₊H-6ₐₓ), 1.70-1.51 (m, 6H, 3×CH₂) and 0.96-0.77 (m, 9H, 3×CH₃) ppm. ¹³C NMR (63 MHz, CDCl₃) δ 172.9 (C), 172.8 (C), 172.6 (C), 171.0 (C), 155.3 (C), 139.6 (C), 139.3 (C), 131.9 (C), 127.0 (CH), 123.7 (CH), 123.6 (CH), 122.5 (CH), 122.4 (CH), 97.5 (CH), 77.4 (C), 70.1 (CH2), 69.9 (CH), 68.6 (CH), 52.9 (CH3), 36.1 (CH2), 36.0 (3×CH₂), 18.3 (3×CH₂) and 13.5 (3×CH₃) ppm. TR (film) 1747 (C═O) cm⁻¹.

### Example 78

Methyl (1R,4S,5R)-3-(benzo[b]thiophen-5-yl)methoxy-1,4,5-tripentyroxycyclohex-2-en-1-carboxylate (6c). The experimental procedure used was the same as for compound 6b using pentanoyl chloride. Yield: 83%. Yellow oil. [α]_{D}²⁰=+10° (c1.0, in CHCl₃). ¹H NMR (250 MHz, CDCl₃) δ 7.83 (d, 1H, J 8.3 Hz, ArH), 7.73 (br s, 1H, ArH), 7.46 (d, 1H, J5.5 Hz, ArH), 7.31 (d, 1H, J5.5 Hz, ArH), 7.26 (dd, 1H, J 8.2 and 1.5 Hz, ArH), 5.74 (d, 1H, J 8.5 and 1.3 Hz, H-4), 5.45 (m, 2H, H-5+H-2), 4.96 (d, 1H, J 11.5 Hz, CHHO), 4.86 (d, 1H, J 11.5 Hz, CHHO), 3.74 (s, 3H, OCH₃), 2.49 (dd, 1H, J 13.2 and 3.8 Hz, H-6_{eq}), 2.36-2.24 (m, 6H, 3×CH₂), 1.94 (dd, 1H, J 13.2 and 12.2 Hz, H-6ₐₓ), 1.62-1.43 (m, 6H, 3×CH₂) and 1.41-1.14 (m, 6H, 3×CH₂), 0.92 (m, 6H, 2×CH₃) and 0.57 (t, 3H, J 7.3 Hz, CH₃) ppm. ¹³C NMR (125 MHz, CDCl₃) δ 174.8 (C), 173.1 (C), 172.8 (C), 171.1 (C), 155.3 (C), 139.6 (C), 139.4 (C), 131.9 (C), 127.0 (CH), 123.8 (CH), 123.7 (CH), 122.6 (CH), 122.5 (CH), 97.5 (CH), 70.2 (C), 70.1 (CH₂), 70.0 (CH), 68.6 (CH), 52.9 (CH₃), 35.2 (CH₂), 34.0 (CH₂), 33.9 (CH₂), 33.8 (CH₂), 27.0 (2×CH₂), 26.8 (CH₂), 22.2 (2×CH₂), 22.1 (CH₂), 13.7 (2×CH₃) and 13.5 (CH₃) ppm. IR (film) 1747 (C=O) cm⁻¹.

### Example 79

Methyl (1R,4S,5R)-3-(benzo[b]thiophen-5-yl)methoxy-2-(benzo[b]thiophen-5-yl)methyl-1,4,5-tribttryroxycyclohex-2-en-1-carboxylate (6d). The experimental procedure used was the same as for compound 6b using ester Ib-15. Yield: 87%. Beige solid. Mp: 96-98° C. [α]_{D}²⁰=−57° (c1.0, in CHCl₃). ¹H NMR (250 MHz, CDCl3) δ 7.79 (d, 1H, J 8.3 Hz, ArH), 7.71 (d, 1H, J 8.7 Hz, ArH), 7.67 (s, 1H, ArH), 7.56 (s, 1H, ArH), 7.44 (d, 1H, J 5.2 Hz, ArH), 7.39 (d, 1H, J 5.0 Hz, ArH), 7.19 (m, 4H, 4×ArH), 6.07 (d, 1H, J7.5 Hz, H-4), 5.47 (m, 1H, H-5), 5.00 (d, 1H, J 11.5 Hz, CHHO), 4.79 (d, 1H, J 11.5 Hz, CHHO), 3.36 (d, 1H, J 15.0 Hz, CHHAr), 3.33 (d, 1H, J 15.0 Hz, CHHAr), 3.31 (s, 3H, CH₃O) 2.25 (m, 7H, 3×CH₂ and H-6ₐₓ), 2.09 (dd, 1H, J 12.7 and 4.0 Hz, H-6_{ec}), 1.66-1.54 (m, 6H, 3×CH₂) and 0.95-0.84 (m, 9H, 3×CH₃) ppm. ¹³C NMR (63 MHz, CDCl₃) δ 174.9 (2×C), 173.4 (C), 172.5 (C), 149.0 (C), 139.7 (C), 139.6 (C), 139.4 (C), 137.6 (C), 135.0 (C), 132.7 (C), 126.9 (CH), 126.4 (CH), 125.6 (CH), 124.1 (C+CH), 123.8 (CH), 123.7 (CH), 123.5 (CH), 122.9 (CH), 122.4 (CH), 122.1 (CH), 75.1 (C), 72.2 (CH₂), 69.9 (CH), 69.0 (CH), 52.9 (CH₃), 37.7 (CH₂), 36.1 (2×CH₂), 32.3 (CH₂), 29.6 (CH₂), 18.3 (3×CH₂) and 13.6 (3×CH₃) ppm. IR (film) 1730 (C═O) cm⁻¹.

### Example 80

Methyl (1R,4S,5R)-3-(benzo[b]thiophen-5-yl)methoxy-2-(benzo[b]thiophen-5-yl)methyl-1,4,5-tripentyroxycyclohex-2-en-1-carboxylate (6e). The experimental procedure used was the same as for compound 6c using ester Ib-14. Yield: 87%. White solid. Mp: 82-84° C. [α]_{D}²⁰=−48° (c1.0, in CHCl₃). ¹H NMR (250 MHz, CDCl₃) δ 7.79 (d, 1H, J 8.3 Hz, ArH), 7.71 (d, 1H, J 8.3 Hz, ArH), 7.67 (br s, 1H, ArH), 7.57 (br s, 1H, ArH), 7.44 (d, 1H, J 5.3 Hz, ArH), 7.39 (d, 1H, J 5.3 Hz, ArH), 7.25 (m, 3H, 3×ArH), 7.16 (dd, 1H, J 8.3 and 1.5 Hz, ArH), 6.07 (d, 1H, J 7.8 Hz, H-4), 5.47 (m, 1H, H-5), 5.00 (d, 1H, J 11.3 Hz, CHHO), 4.79 (d, 1H, J 11.3 Hz, CHHO), 3.63 (m, 1H, J 15.2 Hz, CHHAr), 3.37 (d, 1H, J 15.2 Hz, CHHAr), 3.31 (s, 3H, OCH₃), 2.26 (m, 7H, 3×CH2+H-6ₐₓ), 2.09 (dd, 1H, J 12.8 and 4.0 Hz, H-6_{eq}), 1.60-1.47 (m, 6H, 3×CH₂), 1.36-1.20 (m, 6H, 3×CH₂) and 0.93-0.79 (m, 9H, 3×CH₃) ppm. ¹³C NMR (63 MHz, CDCl₃) δ 174.9 (2×C), 173.6 (C), 172.6 (C), 149.1 (C), 139.7 (C), 139.6 (C), 139.4 (C), 137.6 (C), 135.0 (C), 132.8 (C), 126.9 (CH), 126.5 (CH), 125.6 (CH), 124.1 (C), 124.1 (CH), 123.8 (CH), 123.7 (CH), 123.5 (CH), 122.8 (CH), 122.4 (CH), 122.1 (CH), 75.1 (C), 72.2 (CH₂), 69.9 (CH), 69.0 (CH), 52.9 (CH₃), 37.7 (CH₂), 34.0 (2×CH₂), 32.3 (CH₂), 29.7 (CH₂), 26.9 (3×CH₂), 22.2 (3×CH₂) and 13.7 (3×CH₃) ppm. IR (KBr) 1726 (C═O) cm⁻¹.

### Example 81

(4R,6R,7S)-4,7-dfftert-butyldimethylsilyloxy)-2-(1-phenylvinyl)-4,5,6,7-tetrahydrobenzo[b]thiophen-4,6-carbolactone (7). A Shlenck tube was charged with iodide VII of example 37 (190 mg, 0.34 mmol), Pd(PPh₃)₄ (40 mg, 0.03 mmol) and dry dioxane (3.4 mL). Anhydrous K₂CO₃ (0.92 mL, 1.01 mmol, 1.1 M) and 1-phenylvinylboronic acid pinacol ester (116 mg, 0.50 mmol) was then added and the resultant solution was deoxygenated and heated at 100° C. for 3 h. After cooling to room temperature, the reaction mixture was filtered through a plug of Celite and the precipitate was washed with hexane. The filtrate and the washings were concentrated and the obtained residue was purified by columm chromatography, previously neutralized with 5% triethylamine-hexanes, eluting with diethyl ether-hexanes (5:95) to yield thiophene 7 (105 mg, 58%) as a light yellow oil. [α]_{D}²⁰=−46.6° (c1.9, in CHCl₃). ¹H NMR (250 MHz, CDCl₃) δ 6.88-6.85 (m, 5H, 5×ArH), 6.86 (s, 1H, H-2), 5.56 (s, 1H), 5.26 (s, 1H), 4.86 (d, J=3.2 Hz, 1H, H-7), 4.67 (dd, J=5.6 and 3.2 Hz, 1H, H-6), 2.61 (d, J=10.9 Hz, 1H, H-5ₐₓ), 2.51 (dd, J=10.9 and 5.8 Hz, 1H, H-5_{eq}), 0.94 (s, 9H, C(CH₃)₃), 0.89 (s, 9H, C(CH₃)₃), 0.23 (s, 3H, CH₃), 0.21 (s, 3H, CH₃), 0.20 (s, 3H, CH₃) and 0.11 (s, 3H, CH,) ppm. ¹³C NMR (63 MHz, CDCl₃) δ 175.1 (C), 145.3 (C), 143.1 (C), 141.7 (C), 140.2 (C), 134.0 (C), 128.2 (2×CH), 128.1 (3×CH), 122.4 (CH), 114.0 (CH₂), 77.2 (CH), 75.3 (C), 66.3 (CH), 37.8 (CH₂), 25.7 (C(CH₃)₃), 25.5 (C(CH₃)₃), 18.1 (C(CH₃)₃), 18.0 (C(CH₃)₃), −3.0 (CH₃), —3.2 (CH₃), −4.6 (CH₃) and −4.6 (CH₃) ppm. IR (film) 1803 (C═O) cm⁻. MS (ESI) m/z (%) 543 (MH⁺). HRMS calcd for C₂₉H₄₃O₄SSi₂ (MH⁺): 543.2415; found, 543.2412.

### Example 82

(4R,6R,7S)-4,7-clihydroxy-2-(1-phenylvinyl)-4,5,6,7-tetrahydro-benzo[b]thiophen-4,6-carbolactone (8). The experimental procedure used was the same as for compound V-2 of example 32, but using silyl ether 7 as starting material (140 mg, 0.26 mmol) in 3.4 mL of THF and 0.65 mL de tetrabutylammonium fluoride (0.65 mmol). Yield: 68 mg (84%). Colourless oil. [α]_{D}²⁰=−37.7° (c1.2, in CH₃OH). ¹H NMR (250 MHz, CD₃OD) δ 7.48-7.28 (m, 5H, 5×ArH), 6.90 (s, 1H, H-3), 5.59 (s, 1H), 5.25 (s, 1H), 4.82 (m, 2H, H-6+H-7) and 2.52 (m, 2H, H-8) ppm. ¹³C NMR (63 MHz, CD₃OD) δ 178.2 (C), 147.1 (C), 145.0 (C), 142.6 (C), 141.8 (C), 136.1 (C), 129.4 (3×CH), 129.3 (2×CH), 122.6 (CH), 114.6 (CH₂), 79.1 (CH), 74.8 (C) 66.7 (CH) and 38.4 (CH₂) ppm. IR (film) 3408 (O—H) and 1778 (C═O) cm'. MS (ESI) m/z (%) 315 (MH⁻). HRMS calculated for C₁₂H₁₅O₄S (MH): 315.0686; found, 315.0684.

### Example 83

(4R,6R,7S)-4,6,7-trihydroxy-2-(1-phenylvinyl)-4,5,6,7-tetrahydro-benzo[b]thiophen-4-carboxylic acid (Ic-10). The experimental procedure used was the same as for compound Ic-1 of example 33, but using lactone 8 as starting material (12 mg, 0.04 mmol) in 0.2 mL of THF and 0.1 mL of LiOH (aq.). Yield: 12 mg (92%). Orange solid. [α]_{D}²⁰=4.9° (c1.0, in CH₃OH). ¹H NMR (250 MHz, D₂O) δ 7.35 (m, 5H, 5×ArH), 6.67 (s, 1H, H-3), 5.54 (s, 1H, CHH), 5.19 (s, 1H, CHH), 4.67 (d, J8.3 Hz, 1H, H-7), 4.14-3.96 (m, 1H, H-6) and 2.31 (m, 2H, CH₂) ppm. ¹³C NMR (63 MHz, D₂O) δ 179.4 (C), 145.5 (C), 143.1 (C), 141.7 (C), 140.5 (C), 137.4 (C), 128.8 (3×CH), 128.5 (2×CH), 124.3 (CH), 114.6 (CH₂), 74.0 (C), 72.3 (CH), 70.6 (CH) and 41.3 (CH₂) ppm. IR (KBr) 3390 (O—H) and 1600 (C═O) cm⁻¹. MS (ESI) m/z (%): 331 (M−H). HRMS calcd for C₁₇H₁₅O₅S (M−H): 331.0636 found, 331.0637.

### Example 84

(4R,6R,7S)-4,7-di(tert-butyldinzethylsilyloxy-2-styryl-4,5,6,7-tetrahydro-benzo[b]thiophen-4,6-carbolactone (10). The experimental procedure used was the same as for compound 7 (example 81), but using iodide VII of example 37 as starting material (150 mg, 0.26 mmol), Pd(PPh₃)₄ (19 mg, 0.02 mmol), K₂CO₃ (0.72 mL) and (E)-4,4,5,5-tetramethyl-2-styryl-1,3,2-dioxaborolane (91 mg, 0.40 mmol) in 2.65 mL of dioxanc. Yield: 94 mg (65%). Colourless oil. [α]_{D}²⁰=−21.2° (c1.0, in CHCl₃). ¹H NMR (250 MHz, CDCl₃) δ 7.50 (m, 2H, 2×ArH), 7.38 (m, 2H, 2×ArH), 7.30 (m, 1H, ArH), 7.20 (d, J=16.0 Hz, 1H, CH═CHAr), 7.03 (s, 1H, H-3), 6.91 (d, J=16.0 Hz, 1H, CH═CHAr), 4.90 (d, J=3.0 Hz, 1H, H-7), 4.71 (dd, J=5.5 and 3.0 Hz, 1H, H-6), 2.66 (d, J=11.0 Hz, H-5ₐₓ), 2.56 (ddd, J=11.0, 5.5, and 5.0 Hz, 1H, H-5_{eq}), 1.05 (s, 9H, C(CH₃)₃), 0.99 (s, 9H, C(CH₃)₃), 0.32 (s, 3H, CH₃), 0.30 (s, 3H, CH₃), 0.26 (s, 3H, CH3) and 0.21 (s, 3H, CH₃) ppm. ¹³C NMR (63 MHz, CDCl₃) δ 175.1 (C), 143.5 (C), 142.1 (C), 136.6 (C), 133.2 (C), 129.1 (CH), 128.7 (2xCH), 127.8 (CH), 126.4 (2×CH), 121.8 (CH), 121.5 (CH), 77.2 (CH), 75.4 (C), 66.3 (CH), 37.7 (CH₂), 25.7 (2×C(CH₃)₃), 18.2 (C(CH₃)₃), 18.0 (C(CH₃)₃), −3.0 (CH₃), −3.2 (CH₃), −4.6 (CH₃) and 4.6 (CH₃) ppm. IR (film) 1801 (C═O) cm⁻¹. MS (ESI) m/z (%) 543 (MH⁺). HRMS calculated for C₂₉H₄₃O₄SSi₂ (MH⁺): 543.2415; found, 543.2422.

### Example 85

(4R,6R,7S)-4,7-dihydroxy-2-styryl-4,5,6,7-tetrahydrobenzo[b]thiophen-4,6-carbolactone (11). The experimental procedure used was the same as for compound V-2 of example 32, but using silyl ether 10 as starting material (40 mg, 0.07 mmol) in 1.0 ml. of THF and 0.18 mL de tetrabutylammonium fluoride (0.18 mmol). Yield: 20 mg (87%). White solid. Mp: 196-200° C. (dec.). [α]_{D}²⁰=12.2° (c1.7, in MeOH ). ¹H NMR (250 MHz, CD₃OD) δ 7.50 (m, 2H, 2×ArH), 7.33 (m, 3H, 2×ArH+CH═CHAr), 7.25 (m, 1H, ArH), 7.10 (s, 1H, H-3), 6.93 (d, J=16.0 Hz, 1H, CH═CHAr), 4.84-4.80 (m, 2H, H-7−H-6), 2.53 (m, 2H, H-5) ppm. ¹³C NMR (63 MHz, CD₃OD) δ 178.2 (C), 145.4 (C), 142.9 (C), 138.1 (C), 135.1 (C), 130.3 (CH), 129.8 (2×CH), 129.0 (CH), 127.5 (2×CH), 122.6 (CH), 122.3 (CH), 79.1 (CH), 74.9 (C), 66.8 (CH), 38.4 (CH₂) ppm. IR (KBr) 3490 (O—H), 3305 (O—H), 1782 and 1756 (C═O) cm⁻¹.

### Example 86

(4R,6R,7S)-4,7-dihydroxy-2-styryl-4,5,6,7-tetrahydrobenzo[b]thiophen-4-carboxylic acid (Ic-11). The experimental procedure used was the same as for compound Ic-1 of example 33, but using lactone 11 as starting material (15 mg, 0.05 mmol) in 0.24 mL of THF and 0.14 mL of LiOH (aq.). Yield: 14 mg (87%). White solid. Mp: 200° C. (dec.). [α]_{S}²⁰=50.5° (c1.0, in MeOH ). ¹H NMR (250 MHz, D₂O) δ 7.54 (m, 2H, 2×ArH), 7.41 (m, 2H, 2×ArH), 7.32 (m, 1H, ArH), 7.29 (d, J=16.3 Hz, 1H, CH═CHAr), 6.97 (d, J=16.3 Hz, 1H, CH═CHAr), 6.81 (s, 1H, H-3), 4.70 (d, J=8.0 Hz, 1H, H-7), 4.06 (m, 1H, H-6), 2.37 (m, 1H, H-5ₐₓ) and 2.18 (m, 1H, H-5_{eq}) ppm. ¹³C NMR (63 MHz, D₂O) δ 180.6 (C), 143.7 (C), 140.6 (C), 138.6 (C), 137.0 (C), 129.3 (2×CH), 128.6 (CH), 128.3 (CH), 126.7 (2×CH), 124.5 (CH), 122.2 (CH), 74.3 (C), 72.4 (CH), 70.9 (CH) and 41.5 (CH₂) ppm. IR (KBr) 3398 (O—H) and 1602 (C═O) cm⁻¹. MS (ESI) m/z (%): 331 (M−H). HRMS calculated for C₁₇H₁₅O₅S (M−H): 331.0635; found, 331.0637.

### Example 87

(4R,6R,7S)-4,7-dihydroxy-2-propyl-4,5,6,7-tetrahydrobenzo[b]thiophen-4,6-carbolactone (13). A suspension of thiophene V-8 of example 42 (76 mg, 0.30 mmol) and Pd(OH)₂ (15 mg) in methanol (4.3 mL) was stirred under hydrogen atmosphere at room temperature for 48 h. The mixture was filtered over Celite and the residue was washed with methanol. The filtrate and washings were evaporated to yield thiophene 13 (74 mg, 97%) as a white solid. Mp: 120-125° C. [α]_{D}²⁰=−75.2° (c1.5, in MeOH ). ¹H NMR (250 MHz, CD₃OD) δ 6.75 (s, 1H, H-3), 4.72 (m, 2H, H-6+H-7), 2.71 (t, J 7.3 Hz, 2H, CH₂Ar), 2.45 (m, 2H, H-5), 1.62 (m, 2H, CH₂CH₃) and 0.91 (t, J 7.3 Hz, 3H, CH₃) ppm. ¹³C NMR (75 MHz, CD₃OD) δ 178.4 (C), 148.3 (C), 141.9 (C), 133.6 (C), 120.6 (CH), 79.1 (CH), 74.9 (C), 66.6 (CH), 38.4 (CH₂), 33.1 (CH₂), 25.9 (CH₂) and 13.9 (CH₃) ppm. IR (KBr): 3409 (O—H) and 1779 (C═O) cm⁻¹.

### Example 88

(4R,6R,7S)-4,7-dihydroxy-2-(2-phenyl)ethyl-4,5,6,7-tetrahydro-benzo[b]thiophen-4,6-carholactone (14). The experimental procedure used was the same as for compound 13, but using lactone 11 as starting material (77 mg, 0.24 mmol) in 3.5 mL of MeOH and 15 mg of Pd(OH)₂. White solid. Mp: 85-97° C. [α]_{D}²⁰=−65.9° (c1.7, in MeOH). ¹H NMR (250 MHz, CD₃OD) δ 7.32-7.10 (m, 5H, 5×ArH), 6.82 (s, 1H, H-3), 4.78 (m, 2H, H-6+H-7), 3.09 (m, 2H, CH₂CH₂Ph), 2.93 (m, 2H, CH₂Ph) and 2.50 (m, 2H, H-5) ppm. ¹³C NMR (75 MHz, CD₃OD) δ 178.4 (C), 147.6 (C), 142.1 (C), 141.9 (C), 133.9 (C), 129.5 (4×CH), 127.2 (CH), 121.0 (CH), 79.2 (CH), 75.0 (C), 66.7 (CH), 38.9 (CH₂), 38.5 (CH₂) and 33.1 (CH₂) ppm. IR (KBr): 3409 (O—H) and 1776 (C═O) cm⁻¹. MS (ESI) m/z (%) 339 (MNa⁻). HRMS calculated for C₁₇H₁₆NaO₄S (MNa⁺): 339.0662; found, 339.0667.

### Example 89

(4R,6R,7S)-4,6,7-trihydroxy-2-phenethyl-4,5,6,7-tetrahydrobenzo[b]thiophene-4-carboxylic acid (Ic-12). The experimental procedure used was the same as for compound Ic-1 of example 33, but using lactone 14 as starting material (20 mg, 0.06 mmol) in 0.3 mL of THF and 0.19 mL of LiOH (aq.). White solid. Mp: ¹H NMR (250 MHz, CD₃OD) δ 7.29-7.07 (m, 5H, 5×ArH), 6.54 (s, 1H, H-3), 4.50 (d, 1H, J 6.7 Hz, H-6), 3.97 (m, 1H, H-7), 2.98 (m, 2H, CH₂CH₂Ph), 2.88 (m, 2H, CH₂CH₂Ph), 2.33 (dd, 1H, J 13.5 and 9.7 Hz, H-5ax) and 2.14 (dd, 1H, J 13.5 and 3.4 Hz, H-5eq) ppm. ¹³C NMR (75 MHz, CD₃OD) δ 179.4 (C), 146.1 (C), 142.4 (C), 140.4 (C), 138.9 (C), 129.5 (2×CH), 129.4 (2×CH), 123.7 (CH), 74.4 (C), 72.9 (C), 72.3 (CH), 41.3 (CH₂), 38.9 (CH₂) and 33.3 (CH₂) ppm. MS (ESI) m/z (%) 333 (M−H). HRMS calculated for C₁₇H₁₇O₅S (M−H): 333.0791; found, 333.0792.

### Example 90

(4R,6R,7S)-4,6,7-trihydroxy-2-propyl-4,5,6,7-tetrahyclrobenzo[b]thiophene-4-carboxylic acid (Ic-13). The experimental procedure used was the same as for compound Ic-1 of example 33, but using lactone 13 as starting material (20 mg, 0.08 mmol) in 0.4 mL of THF and 0.24 mL of LiOH (aq.). Yield: 21 mg (96%). White solid. ¹H NMR (250 MHz, CD₃OD) δ 6.47 (s, 1H, H-3), 4.46 (d, 1H, J 5.9 Hz, H-6), 3.91 (m, 1H, H-7), 2.61 (t, 2H, J7.3 Hz, CH₂), 2.40-2.00 (m, 2H, CH₂), 1.56 (m, 2H, CH₂) and 0.86 (t, 3H, J 7.3 Hz, CH₃) ppm. ¹³C NMR (75 MHz, CD₃OD) δ 179.5 (C), 146.8 (C), 140.1 (C), 139.0 (C), 123.5 (CH), 74.6 (C), 72.8 (CH), 72.3 (CH), 41.2 (CH₂), 33.3 (CH₂), 26.0 (CH₂) and 14.0 (CH₃) ppm. MS (ESI) m/z (%) 271 (M−H). HRMS calculated for C₁₂H₁₅O₅S (M−H): 271.0635; found, 271.0630.

### Example 91

(4R,6R,7S)-2-Ethyl-4,6,7-trihydroxy-4,5,6,7-tetrahydrobenzo[b]tiophene-4-carboxylic acid (Ic-14). The experimental procedure used was the same as for compound V-15 (example 52) but utilizing acid Ic-3 (18 mg, 0.070 mmol), 4 mg of Pd—C (10%) in methanol (0.7 mL). Yield=13.9 mg (77%). Beige solid. Mp: 130° C. (dcc.). [α]_{D}²⁰−34.6° (c1.0, in MeOH ). ¹H NMR (250 MHz, D₂O) δ 6.40 (br s, 1H, H-3), 4.55 (d, 1H, J=7.0 Hz, H-7), 3.91 (m, 1H, H-6), 2.66 (q, 2H, J=7.3 Hz, CH₂CH₃), 2.20 (m, 1H, H-5ₐₓ), 2.04 (m, 1H, H-5_{eq}) and 1.12 (t, 3H, J=7.3 Hz, CH₃) ppm. ¹C RMN (63 MHz, D₂O) δ 180.8 (C), 149.6 (C), 138.4 (C), 137.6 (C), 121.2 (CH), 74.3 (C), 72.3 (CH), 71.0 (CH), 41.4 (CH₂), 23.4 (CH₂) and 15.4 (CH₃) ppm. IR (KBr): 3435 (O—H) and 1724 (C═O) cm⁻¹. MS (ESI) m/z (%) 257 (M−H); HRMS calcd for C₁₁H₁₃O₅S (M−H): 257.0478; found, 257.0469.

### Example 92

(4R,6R,7S)-2-benzyl-4,7-di(tert-butyldimethylsilyloxy)-4,5,6,7-tetrahydro-benzo[b]thiophen-4,6-carholactone (20). A Shlenck tube was charged with iodide VII (example 37) (234 mg, 0.41 mmol), Pd(dppf)Cl₂.CH₂Cl₂ (35 mg, 0.04 mmol) and dry dioxane (4.1 mL). K₂CO₃ (1.12 mL, 1.23 mmol, 1.1 M) and 2-benzyl-4,4,5,5-tetramethyl-1,3,2-dioxaborolane (120 μL, 0.54 mmol) was then added and the resultant solution was deoxygenated and heated at 90° C. for 9 h. After cooling to room temperature, the reaction mixture was filtered through a plug of Celite and the precipitate was washed with hexane. The filtrate and the washings were concentrated. The obtained residue was purified by columm chromatography over silica gel eluting with diethyl ether-hexanes (2:98) to afford benzyl tiophene 20 (60 mg, 28%) and thiophene V-1 (42 mg, 22%). Colourless oil. [α]_{D}²⁰=−7.7 (c1.3, in CHCl₃). ¹H NMR (250 MHz, CDCl₃) δ 7.12 (m, 5H, 5×ArH), 6.56 (s, 1H, H-3), 4.63 (d, J=3.1 Hz, 1H, H-7), 4.47 (dd, J=5.9 and 3.1 Hz, 1H, H-6), 3.96 (d, J=16.1 Hz, CHHPh), 3.88 (d, J=16.1 Hz, CHHPh), 2.42 (d, J=10.9 Hz, 1H, H-5_{eq}), 2.31 (dd, J=10.9 and 5.9 Hz, 1H, H-5ₐₓ), 0.78 (s, 9H, C(CH₃)₃), 0.74 (s, 9H, C(CH₃)₃), 0.06 (s, 3H, CH₃), 0.01 (3H, CH₃), 0.00 (s, 3H, CH₃) and −0.05 (s, 3H, CH₃) ppm. ¹³C NMR (63 MHz, CDCl₃) δ 175.3 (C), 145.2 (C), 141.3 (C), 139.5 (C), 133.1 (C), 128.6 (2×CH), 128.5 (2×CH), 126.6 (CH), 121.2 (CH), 77.3 (CH), 75.5 (C), 66.3 (CH), 37.8 (CH₂), 36.4 (CH₂), 25.7 (C(CH₃)₃), 25.6 (C(CH₃)₃), 18.2 (C(CH₃)₃), 18.0 (C(CH₃)₃), −3.0 (CH₃), −3.2 (CH₃), −4.6 (CH₃) and −4.7 (CH₃) ppm. IR (film) 1801 (C═O) cm⁻¹. MS (CI) m/z (%) 531 (MH⁺). HRMS calcd for C₂₈H₄₃O₄SSi₂ (MH⁺): 531.2421; found, 531.2438.

### Example 93

(4R,6R,7S)-2-benzyl-4,7-dihydroxy-4,5,6,7-tetrahydrobenzo[b]thiophen-4,6-carbolactone (21). The experimental procedure used was the same as for compound V-2 (example 32), but using silyl ether 20 (57 mg, 0.11 mmol) in 1.5 mL of THF and 0.27 mL de tetrabutylammonium fluoride (0.27 mmol). Yield: 29 mg (91%). White solid. Mp: 116-118° C. [α]_{D}²⁰=−77.7 (c2.2, in CHCl₃). ¹H NMR (250 MHz, CD₃OD) δ 7.33-7.15 (m, 5H, 5×ArH), 6.83 (s, 1H, H-3), 4.83-4.66 (m, 2H, H-7+H-6), 4.10 (s, 2H, CH₂Ph) and 2.48 (m, 2H, H-5) ppm. ¹³C NMR (63 MHz, CD₃OD) δ 178.4 (C), 147.6 (C), 142.1 (C), 141.4 (C), 134.7 (C), 129.7 (2×CH), 129.6 (2×CH), 127.7 (CH), 121.5 (CH), 79.1 (CH), 74.9 (C), 66.7 (CH), 38.5 (CH₂) and 37.2 (CH₂) ppm. IR (film) 3411 (O—H) and 1776 (C═O) cm⁻¹. MS (ESI) m/z (%) 325 (MNa⁺). HRMS calcd for C₁₆H₁₄O₄SNa (MNa⁺): 325.0505; found, 325.0495.

### Example 94

(4R,6R,7S)-2-benzyl-4,6,7-trihydroxy-2-benzyl-4,5,6,7-tetrahydro-benzo[b]thiophen-4-carboxylic acid (Ic-15). The experimental procedure used was the same as for compound Ic-1 (example 33), using lactone 21 (24 mg, 0.08 mmol) in 0.34 mL of THE and 0.11 mL of LiOH (aq.). Yield: 24 mg (96%). White solid. Mp: 209-212° C. [α]_{D}²⁰=−14.6 (c2.0, in MeOH ). ¹H NMR (250 MHz, CD₃OD) δ 7.35-7.10 (m, 5H, 5×ArH), 6.61 (s, 1H, H-3), 4.54 (d, J=5.9 Hz, 1H, H-7), 4.04 (s, 2H, CH₂Ph), 4.00 (m, 1H, H-6), 2.34 (dd, J=13.7 and 8.4 Hz, H-5ₐₓ) and 2.17 (dd, J=13.7 and 3.3 Hz, H-5_{eq}) ppm. ¹³C NMR (63 MHz, CD₃OD) δ 180.8 (C), 145.7 (C), 141.7 (C), 140.4 (C), 140.2 (C), 129.7 (2×CH), 129.5 (2×CH), 127.4 (CH), 124.4 (CH), 74.6 (C), 72.6 (CH), 72.1 (CH), 40.4 (CH₂) and 37.3 (CH₂) ppm. IR (film) 3419 (O—H) and 1604 (C═O) cm¹. MS (ESI) m/z (%) 319 (M−H). HRMS calcd for C₁₆H₁₅O₅S (M−H): 319.0635; found, 319.0631.

### Example 95

(4R,6R,7R)-1-(2-(2-bromo)phenyl)ethyl-4,7-di(tert-butyldimethyl-silyloxy)-4,5,6,7-tetrahydro-1H-indole-4,6-carbolacame (22). A solution of allylketone III-1 (example 2) (500 mg, 1.14 mmol) in dicloromethane (50 mL) was placed into a gas-washing bottle and cooled down to −78° C. in a dry ice bath. The mixture was then flushed with argon for 5 min, oxygen for 5 min and then with an oxygen/ozone mixture for 12 min The end of the reaction was indicated by a 5% potassium iodide/water solution turning orange, which was located at the exit. Afterwards, the reaction mixture was flushed for 10 min with argon to remove excess ozone. The gas-washing bottle was taken from the cooling bath and the content was slowly warming up to room temperature. The solvent was evaporated under reduced pressure to afford an oil (500 mg) which was dissolved in a 1:1 mixture of dichloromethane-acetic acid (2 mL). The resultant solution was treated at 0° C. with 2-(2-bromo)phenylethylamine (0.18 mL, 1.25 mmol) and it was stirred for 30 min The reaction mixture was neutralized with powdered sodium bicarbonate and it was diluted with dichloromethane and water. The organic layer was separated and the aqueous layer was extracted twice with dichloromethane. All the combined organic extracts were dried (anh. Na₂SO₄), filtered and concentrated under reduced pressure. The obtained residue was purified by columm chromatography over silica gel, previously neutralized with triethylamine-hexanes (5:95), using diethyl ether-hexanes (5:95) as cluent to afford compound 22 (150 mg, 23%) as a colourless oil. [α]_{D}²⁰=−109.0 (c1.3, in CHCl₃). ¹H NMR (250 MHz, CDCl₃) δ 7.60-7.50 (m, 1H, ArH), 7.15-7.07 (m, 2H, 2×ArH), 6.60-6.55 (m, 1H, ArH), 6.50 (d, J=2.7 Hz, 1H, H-2), 6.14 (d, J=2.7 Hz, 1H, H-3), 4.50 (dd, J=5.5 and 3.5 Hz, 1H, H-6), 4.18 (d, J=3.5 Hz, 1H, H-7), 4.07 (m, 2H, NCH₂), 3.29-3.19 (m, 1H, CHHAr), 3.09-2.93 (m, 1H, CHHAr), 2.47 (d, J=10.5 Hz, H-5_{eq}), 2.38 (dd, J=10.5 and 5.5 Hz, H-5ₐₓ), 0.97 (s, 9H, C(CH₃)₃), 0.88 (s, 9H, C(CH₃), 0.22 (s, 3H, CH₃), 0.18 (s, 3H, CH₃), 0.14 (s, 3H, CH₃) and 0.14 (s, 3H, CH₃) ppm. ¹³C NMR (63 MHz, CDCl₃) δ 175.9 (C), 137.2 (C), 132.6 (CH), 130.8 (CH), 128.7 (CH), 127.9 (CH), 124.7 (C), 123.8 (C), 123.4 (C), 121.2 (CH), 103.5 (CH), 76.5 (CH), 74.2 (C), 64.1 (CH), 46.5 (CH₂), 38.3 (CH₂), 38.2 (CH₂), 25.7 (2×C(CH₃)₃), 18.2 (C(CH₃)₃), 18.0 (C(CH₃)₃), −2.9 (CH₃), −3.0 (CH₃), −4.1 (CH₃) and −4.3 (CH₃) ppm. IR (KBr) 1799 (C═O) cm⁻¹. MS (CI) m/z (%) 606 and 608 (MH⁻). HRMS calcd for C₂₉H₄₅O₄NSi₂⁸¹Br (MH⁺): 608.2050; found, 608.2043.

### Example 96

(8R,9R,11R)-8,11-di(tert-butyldimethylsilyloxy)-5,6,8,9,10,11-hexahydroindolo[2,1-a]isoquinoline-9,11-carbolactone (23). A Shlenck tube was charged with bromide 22 (150 mg, 0.25 mmol), PdCl₂(PPh₃)₂ (68 mg, 0.10 mmol), triphenylphosphine (51 mg, 0.19 mmol), K₂CO₃ (73 mg, 0.53 mmol) and dry DMF (1.5 mL). The resultant solution was deoxygenated and heated at 125° C. for 16 h. After cooling to room temperature, the solvent was removed under reduced pressure and the resultant residue was dissolved in diethyl ether and brine. The organic layer was separated and the aqueous phase was extracted with diethyl ether (×2). All the combined organic extracts were dried (anh. Na₂SO₄), ficltcred and concentrated under reduced pressure. The obtained residue was purified by columm chromatography over silica gel, previously neutralized with tricthylamine-hexanes (5:95), using diethyl ether-hexanes (5:95) as eluent to afford compound 23 (70 mg, 54%) together with 38 mg (25%) of starting material. Corrected yield=72%. Colourless oil. [α]_{D}²⁰=−25.3 (c1.2, in CHCl₃). ¹H NMR (250 MHz, CDCl₃) δ 7.32 (d, J=7.7 Hz, 1H, ArH), 7.08-6.89 (m, 3H, 3×ArH), 6.32 (s 1H, H-3), 4.69 (d, J=3.2 Hz, 1H, H-7), 4.51 (dd, J=5.7 and 3.2 Hz, 1H, H-6), 3.77 (t, J=6.5 Hz, 2H, NCH₂), 2.84 (t, J=6.5 Hz, 2H, ArCH₂), 2.42 (d, J=10.7 Hz, 1H, H-5_{eq}), 2.32 (dd, J=10.7 and 5.7 Hz, 1H, H-5ₐₓ), 0.84 (s, 9H, C(CH₃)₃), 0.74 (s, 9H, C(CH₃)₃), 0.10 (s, 3H, CH₃), 0.07 (s, 3H, CH₃), 0.05 (s, 3H, CH₃) and 0.01 (s, 3H, CH₃) ppm. ¹³C NMR (63 MHz, CDCl₃) δ 175.9 (C), 130.6 (C), 130.2 (C), 128.9 (C), 127.8 (CH), 127.2 (CH), 126.1 (CH), 124.5 (C), 124.4 (C), 122.7 (CH), 98.5 (CH), 76.7 (CH), 74.2 (C), 64.3 (CH), 41.4 (CH₂), 38.5 (CH₂), 28.9 (CH₂), 25.7 (C(CH₃)₃), 25.6 (C(CH₃)₃), 18.3 (C(CH₃)₃), 17.9 (C(CH₃)₃), −2.9 (CH₃), −3.0 (CH₃), −3.7 (CH₃) and −4.5 (CH₃) ppm. IR (KBr) 1795 (C═O) cm⁻¹. MS (ES1) m/z (%) 526 (MH⁺). HRMS calcd for C₂₉H₄₄O₄NSi₂ (MH⁺): 526.2803; found, 526.2799.

### Example 97

(8R,9R,11R)-8,11-dihydroxy-5,6,8,9,10,11-hexahydroindolo[2,1-a]isoquinoline-9,11-carbolactone (24). The experimental procedure used was the same as for compound V-2 (example 32), but using silyl ether 23 (50 mg, 0.10 mmol) in 1.3 mL of THF and 0.19 nth de tetrabutylammonium fluoride (0.19 mmol). Yield: 24 mg (86%). White solid. Mp: 157-161° C. [α]_{D}²⁰=−18.7 (c1.8, in MeOH ). ¹H NMR (400 MHz, CD₃OD) δ 7.48 (d, J=7.6 Hz, 1H, ArH), 7.22-7.15 (m, 2H, 2×ArH), 7.11-7.06 (m, 1H, ArH), 6.53 (s, 1H, H-3), 4.80 (d, J=3.2 Hz, 1H, H-7), 4.79-4.76 (m, 1H, H-6), 4.06-4.02 (m, 2H, NCH₂), 3.08-2.93 (m, 2H, ArCH₂), 2.53 (d, J=11.2 Hz, 1H, H-5_{eq}) and 2.48 (dd, J=11.2 and 5.2 Hz, H-5ₐₓ) ppm. ¹³C NMR (100 MHz, CD₃OD) δ 179.2 (C), 132.3 (C), 132.2 (C), 130.3 (C), 129.1 (CH), 128.2 (CH), 127.3 (CH), 126.1 (C), 124.6 (C), 123.5 (CH), 98.7 (CH), 79.2 (CH), 73.8 (C), 64.2 (CH), 42.5 (CH₂), 39.3 (CH₂) and 29.9 (CH₂) ppm. IR (KBr) 3400 (O—H) and 1765 (C═O) cm⁻¹. MS (ESI) m/z (%) 298 (MH⁺). HRMS calcd for C₁₇H₁₆O₄N (MH'): 298.1074; found, 298.1075.

### Example 98

Ethyl (4R,6R,7S)-4,6,7-trihydroxy-2-phenethyl-4,5,6,7-tetrahydro-benzo[b]thiophen-4-carboxylate (Ic-14). A solution of the lactone 14 (example 88) (20 mg, 0.06 mmol) in dry ethanol (0.6 mL) was treated with sodium ethoxidc (4 mg, 0.06 mmol). The resultant mixture was stirred at room temperature for 35 min and the solvent was then evaporated under reduced pressure. The obtained residue was dissolved in ethyl acetate and water. The organic layer was separated and the aqueous phase was extracted with ethyl acetate (×2). All the combined organic extracts were dried (anh. Na₂SO₄), filtered and concentrated under reduced pressure. The obtained residue was purified by flash chromatography eluting with diethyl ether-hexanes [1) 50:50; 2) 100:0] to afford ethyl ester Ic-14 (10 mg, 43%) as well as 5 mg of lactone 14 (25%). Corrected yield=59%. White solid. Mp: 118-120° C. [α]_{D}²⁰=−5.4° (c1.0, in MeOH ). ¹H NMR (400 MHz, CD₃OD) δ 7.23 (m, 2H, 2×ArH), 7.15 (m, 3H, 3×ArH), 6.45 (s, 1H, H-3), 4.46 (d, J=8.1 Hz, 1H, H-7), 4.16 (m, 2H, OCH₂), 4.00 (ddd, J=12.1, 8.1 and 3.6 Hz, 1H, H-6), 3.03 (t, J=7.5 Hz, 2H, CH₂), 2.90 (m, 2H, CH₂), 2.35 (dd, J=13.3 and 12.1 Hz, 1H, H-5ₐₓ), 2.10 (dd, J=13.3 and 3.6 Hz, 1H, H-5_{eq}) and 1.21 (t, J=7.2 Hz, 3H, CH₃) ppm. ¹³C NMR (100 MHz, CD₃OD) δ 175.7 (C), 146.4 (C), 142.3 (C), 142.2 (C), 136.7 (C), 129.5 (2×CH), 129.4 (2×CH), 127.2 (CH), 123.3 (CH), 74.4 (C), 74.3 (CH), 71.6 (CH), 62.9 (OCH₂) 42.8 (CH₂), 38.9 (CH₂), 33.1 (CH₂) and 14.5 (CH₃) ppm. IR (KBr) 3456 (O—H), 3332 (O—H) and 1728 (C═O) cm⁻¹. MS (ESI) m/z (%): 385 (MNa⁺). HRMS calculated for C₁₉H₂₂O₅SNa (MNa⁺): 385.1080; found, 385.1070.

### Example 99

Methyl (4R,6R,7S)-4,6,7-trihydroxy-2-methyl-4,5,6,7-tetrahydrobenzo[b]thiophen-4-carboxylate (Ic-15). A solution of the lactone V-4 (example 35) (39 mg, 0.17 mmol) in dry methanol (1.7 mL) was treated with sodium methoxide (9 mg, 0.17 mmol). The resultant mixture was stirred at room temperature for 15 min. The reaction mixture was concentrated under reduced pressure. The obtained residue was dissolved in ethyl acetate and water. The organic layer was separated and the aqueous phase was extracted with ethyl acetate (×2). All the combined organic extracts were dried (anh. Na₂SO₄), filtered and concentrated under reduced pressure. The obtained residue was purified by flash chromatography eluting with ethyl acetate-hexanes [1) 75:25; 2) 100:0] to afford methyl ester Ic-15 (38 mg, 86%) as white solid. Mp: 87-92° C. [α]_{D}²⁰−8.2° (c1.0, in MeOH ). ¹H NMR (250 MHz, CD₃OD) δ 6.45 (s, 1H, ArH), 4.49 (d, J=8.1 Hz, 1H, H-7), 4.01 (ddd, J=11.8, 8.1 and 3.6 Hz, 1H, H-6), 3.74 (s, 3H, OMc), 2.40 (m, 4H, Me+H-5ₐₓ) and 2.12 (dd, J=13.7 and 3.6 Hz, 1H, H-5_{eq}) ppm. ¹³C NMR (75 MHz, CD₃OD) δ 176.2 (C), 142.2 (C), 141.6 (C), 136.8 (C), 124.0 (CH), 74.5 (C), 74.3 (CH), 71.6 (CH), 53.2 (OCH₃), 42.8 (CH₂) and 15.4 (CH₃) ppm IR (NaCl): 3420 (O—H) and 1736 (C═O) cm¹. MS (ESI) m/z (%) 281 (MNa⁺). HRMS calcd for C₁₁H₁₄O₅SNa (MNa): 281.0454; found, 281.0449.

### Example 100

(4R,6R,7S)-2-Ethyl-4,7-dihydroxy-4,5,6,7-tetrahydrobenzo[b]thiophen-4,6-carholactone (25). A suspension of vinyl lactonc V-6 (example 39) (40 mg, 0.17 mmol) and 20% palladium hydroxide-on-carbon (8 mg) in methanol (2.5 mL) was stirred under hydrogen atmosphere at room temperature for 12 h. The mixture was filtered over Celite and the residue was washed with methanol. The filtrate and washings were evaporated. The obtained residue was purified by flash chromatography eluting with diethyl ether-hexanes (70:30) to yield ethyl derivative 25 (37 mg, 90%) as white solid. Mp: 119-122° C. [α]_{D}²⁰−80.4° (c1.2, in MeOH). ¹H NMR (250 MHz, CD₃OD) δ 6.82 (s, 1H, H-3), 4.78 (m, 2H, H-7+H-6), 2.83 (q, J=7.5 Hz, 2H, CH₂CH₃), 2.55-2.43 (m, 2H, H-5) and 1.28 (t, J=7.5 Hz, 3H, CH_{;}) ppm. ¹³C NMR (75 MHz, CD₃OD) δ 178.4 (C), 150.2 (C), 142.0 (C), 133.5 (C), 119.9 (CH), 79.2 (CH), 75.0 (C), 66.7 (CH), 38.5 (CH₂), 24.5 (CH₂) and 16.5 (CH₃) ppm. IR (KBr): 3525 (O—H), 3458 (O—H), 1784 and 1761 (C═O) cm⁻¹.

### Example 101

Methyl (4R,6R,7S)-2-ethyl-4,6,7-trihydroxy-4,5,6,7-tetrahydro-benzo[b]thiophen-4-carboxylate (Ic-16). The experimental procedure used was the same as for methyl ester Ic-15 utilizing lactone 25 (24 mg, 0.10 mmol) in dry methanol (1 mL) and 5 mg of sodium methoxide (0.10 mmol). Purification by flash chromatography eluting with ethyl acetate-hexanes [1) 80:20; 100:0] afforded methyl ester Ic-16 (9 mg, 33%) as colourless oil. [α]_{D}²⁰−11.2° (c1.0, in MeOH ). ¹H NMR (400 MHz, CD₃OD) δ 6.47 (s, 1H, H-3), 4.49 (d, J=8.0 Hz, 1H, H-7), 4.01 (ddd, J=11.8, 8.1 and 3.6 Hz, 1H, H-6), 3.73 (s, 3H, OMe), 2.76 (q, J=7.5 Hz, 2H, OCH₂), 2.35 (m, 1H, H-5ₐₓ), 2.12 (dd, J=13.3 and 3.6 Hz, 1H, H-5,) and 1.25 (t, J=7.5 Hz, 3H, CH₃) ppm. ¹³C NMR (75 MHz, CD₃OD) δ 176.3 (C), 149.2 (C), 141.9 (C), 136.6 (C), 122.2 (CH), 74.6 (C), 74.3 (CH), 71.6 (CH), 53.2 (OCH₃), 42.8 (CH₂), 24.6 (CH₂) and 16.4 (CH₃) ppm. IR (film): 3373 (O—H) and 1736 (C═O) cm⁻¹. MS (ESI) m/z (%) 295 (MNa⁺). HRMS calcd for C₁₂H₁₆O₅SNa (MNa⁺): 295.0611; found, 295.0609.

### Example 102

Methyl (4R,6R,7S)-4,6,7-triacetoxy-2-methyl-4,5,6,7-tetrahydro-benzo[b]thiophen-4-carboxylate (26). A solution of the triol Ic-15 (16 mg, 0.06 mmol) in dry pyridine (1 mL) and acetic anhydride (1 mL) was stirred at room temperature for 36 h. The solvents were removed under reduced pressure and the obtained residue was purified by flash chromatography eluting with ethyl acetate-hexanes (50:50) to afford acetate 26 (20 mg, 90%) as yellow oil. [α]_{D}²⁰−100.4° (c2.2, in CHCl₃). ¹H NMR (250 MHz, CDCl₃) δ 6.78 (s, 1H, H-3), 6.04 (d, J=7.8 Hz, 1H, H-7), 5.42 (ddd, J=11.0, 7.8 and 4.1 Hz, 1H, H-6), 3.77 (s, 3H, OMe), 2.74 (dd, J=14.2 and 4.1 Hz, 1H, H-5_{eq}), 2.66 (dd, J=14.2 and 11.0 Hz, 1H, H-5ₐₓ), 2.41 (s, 3H, Me), 2.12 (s, 3H, Me), 2.10 (s, 3H, Me) and 2.05 (s, 3H, Me) ppm. ¹³C NMR (63 MHz, CDCl₃) δ 170.6 (C), 170.1 (C), 170.1 (C), 169.6 (C), 141.3 (C), 136.7 (C), 133.0 (C), 124.6 (CH), 76.9 (C), 70.4 (CH), 69.6 (CH), 53.0 (OCH₃), 34.6 (CH₂), 21.1 (CH₃), 21.0 (CH₃), 20.9 (CH₃) and 15.4 (CH₃) ppm. IR (NaCl): 1743 (C═O) cm⁻¹. MS (ESI) m/z (%) 407 (MNa^{′}). HRMS calcd for C₁₇H₂₀O₈SNa (MNa⁺): 407.0771; found, 407.0773.

### Example 103

Methyl (4R,6R,7S)-4,6,7-tributyroxy-2-methyl-4,5,6,7-tetrahydro-benzo[b]thiophen-4-carboxylate (27). A solution of triol Ic-15 (13 mg, 0.05 mmol), dry pyridine (24 μL, 0.30 mmol) and butyryl chloride (23 μL, 0.23 mmol) in dicloromethane (0.5 mL) at 0° C. was stirred during 12 h during which time the reaction mixtore reached room temperature. After this time more butyryl chloride (23 μL, 0.23 mmol) and dry pyridine (24 μL, 0.30 mmol) were added. After 8 h the reaction mixture was concentrated under reduced pressure and the obtained residue was purified by flash chromatography eluting with (30:70) ethyl acetate-hexanes to yield ester 27 (15 mg, 65%) as a colourless oil. [α]_{D}²⁰−71.5° (c1.1, in CHCl₃). ¹H NMR (250 MHz, CDCl₃) δ 6.77 (s, 1H, ArH), 6.07 (d, J=8.2 Hz, 1H, H-7), 5.43 (ddd, J=11.6, 8.2 and 3.7 Hz, 1H, H-6), 3.77 (s, 3H, OMe), 2.76 (dd, J=14.1 and 3.7 Hz, 1H, H-5_{eq}), 2.60 (dd, J=14.1 and 11.6 Hz, 1H, H-5ₐₓ), 2.51-2.19 (m, 9H, Mc+3×CH₂), 1.76-1.54 (m, 6H, 3×CH₂) and 1.00-0.84 (m, 9H, 3×CH₃) ppm. ¹³C NMR (63 MHz, CDCl₃) δ 173.2 (C), 172.8 (C), 172.7 (C), 169.7 (C), 141.2 (C), 137.0 (C), 132.9 (C), 124.6 (CH), 76.9 (C), 70.5 (CH), 69.4 (CH), 52.9 (OCH₃), 36.1 (CH₂), 36.1 (CH₂), 36.1 (CH₂), 34.9 (CH₂), 18.4 (CH₂), 18.3 (CH₂), 18.3 (CH₂), 15.5 (CH₃), 13.6 (CH₃), 13.6 (CH₃) and 13.5 (CH₃) ppm. IR (NaCl): 1741 (C═O) cm⁻¹. MS (ESI) m/z (%) 491 (MNa⁺). HRMS calcd for C₂₃H₃₂O₈SNa (MNO: 491.1710; found, 491.1713.

### Example 104

Methyl (4R,6R,7S)-4,6,7-tripentyroxy-2-methyl-4,5,6,7-tetrahydro-benzo[b]thiophen-4-carboxylate (28). The experimental procedure used was the same as for ester 27 utilizing triol Ic-15 in dicloromethane (0.6 mL) and dry pyridine (24 μL, 0.30 mmol) and pentanoyl chloride (27 μL, 0.23 mmol). Yield: 19 mg (73%). Colourless oil. [α]_{D}²⁰−49.8° (c1.8, in CHCl₃). ¹H NMR (250 MHz, CDCl₃) δ 6.77 (d, J=1.0 Hz, 1H, ArH), 6.06 (d, J=7.8 Hz, 1H, H-7), 5.42 (ddd, J=11.8, 7.8 and 3.8 Hz, 1H, H-6), 3.77 (s, 3H, OMe), 2.75 (dd, J=14.1 and 3.8 Hz, 1H, H-5_{eq}), 2.59 (dd, J=14.1 and 11.8 Hz, 1H, H-5ₐₓ), 2.50-2.20 (m, 9H, Me+3×CH₂), 1.72-1.50 (m, 6H, 3×CH₂), 1.46-1.16 (m, 6H, 3×CH₂) and 0.95-0.87 (m, 9H, 3×CH₃) ppm. ¹³C NMR (63 MHz, CDCl₃) δ 173.4 (C), 172.9 (C), 172.8 (C), 169.7 (C), 141.1 (C), 137.0 (C), 132.9 (C), 124.7 (CH), 76.8 (C), 70.5 (CH), 69.3 (CH), 52.9 (OCH₃), 34.9 (CH₂), 34.0 (CH₂), 34.0 (CH₂), 33.9 (CH₂), 26.9 (CH₂), 26.9 (CH₂), 26.8 (CH₂), 22.2 (CH₂), 22.2 (CH₂), 22.1 (CH₂), 15.4 (CH₃) and 13.7 (3×CH₃) ppm. IR (NaCl): 1743 (C═O) cm⁻¹. MS (ESI) m/z (%) 533 (MNa⁺). HRMS calcd for C₂₆H₃₈O₈SNa (MNa¹): 533.2180; found, 533.2181.

### Example 105

Methyl (4R,6R,7S)-2-[(E)-2-cyclopropy]vinyl-4,6,7-trihydroxy-4,5,6,7-tetrahydrobenzo[b]thiophen-4-carboxylate (Ic-17). The experimental procedure used was the same as for compound Ic-16 using compound V-12 (example 48) (40 mg, 0.14 mmol), sodium methoxide (8 mg) and methanol (1.4 mL). Colourless oil [α]_{D}²⁰+44.0” (c1.1, in CH₃OH). ¹H NMR (250 MHz, CD₃OD) δ 6.52 (s, 1H, ArH), 6.49 (d, J=15.5 Hz, 1H, CH═CHAr), 5.54 (dd, J=15.5 and 9.0 Hz, 1H, CH═CHAr), 4.49 (d, J=8.1 Hz, 1H, H-7), 4.02 (ddd, J=8.1, 3.7 and 12.0 Hz, 1H, H-6), 3.75 (s, 3H, OMe), 2.35 (dd, J=13.4 and 12.0 Hz, 1H, H-5ₐₓ), 2.13 (dd, J=13.4 and 3.7 Hz, 1H, H-5_{eq}), 1.49 (m, 1H, CH), 0.79 (m, 2H, CH₂) and 0.46 (m, 2H, CH₂) ppm. ¹³C NMR (63 MHz, CD₃OD) δ 176.1 (C), 144.9 (C), 141.7 (C), 137.2 (C), 136.2 (CH), 122.5 (CH), 122.0 (CH), 74.4 (C), 74.2 (CH), 71.5 (CH), 53.3 (OCH₃), 42.7 (CH₂), 15.2 (CH) and 7.7 (2×CH₂) ppm. IR (KBr): 3409 (O—H) and 1738 (C═O) cm⁻¹. MS (ESI) m/z (%) 333 (MNa⁺). HRMS calcd for C₁₅H₁₈O₅SNa (MNa⁺): 333.0767; found, 333.0770.

### Example 106

Methyl (4R,6R,7S)-2-[(E)-2-cyclopropyl]vinyl-4,6,7-triacetoxy-4,5,6,7-tetrahydrobenzo[b]thiophen-4-carboxylate (29). The experimental procedure used was the same as for compound 26 using triol Ic-17 (13 mg, 0.04 mmol), acetic anhydride (1 mL) and pyridine (1 mL). Colourless oil. [α]_{D}²⁰−23.3″ (c1.1, in CHCl₃). ¹H NMR (400 MHz, CDCl₃) δ 6.82 (s, 1H, H-3), 6.45 (d, J=15.6 Hz, 1H, CH═CHAr), 6.03 (d, J=8.0 Hz, 1H, H-7), 5.55 (dd, J=8.8 and 15.6 Hz, 1H, CH═CHAr), 5.41 (ddd, J=8.0, 3.6 and 11.2 Hz, 1H, H-6), 3.77 (s, 3H, OMe), 2.74 (dd, J=14.4 and 3.6 Hz, 1H, H-5_{eq}), 2.62 (dd, 1H, J=14.4 and 11.2 Hz, H-5ₐₓ), 2.13 (s, 3H, CH₃), 2.10 (s, 3H, CH₃), 2.05 (s, 3H, CH₃), 1.52-1.43 (m, 1H, CH), 0.80 (m, 2H, CH₂) and 0.47 (m, 2H, CH₂) ppm. ¹³C NMR (100 MHz, CDCl₃) δ 170.6 (C), 170.1 (C), 170.0 (C), 169.4 (C), 144.1 (C), 136.6 (CH), 135.8 (C), 133.4 (C), 122.8 (CH), 120.1 (CH), 76.8 (C), 70.4 (CH), 69.5 (CH), 53.3 (OCH₃), 34.6 (CH₂), 21.0 (CH₃), 20.9 (CH₃), 20.9 (CH₃), 14.3 (CH), 7.5 (CH₂) and 7.4 (CH₂) ppm. IR (KBr): 1743 (C═O) cm⁻¹. MS (ESI) m/z (%) 459 (MNa⁺). HRMS calcd for C₂₁H₂₄O₈SNa (MNa⁺): 459.1084; found, 459.1070.

### Example 107

Methyl (4R,6R,7S)-2-[(E)-prop-1-enyl]-4,6,7-trihydroxy-4,5,6,7-tetrahydrobenzo[b]thiophen-4-carboxylate (Ic-18). The experimental procedure used was the same as for compound Ic-16 using compound V-8 (example 42) (40 mg, 0.16 mmol), sodium methoxide (8 mg) and methanol (1.6 mL). Yield: 42 mg (93%). Colourless oil. [α]_{D}²⁰=+12.4° (c1.0, in MeOH). ¹H NMR (250 MHz, CD₃OD) δ 6.54 (s, 1H, H-3), 6.44 (dd, J=15.6 and 1.3 Hz, 1H, CH═CHAr), 6.02 (dq, J=15.6 and 6.6 Hz, 1H, CH═CHAr), 4.50 (d, J=8.1 Hz, 1H, H-7), 4.02 (ddd, J=8.1, 3.7 and 11.9 Hz, 1H, H-6), 3.75 (s, 3H, OMe), 2.35 (dd, J=13.3 and 11.9 Hz, 1H, H-5ₐₓ), 2.13 (dd, J=13.3 and 3.7 Hz, 1H, H-5_{eq}) and 1.82 (dd, J=6.6 and 1.3 Hz, 3H, CH₃) ppm. ¹³C NMR (63 MHz, CD₃OD) δ 176.1 (C), 144.8 (C), 142.2 (C), 137.1 (C), 126.8 (CH), 125.7 (CH), 123.0 (CH), 74.4 (C), 74.2 (CH), 71.5 (CH), 53.3 (OCH₃), 42.7 (CH₂) and 18.4 (CH₃) ppm. IR (KBr): 3388 (O—H) and 1736 (C═O) cm⁻¹. MS (ESI) m/z (%) 307 (MNa). HRMS calcd for C₁₃H₁₆O₅SNa (MNO: 307.0611; found, 307.0611.

### Example 108

Methyl (4R,6R,7S)-2-[(E)-prop-1-enyl]-4,6,7-triacetoxy-4,5,6,7-tetrahydrobenzo[b]thiophen-4-carboxylate (30). The experimental procedure used was the same as for compound 26 using compound Ic-18 (22 mg, 0.07 mmol), acetic anhydride (1 mL) and pyridine (1 mL). Yield: 23 mg (74%). White solid. Mp: 140-143° C. [α]_{D}²⁰−56.0° (c1.9, in CHCl₃). ¹H NMR (250 MHz, CDCl₃) δ 6.84 (s, 1H, H-3), 6.41 (dd, J=15.6 and 1.1 Hz, 1H, MeCH═CH), 6.11-5.96 (m, 2H, MeCH═CH+H-7), 5.42 (ddd, J=7.8, 4.0 and 11.1 Hz, 1H, H-6), 3.77 (s, 3H, OMe), 2.74 (dd, J=14.2 and 4.0 Hz, 1H, H-5_{eq}), 2.63 (dd, J=14.2 and 11.1 Hz, 1H, H-5ₐₓ), 2.14 (s, 3H, AcO), 2.11 (s, 3H, AcO), 2.06 (s, 3H, AcO) and 1.82 (dd, J=6.6 and 1.1 Hz, 3H, CH₃) ppm. ¹³C NMR (63 MHz, CD₃OD) δ 170.6 (C), 170.1 (C), 170.1 (C), 169.4 (C), 144.1 (C), 136.2 (C), 133.3 (C), 127.5 (CH), 123.8 (CH), 123.3 (CH), 76.8 (C), 70.4 (CH), 69.5 (CH), 53.0 (OCH₃), 34.6 (CH₂), 21.0 (CH₃), 21.0 (CH₃), 20.9 (CH₃) and 18.3 (CH₃) ppm. IR (KBr): 1745 (C═O) cm⁻¹. MS (ESI) m/z (%) 433 (MNa⁺). HRMS calcd for C₁₉H₂₂O₈SNa (MNa⁺): 433.0928; found, 433.0913.

### Example 109

Methyl (4R,6R,7S)-4,6,7-trihytiroxy-2-styryl-4,5,6,7-tetrahydrobenzo[b]thiophen-4-carboxylate (Ic-19). The experimental procedure used was the same as for compound Ic-16 using compound 11 (example 85) (40 mg, 0.13 mmol), sodium methoxide (7 mg) and methanol (1.3 mL). Yield: 39 mg (89%). Yellow oil. [α]_{D}²⁰+56.2° (c0.8, in CH₃OH). ¹H NMR (250 MHz, CDCl₃) δ 7.47 (d, J=7.3 Hz, 2H, 2×ArH), 7.32 (m, 2H, 2×ArH), 7.22 (m, 2H, ArH+PhCH═CH), 6.87 (d, J=16.2 Hz, 1H, CH═CHPh), 6.81 (s, 1H, H-3), 4.55 (d, J=8.1 Hz, 1H, H-7), 4.06 (ddd, J=11.9, 8.1 and 3.7 Hz, 1H, H-6), 3.77 (s, 3H, OMe), 2.39 (dd, J=13.4 and 11.9 Hz, 1H, H-5ₐₓ) and 2.17 (dd, J=13.4 and 3.7 Hz, 1H, H-5_{eq}) ppm. ¹³C NMR (63 MHz, CDCl₃) δ 175.0 (C), 143.6 (C), 142.6 (C), 137.2 (C), 136.6 (C), 128.8 (2×CH), 128.5 (CH), 127.8 (CH), 126.4 (2×CH), 124.2 (CH), 121.8 (CH), 73.4 (C), 73.3 (CH), 70.5 (CH), 52.3 (OCH₃) and 41.7 (CH₂) ppm. IR (film) 3390 (O—H) and 1736 (C═O) cm⁻¹. MS (ESI) m/z (%): 369 (MNa⁺). HRMS calculated for C₁₈H₁₈O₅SNa (MNa): 369.0767; found, 369.0767.

### Example 110

Methyl (4R,6R,7S)-4,7-dihydroxy-2-styryl-4,5,6,7-tetrahydro-benzo[b]thiophen-4-carboxylate (Ic-20). The experimental procedure used was the same as for compound 26 using compound Ic-19 (24 mg, 0.06 mmol), acetic anhydride (1 mL) and pyridine (1 mL). Yield: 27 mg (84%). Beige solid. Mp: 160-163° C. [α]_{D}²⁰=7.4° (c1.2, in CHCl₃). ¹H NMR (250 MHz, CDCl₃) δ 7.36 (d, J=7.3 Hz, 2H, 2×ArH), 7.30-7.08 (m, 3H, 3×ArH), 7.05 (d, J=16.1 Hz, 1H, PhCH═CH), 7.02 (s, 1H, H-3), 6.80 (d, J=16.1 Hz, 1H, CH═CHPh), 6.02 (d, J=7.8 Hz, 1H, H-7), 5.40 (ddd, J=11.1, 7.8 and 3.9 Hz, 1H, H-6), 3.73 (s, 3H, OMe), 2.70 (dd, J=14.2 and 3.9 Hz, 1H, H-5_{eq}), 2.57 (dd, J=14.2 and 11.1 Hz, 1H, H-5ₐₓ), 2.10 (s, 3H, AcO), 2.06 (s, 3H, AcO) and 2.01 (s, 3H, AcO) ppm. ¹³C NMR (63 MHz, CDCl₃) δ 170.6 (C), 170.1 (C), 170.0 (C), 169.4 (C), 143.8 (C), 137.4 (C), 136.3 (C), 129.6 (CH), 128.7 (2xCH), 128.0 (CH), 126.4 (2×CH), 125.5 (CH), 121.0 (CH), 76.7 (C), 70.4 (CH), 69.4 (CH), 53.0 (OCH₃), 34.8 (CH₂), 21.0 (CH₃), 21.0 (CH₃) and 20.9 (CH₃) ppm. IR (KBr) 1743 (C═O) cm⁻¹. MS (ESI) m/z (%): 495 (MNa⁻). HRMS calculated for C₂₄H₂₄O₈SNa (MNa⁻): 495.1084; found, 495.1067.

### Example 111

(1R,4S,5R)-1,4-Dihydroxy-3-(2-naphyl)methoxycyclohex-2-en-1,5-carbolactone (31) and (1R,4S,5R)-1,4-dihydroxy-3-(2-naphyl)methoxy-2-(2-naphyl)methylcyclohex-2-en-1,5-carbolactone (32). To a solution of KHMDS (4 mL, 2.0 mmol, 0.5 M in toluene) in dry DMF (2 mL), under argon and at −78° C., a solution of (1R,4S,5R)-1,4-di(tert-butyldimethylsilyloxy)-3-oxocyclohexan-1,5-carbolactone (400 mg, 1.00 mmol) in 10 mL of a mixture of DMF and toluene (1:1), both dry, was added. The resultant solution was stirred at this temperature for 30 min. A solution of 2-(bromomethyl)naphtalene (442 mg, 2.00 mmol) in 8 mL of a mixture DMF and toluene (3:2), both dry, was then added. After 1 h, water and brine were added. The aqueous phase was extracted with diethyl ether (3×2 mL). All the combined organic extracts were dried (anh. MgSO₄), filtered and concentrated. The obtained residue was purified by flash chromatography over silica gel eluting with diethyl ether/hexanes [1°) 5:95; 2°) 10:90] to afford 57 mg (10%) of (1R,4S,5R)-1,4-di(tert-butyldimethylsilyloxy)-3-(2-naphyl)methoxycyclohex-2-en-1,5-carbolactone and 75 mg (11%) of (1R,4S,5R)-1,4-di(tert-butyldimethylsilyloxy)-3-(2-naphyl)methoxy-2-(2-naphyl)methylcyclohex-2-en-1,5-carbolactone. Both compounds were deprotected using the same experimental procedure as for compound IV-3 (example 18) to afford diol 31 (20 mg, 61%) and diol 32 (32 mg, 64%), respectively.

Data for diol 31: Colourless oil. [α]_{D}²⁰=−112° (c1.0, in MeOH). ¹H NMR (250 MHz, CD₃OD) δ 7.79 (m, 4H, 4×ArH), 7.44 (m, 3H, 3×ArH), 5.01 (s, 1H, H-2), 4.91 (s, 2H, OCH₂Ar), 4.59 (m, 1H, H-5), 4.11 (d, 1H, J=3.5 Hz, H-4) and 2.27 (m, 2H, H-6) ppm. ¹³C NMR (63 MHz, CD₃OD) δ 179.3 (C), 155.8 (C), 135.3 (C), 134.8 (C), 134.6 (C), 129.2 (CH), 129.0 (CH), 128.7 (CH), 127.6 (CH), 127.3 (CH), 127.2 (CH), 126.6 (CH), 105.1 (CH), 77.0 (CH), 73.0 (C), 70.9 (CH₂), 67.8 (CH) and 38.4 (CH₂) ppm. IR (film) 3446 (O—H) and 1770 (C═O) cm⁻¹. MS (ESI) m/z (%) 335 (MNa⁺). HRMS calcd for C₁₈H₁₆O₅Na (MNa⁺): 335.0890; found, 335.0889.

Data for diol 32: White solid. Mp: 203-207° C. [α]_{D}²⁰=−190° (c1.4, in acetone). ¹H NMR (250 MHz, acetonc-d₅) δ 7.89-7.65 (m, 8H, 8×ArH), 7.53-7.37 (m, 6H, 6×ArH), 5.33 (d, 1H, J=11.8 Hz, CHHO), 5.15 (d, 1H, J=11.8 Hz, CHHO),4.69 (m, 2H, H-4+H-5), 3.92 (d, 1H, J=14.5 Hz, CHHAr), 3.74 (d, 1H, J=14.5 Hz, CHHAr), 2.53 (d, 1H, J=11.0 Hz, H-6_{eq}) and 2.41 (dd, 1H, J=11.0 and 5.7 Hz, H-6ₐₓ) ppm. ¹³C NMR (63 MHz, acetone-d₅) δ 177.8 (C), 149.8 (C), 140.1 (C), 140.1 (C), 137.1 (C), 135.2 (C), 134.9 (C), 134.6 (C), 133.6 (C), 129.5 (CH), 129.5 (CH), 129.4 (CH), 129.1 (CH), 129.0 (CH), 128.9 (CH), 128.7 (CH), 128.4 (CH), 127.7 (CH), 127.6 (CH), 127.5 (CH), 127.2 (CH), 127.0 (CH), 126.4 (CH), 76.7 (CH), 74.3 (C), 71.4 (CH₂), 66.9 (CH), 38.9 (CH₂) and 31.2 (CH₂) ppm. IR (KBr) 3460 (0-H), 3346 (O—H) and 1768 (C═O) cm⁻¹. MS (ESI) m/z (%) 475 (MNa⁺). HRMS calcd for C₂₉H₂₄O₅Na (MNa): 475.1516; found, 475.1511.

### Example 112

Sodium (1R,4S,5R)-1,4-trihydroxy-3-(2-naphyl)methoxycyclohex-2-en-1-carboxylate (Ib-10). The same experimental procedure was the same as for compound Ib-1 (example 19) using carbolactone 31 (8 mg, 0.02 mmol) in 0.2 mL of THF and 51 μL of NaOH (aq.). Yield=9 mg (99%). Beige solid. Mp: 54° C. (dec.). [α]_{D}²⁰=−12° (c1.1, in H₂O). ¹H NMR (300 MHz, 50% CD₃OD/D₂O) δ 7.93 (m, 4H, 4×ArH), 7.57 (m, 3H, 3×ArH), 5.04 (d, J=11.7 Hz, 1H, CHHO), 4.97 (d, J=11.7 Hz, 1H, CHHO), 4.95 (s, 1H, H-2), 4.09 (d, 1H, J=6.0 Hz, H-4), 4.01 (m, 1H, H-5), 2.16 (dd, J=13.8 and 8.1 Hz, 1H, H-6ₐₓ) and 2.06 (dd, 1H, J=13.8 and 3.9 Hz, H-6_{eq}) ppm. ¹³C NMR (75 MHz, 50% CD₃OD/D₂O) δ 182.1 (C), 157.0 (C), 135.4 (C), 134.3 (C), 134.1 (C), 129.2 (CH), 128.9 (CH), 128.6 (CH), 127.5 (CH), 127.4 (CH), 127.3 (CH), 126.8 (CH), 102.2 (CH), 74.8 (CH), 72.8 (C), 70.9 (CH), 70.6 (CH₂) and 38.7 (CH₂) ppm. IR (KBr) 3435 (O—H) and 1660 (C═O) cm⁻¹. MS (ESI) m/z (%) 375 (MNa⁺). HRMS calcd for C₁₈H₁₁O₆Na₂ (MNa⁺): 375.0815; found, 375.0817.

### Example 113

Sodium (1R,4S,5R)-1,4,5-trihydroxy-3-(naphth-2-yl)methoxy-2-(naphth-2-yl)methylcyclohex-2-en-1-carboxylate (Ib-11). The same experimental procedure was the same as for compound Ib-1 (example 19) using carbolactone 32 (29 mg, 0.06 mmol) in 0.6 mL of THF and 120 μL of NaOH (aq.). Yield=28 mg (95%). White solid. Mp: 184-187° C. [α]_{D}²⁰=4° (c1.1, in H₂O). ¹H NMR (250 MHz, 50% D₂O/CD₃CN) δ 7.76-7.57 (m, 6H, 6×ArH), 7.46-7.29 (m, 7H, 7×ArH), 7.15 (br d, J=8.2 Hz, 1H, ArH), 4.92 (d, J=11.2 Hz, 1H, CHHO), 4.71 (d, J=11.2 Hz, 1H, CHHO), 4.33 (d, J=3.0 Hz, 1H, H-4), 3.88 (m, 1H, H-5), 3.60 (d, J=15.7 Hz, 1H, CHHAr), 3.22 (d, J=15.7 Hz, 1H, CHHAr) and 2.10 (m, 2H, H-6) ppm. ¹³C NMR (63 MHz, 50% D₂O/CD₃CN) δ 180.5 (C), 151.9 (C), 140.1 (C), 136.0 (C), 134.2 (C), 133.7 (C), 133.5 (C), 132.5 (C), 128.7 (CH), 128.6 (CH), 128.5 (CH), 128.3 (CH), 128.2 (2xCH), 127.9 (CH), 127.3 (CH), 127.1 (CH), 126.9 (CH), 126.8 (CH), 126.8 (CH), 126.5 (CH), 125.7 (CH), 122.3 (C), 76.9 (C), 70.8 (CH), 70.4 (CH₂), 69.2 (CH), 36.8 (CH₂) and 32.7 (CH₂) ppm. IR (KBr) 3435 (O—H) and 1660 (C—O) cm⁻¹. MS (ESI) m/z (%) 493 (MH⁺). HRMS calculated for C₂₉H₂₆O₆Na (MH⁺): 493.1622; found, 493.1620.

### Example 114

(1R,4S,5R)-1,4-di(tert-butylclimethylsilyloxy)-2-(benzo[b]thiophen-5-yl)methyl-3-(thien-2-yl)methoxycyclohex-2-en-1,5-carbolactone (33). A solution of (1R, 4S,5R)-1,4-di(tert-butyldimethylsityloxy)-3-oxocyclohexan-1,5-carbolactone (350 mg, 0.87 mmol) in 17.5 mL of dry THF was treated with LHMDS (1.3 mL, 1.31 mmol) at room temperature. After 1 h, 2-iodomethylbenzothiophene (480 mg, 1.75 mmol) was added and the resultant mixture was stirred for 40 min. The reaction mixture was diluted with water and diethyl ether. The organic layer was separated and the aqueous phase was extracted with diethyl ether (3×25 mL). All the combined organic extracts were dried (anh. Na₂SO₄), filtered and concentrated. The obtained residue was purified by flash chromatography over silica gel eluting with diethyl ether/hexanes [1°) 0:100; 2°) 10:90] to afford 158 mg (33%) of the corresponding alkylated ketone. Then, to a stirred solution of KHMDS (1.2 mL, 0.58 mmol, 0.5 M in toluene) in dry DMF (1.4 mL), under argon and at −78° C., a solution of the previously obtained ketone (158 mg) in 2.8 mL of a mixture of DMF and toluene (1:1), both dry, was added. The resultant solution was stirred at this temperature for 30 min. A solution of 2-(bromomethyl)thiophene (102 mg, 0.58 mmol) in 2.4 mL of a mixture DMF and toluene (1.4:1), both dry, was then added. After 1 h, water and brine were added. The aqueous phase was extracted with diethyl ether (3×2 mL). All the combined organic extracts were dried (anh. MgSO₄), filtered and concentrated. The obtained residue was purified by flash chromatography over silica gel eluting with diethyl ether/hexanes [1°) 5:95; 2°) 10:90] to afford 54 mg (29%). Colourless oil. [α]_{D}²⁰−111° (c1.3, in CHCl₃). ¹H NMR (250 MHz, CDCl₃) δ 7.55 (d, 1H, J=8.2 Hz, ArH), 7.43 (s, 1H, ArH), 7.20 (d, 1H, J=5.5 Hz, ArH), 7.11-6.97 (m, 5H, 5×ArH), 4.73 (d, 1H, J=12.2 Hz, OCHH), 4.68 (d, 1H, J=12.2 Hz, OCHH), 4.39 (dd, 1H, J=5.5 and 3.5 Hz, H-5), 4.26 (d, 1H, J=3.5 Hz, H-4), 3.63 (d, 1H, J=15.0 Hz, CHHAr), 3.49 (d, 1H, J=15.0 Hz, CHHAr), 2.37 (d, 1H, J=11.0 Hz, H-6ₐₓ), 2.25 (dd, 1H, J=11.0 and 6.0 Hz, H-6_{eq}), 0.79 (s, 9H, C(CH₃)₃), 0.54 (s, 9H, C(CH₃)₃), 0.04 (s, 3H, CH₃), 0.02 (s, 3H, CH₃), −0.05 (s, 3H, CH₃) and −0.15 (s, 3H, CH₃) ppm. ¹³C NMR (63 MHz, CDCl₃) δ 175.5 (C), 148.3 (C), 139.7 (C), 138.8 (C), 136.0 (C), 130.3 (C), 127.8 (CH), 126.8 (CH), 126.6 (CH), 126.0 (CH), 125.4 (CH), 124.5 (C), 123.7 (CH), 123.1 (CH), 121.8 (CH), 77.5 (C), 74.7 (CH), 67.4 (CH₂), 67.3 (CH), 37.6 (CH₂), 30.1 (CH₂), 25.7 (C(CH₃)₃), 25.4 (C(CH₃)₃), 18.0 (C(CH₃)₃), −3.3 (CH₃), −3.5 (CH₃) and −4.5 (2×CH₃) ppm. IR (film) 1799 (C═O) cm⁻¹. MS (ESI) m/z (%) 643 (MH). HRMS calculated for C₃₃H₄₇O₅S₂Si₂ (MH⁺): 643.2398; found, 643.2393.

### Example 115

(2R,4S,5R)-1,4-dihydroxy-3-(thien-2-yl)methoxy-2-(benzo[b]thiophen-2-yl)methylcyclohex-2-en-1,5-earbolactone (34). The experimental procedure used was the same as for compound IV-3 (example 18) using 40 mg (0.06 mmol) of ether 33, 140 μL of TBAF and 0.9 mL of THF. Yield=22 mg (88%). Yellow solid. Mp: 140-143° C. [α]_{D}²⁰ =−148” (c1.2, in MeOH). ¹H NMR (250 MHz, CD₃OD) δ 7.67 (d, 2H, J=8.2 Hz, 2×ArH), 7.44 (d, 1H, J=5.5 Hz, ArH), 7.36 (dd, J=5.0 and 1.2 Hz, 1H, ArH), 7.26 (br d, 1H, J=8.2 Hz, ArH), 7.22 (d, 1H, J=5.5 Hz, ArH), 6.97 (m, 2H, 2×ArH), 5.21 (d, 1H, J=12.2 Hz, OCHH), 5.09 (d, 1H, J=12.2 Hz, OCHH), 4.62 (m, 1H, H-5), 4.55 (d, 1H, J=3.5 Hz, H-4), 3.74 (d, 1H, J=14.0 Hz, CHHAr), 3.57 (d, 1H, J=14.0 Hz, CHHAr), 2.38 (d, 1 H, J=11.0 Hz, H-6ₐₓ) and 2.31 (dd, 1 H, J=11.0 and 5.2 Hz, H-6_{eq}) ppm. ¹³C NMR (63 MHz, CD₃OD) δ 178.9 (C), 148.4 (C), 141.2 (C), 141.1 (C), 138.5 (C), 138.0 (C), 128.1 (CH), 127.7 (CH), 127.4 (CH), 127.1 (CH), 127.0 (C+CH), 124.8 (CH), 124.7 (CH), 122.6 (CH), 76.9 (CH), 73.9 (C), 66.2 (CH₂), 65.8 (CH), 38.5 (CH₂) and 30.6 (CH₂) ppm. IR (KBr) 3483 (O—H), 3429 (O—H) and 1749 (C═O) cm⁻¹. MS (ESI) m/z (%) 437 (MNa⁺). HRMS calculated for C₂₁H₁₈O₅S₂Na (MNa⁻): 437.0488; found, 437.0481.

### Example 116

Sodium (1R,4S,5R)-1,4,5-trihydroxy-3-(thien-2-yl)methoxy-2-(benzo[b]thiophen-2-yl)methylcyclohex-2-en-1-carboxylate (Ib-12). The same experimental procedure was the same as for compound Ib-1 (example 19) using carbolactone 34 (20 mg, 0.05 mmol) in 0.5 mL of THF and 97 μL of NaOH (aq.). Yield=22 mg (97%). White solid. Mp: 191-194° C. [α]_{D}²⁰=−55° (c1.2, in H₂O). ¹H NMR (250 MHz, 50% D₂O/CD₃CN) δ 8.27 (d, 1H, J=8.5 Hz, ArH), 8.21 (br s, 1H, ArH), 8.01 (d, J=5.5 Hz, 1H, ArH), 7.82 (m, 3H, 3×ArH), 7.44 (m, 2H, 2×ArH), 5.51 (d, 1H, J=11.5 Hz, OCHH), 5.35 (d, 1H, J=11.5 Hz, OCHH), 4.84 (m, 1H, H-4), 4.41 (m, 1H, H-5), 4.16 (d, 1H, J=15.2 Hz, CHHAr), 3.66 (d, 1H, J=15.2 Hz, CHHAr) and 2.61 (m, 2H, H-6) opm. ¹³C NMR (63 MHz, 50% D₂O/CD₃CN) δ 180.5 (C), 151.4 (C), 140.7 (C), 140.5 (C), 138.5 (C), 137.4 (C), 127.9 (CH), 127.6 (CH), 127.2 (CH), 127.2 (CH), 126.7 (CH), 124.8 (CH), 124.2 (C), 123.7 (C), 122.4 (CH), 76.9 (C), 70.7 (CH), 69.5 (CH), 65.3 (CH₂), 37.2 (CH₂) and 32.4 (CH₂) ppm. IR (KBr) 3408 (O—H) and 1605 (C═O) cm⁻¹.

### Example 117

Effect of compounds of formula I of the invention on the catalytic activity of the type II dehydroquinase from various sources. The inhibitory potency of compounds I of the invention against type II dehydroquinases from Mycobacterium tuberculoris, Streptomyces coelicolor and Helicobacter pylori were assayed as it is described in the following articles: 1) Sanchez-Sixto, C.; Prazeres, V. F. V.; Castedo, L.; Lamb, H.; Hawkins, A. R.; Gonzalez-Bello, C. J. Med. Chem. 2005, 48, 4871; 2) Prazeres, V. F. V.; Sanchez-Sixto, C.; Castedo, L.; Lamb, H.; Hawkins, A. R.; Riboldi-Tunnicliffe; Coggins, J. R.; Lapthorn, A. J.; González-Bello, C. Chem Med Chem 2007, 2, 194; and 3) S\dot{a}nchez-Sixto, C.; Prazeres, V. F. V.; Castedo, L.; S. W. Suh, Lamb, H.; Hawkins, A. R.; Canada, F. J.; Jiménez-Barbero, J.; González-Bello, C. Chem Med Chem 2008, 3, 756. The inhibition data (Kᵢ) are summarized in Table 1.

TABLE 1
Inhibition constants Kᵢ (μM) of compounds I against
type II dehydroquinases from Mycobacterium tuberculosis,
Helicobacter pylori and Streptomyces coelicolor.^{a}

| Compound | M. tuberculo- | H. pylori | S. coeli- |
|---|---|---|---|
| (2R)-Ia-1 | 0.76 | 9.4 | 10.2 |
| (2S)-Ia-1 | 1.85 | 21.5 | 6.7 |
| (2R)-Ia-2 | 1.70 | 20 | 76 |
| (2R)-Ia-3 | 0.10 | 1.40 | 1.55 |
| (2S)-Ia-3 | 0.09 | 1.18 | 1.05 |
| (2R)-Ia-4 | 0.089 | 0.25 | 0.022 |
| (2S)-Ia-4 | 0.09 | 0.97 | 0.15 |
| (2R)-Ia-5 | 0.025 | 0.17 | 0.034 |
| (2S)-Ia-5 | 0.10 | 1.40 | 0.34 |
| (2R)-Ia-6 | 0.047 | 2.6 | 0.242 |
| (2S)-Ia-6 | 0.074 | 0.97 | 0.0065 |
| (2R)-Ia-7 | 0.029 | 0.16 | 0.004 |
| (2S)-Ia-7 | 0.055 | 0.90 | 0.038 |
| Ib-1 | 0.0032 | 0.16 | 0.00048 |
| Ib-2 | 0.00425 | 0.14 | 0.0028 |
| Ib-3 | 0.040 | 0.097 | 0.0024 |
| Ib-4 | 0.19 | 0.07 | 0.013 |
| Ib-5 | 1.24 | 1.10 | 0.015 |
| Ib-6 | | 0.92 | 0.14 |
| Ib-8 | | 15 | 0.14 |
| Ib-7 | 0.031 | 0.166 | 0.0003 |
| Ib-9 | | 0.140 | 0.024 |
| Ib-.10 | | | <0.00026 |
| Ib-11 | | | 0.032 |
| Ib-12 | | 0.046 | 0.279 |
| Ib-13 | | 0.021 | 0.280 |
| Ic-1 | 3.0 | 34 | 9.7 |
| Ic-2 | 0.40 | 1.60 | 3.15 |
| Ic-3 | 0.052 | 1.25 | 0.81 |
| Ic-4 | 0.097 | 0.35 | 0.093 |
| Ic-5 | 0.86 | 38 | 1.88 |
| Ic-6 | 0.11 | 1.08 | 0.0003 |
| Ic-7 | 0.097 | >150 | 0.085 |
| Ic-8 | 0.090 | 0.60 | 0.00085 |
| Ic-9 | 0.185 | 3.4 | 0.80 |
| Ic-10 | | 20.5 | 4.65 |
| Ic-11 | | 16 | 0.15 |
| Ic-12 | | 1.7 | 0.046 |
| Ic-13 | | 0.12 | 0.022 |
| Ic-15 | | | 0.0054 |

^{a}Assay conditions: pH = 7.0, 25° C., 50 mM Tris•HCl (H. pylori and S. coelicolor) or 50 mM Tris•HOAc (M. tuberculosis).

As shown in Table 1, the present invention provides inhibitors with inhibition constants as low as 3.2 nanomolar (Ib-1) or 25 nanomolar ((2R)-Ia-5) against Mycobacterium tuberculosis, the bacteria which causes tuberculosis. This represents 1600 times more affinity with respect of the natural substrate (Kₘ=40 and 8000 times more effective than 2,3-dehydroquinic acid (Kᵢ=200 μM). In the case of H. pylori affinities are as good as 21 nM (Ib-13). This represents about 21.000 times more affinity with respect of the natural substrate (Kₘ=444 μM), and 16.190 times more effective than the 2,3-dehydroquinic acid (Kᵢ=340 μM). In the case of S. coelicolor affinities arc as good as 0.3 nanomolar (Ic-6), 0.48 nanomolar (Ib-1) or 0.85 nanomolar (Ic-8).

1. A compound of formula I, its diastereoisomers, its enantiomers or its pharmaceutically acceptable salts or solvates 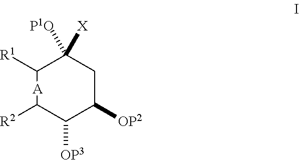
wherein,
A represents a single or double bond;
X is selected from the group consisting of —(C═O)OR^{a} and —(C═O)NR^{b}R^{c}, wherein each of R^{a}, R^{b} and R^{c} is independently selected from the group consisting of hydrogen, substituted or unsubstituted alkyl, substituted or unsubstituted alkenyl, substituted or unsubstituted alkynyl, substituted or unsubstituted aryl, substituted or unsubstituted heterocyclyl, substituted or unsubstituted arylalkyl and substituted or unsubstituted heterocyclylalkyl; or R^{b} and R^{c} together form a 5 or 6 membered heterocyclyc ring together with the nitrogen atom to which they are attached;
each P¹, P² and P³ is independently selected from the group consisting of hydrogen, substituted or unsubstituted alkyl, substituted or unsubstituted alkenyl, substituted or unsubstituted alkynyl, substituted or unsubstituted silyl, substituted or unsubstituted arylalkyl and —(C═O)R^{a}, wherein R^{a} is as defined above; and
wherein
if A is a double bond,
then R² is selected from the group consisting of —OR^{a}, —SR^{a} and —NR^{b}R^{c}, wherein R^{a}, R^{b} and R^{c} are as defined above; and R¹ is hydrogen or R^{1a}, wherein R^{1a} is selected from the group consisting of substituted or unsubstituted alkyl, substituted or unsubstituted alkenyl, substituted or unsubstituted alkynyl, substituted or unsubstituted aryl, substituted or unsubstituted heterocyclyl, substituted or unsubstituted arylalkyl and substituted or unsubstituted heterocyclylalkyl, with the proviso that:
when X is COOMe and P¹, R¹, P³ and P² are H, then R² is not methoxy,
when X is COOMe, P¹, P² and P³ are SiMe₃, and R¹ is H, then R² is not OH, and
when X is COOH and P¹, R¹, P³ and P² are H, then R² is not OH;
or
R¹ and R² together form a 5-membered ring; and
if A is a single bond,
then R² is ═O, ═S or ═NR^{b}, wherein R^{b} is as defined above; and R¹ is R^{1a}, wherein R^{1a} is as defined above.

2. A compound of formula Ic according to claim 1, its diastereoisomers, its enantiomers or its pharmaceutically acceptable salts or solvates 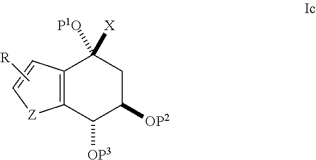
wherein,
X, P¹, P² and P³ are as defined in claim 1;
Z is selected from the group consisting of O, S, NR^{b} and ^{\textcircled{+}}NR^{b}R^{c}, wherein R^{b} and R^{c} are as defined in claim 1; and
R is selected from the group consisting of a hydrogen, halogen, substituted or unsubstituted alkyl, substituted or unsubstituted alkenyl, substituted or unsubstituted alkynyl, substituted or unsubstituted aryl, substituted or unsubstituted heterocyclyl, substituted or unsubstituted aryalkyl and substituted or unsubstituted heterocyclylalkyl; or
R^{b} and R together form a substituted or unsubstituted 4, 5, 6, 7 or 8 membered ring; or
a compound of formula Ia, its diastereoisomers, its enantiomers or its pharmaceutically acceptable salts or solvates 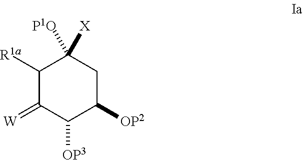
wherein X, P¹, P², P³ and R^{1a} are as defined in claim 1, and W is ═O, ═S or =NR^{b}, wherein R^{b} is as defined in claim 1.

3. A compound of formula Ib according to claim 1, its diastereoisomers, its enantiomers or its pharmaceutically acceptable salts or solvates 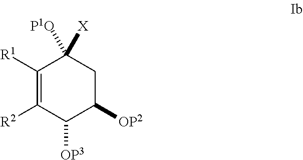
wherein,
X, P¹, P², P³ and R¹ are as defined in claim 1; and
R² is selected from the group consisting of —OR^{a}, —SR^{a} and —NR^{b}R^{c}, wherein R^{a},
R^{b} and R^{c} are as defined in claim 1, with the proviso that:
when X is COOMe and P¹, R², P³ and P² are H, then R² is not methoxy,
when X is COOMe and P¹, P² and P³ are Si Me₃, and R¹ is H, then R² is not OH, and
when X is COOH and P¹, R¹, P³ and P² are H, then R² is not OH.

4. A compound of formula I according to claim 1, selected from the group consisting of:
(2R)-2-allyl-3-dehydroquinic acid,
(2S)-2-allyl-3-dehydroquinic acid,
(2R)-2-propyl-3-dehydroquinic acid,
(2R)-2-benzyl-3-dehydroquinic acid,
(2S)-2-benzyl-3-dehydroquinic acid,
(2R)-2-(4-methyl)benzyl-3-dehydroquinic acid,
(2S)-2-(4-methyl)benzyl-3-dehydroquinic acid,
(2R)-2-(4-methoxy)benzyl-3-dehydroquinic acid,
(2S)-2-(4-methoxy)benzyl-3-dehydroquinic acid,
(2R)-2-perfluorobenzyl-3-dehydroquinic acid,
(2S)-2-perfluorobenzyl-3-dehydroquinic acid,
(2R)-2-(benzo[b]thiophen-5-yl)methyl-3-dehydroquinic acid,
(2S)-2-(benzo[b]thiophen-5-yl)methyl-3-dehydroquinic acid,
Sodium (1R,4S,5R)-3-(benzo[b]thiophen-2-yl)methoxy-1,4,5-trihydroxycyclohex-2-en-1-carboxylate,
Sodium (1R,4S,5R)-3-(benzo[b]thiophen-2-yl)methoxy-2-(benzo[b]thiophen-2-yl)methyl-1,4,5-trihydroxycyclohex-2-en-1-carboxylate,
Sodium (1R,4S,5R)-1,4,5-trihydroxy-3-(5-methylbenzo[b]thiophen-2-yl)methoxycyclohex-2-en-1-carboxylate,
Sodium (1R,4S,5R)-1,4,5-trihydroxy-3-(5-methylbenzo[b]thiophen-2-yl)methoxy-2-(5-methylbenzo[b]thiophen-2-yl)methylcyclohex-2-en-1-carboxylate,
Sodium (1R,4S,5R)-1,4,5-trihydroxy-3-(5-methylbenzo[b]thiophen-2-yl)methoxy-2-(5-methylbenzo[b]thiophen-2-yl)methylcyclohex-2-en-1-carboxylate,
Sodium (1R,4S,5R)-2-allyl-3-(benzo[b]thiophen-2-yl)methoxy-1,4,5-trihydroxycylohex-2-en-1-carboxylate,
(4R,6R,7S)-4,6,7-trihydroxy-4,5,6,7-tetrahydrobenzo[b]thiophen-4-carboxylic acid,
(4R,6R,7S)-4,6,7-trihydroxy-2-methyl-4,5,6,7-tetrahydrobenzo[b]thiophen-4-carboxylic acid,
(4R,6R,7S)-4,6,7-trihydroxy-2-vinyl-4,5,6,7-tetrahydrobenzo[b]thiophen-4-carboxylic acid,
(4R,6R,7S)-4,6,7-trihydroxy-2-[(E)-prop-1-enyl]-4,5,6,7-tetrahydro-benzo[b]thiophen-4-carboxylic acid,
(4R,6R,7S)-4,6,7-trihydroxy-2-(1-methyl)vinyl-4,5,6,7-tetrahydrobenzo[b]thiophen-4-carboxylic acid,
(4R,6R,7S)-2-[(E)-2-cyclopropyl]vinyl-4,6,7-trihydroxy-4,5,6,7-tetrahydrobenzo[b]thiophen-4-carboxylic acid,
(4R,6R,7S)-4,6,7-trihydroxy-2-phenyl-4,5,6,7-tetrahydrobenzo[b]thiophen-4-carboxylic acid,
(4R,6R,7S)-2-(2-cyclopropyl)ethyl-4,6,7-trihydroxy-4,5,6,7-tetrahydro-benzo[b]thiophen-4-carboxylic acid,
(4R,6R,7S)-4,6,7-trihydroxy-2-isopropyl-4,5,6,7-tetrahydrobenzo[b]thiophen-4-carboxylic acid,
(4R,6R,7S)-2-ethyl-4,6,7-trihydroxy-4,5,6,7-tetrahydrobenzo[b]thiophen-4-carboxylic acid,
Ethyl (1R,4S,5R)-2-allyl-3-(benzo[b]thiophen-2-yl)methoxy-1,4,5-trihydroxycyclohex-2-en-1-carboxylate,
Sodium (1R,4S,5R)-1,4,5-tridihydroxy-3-(thien-3-yl)methoxycyclohex-2-en-1-carboxylate,
Sodium (1R,4S,5R)-1,4,5-trihydroxy-3-[(benzo[b]thiophen-5-yl)methoxy]cyclohex-2-en-1-carboxylate,
Sodium (1R,4S,5R)-1,4-dihydroxy-3-(thien-23-yl)methoxy-2-(thien-23-yl)methylcyclohex-2-en-1-carboxylate,
Sodium (1R,4S,5R)-3-[(benzo[b]thiophen-5-yl)methoxy]-2-[benzo[b]thiophen-5-yl)methyl]-1,4-dihydroxycyclohex-2-en-1-carboxylate,
Methyl (1R,4S,5R)-3-(benzo[b]thiophen-5-yl)methoxy-2-(benzo[b]thiophen-5-yl)methyl-1,4,5-trihydroxycyclohex-2-enecarboxylate,
Methyl (1R,4S,5R)-1,4,5-tridihydroxy-3-(thien-3-il)methoxy-2-(thien-3-yl)methyl cyclohex-2-en-1-carboxylate,
Sodium (1R,4S,5R)-1,4,5-trihydroxy-3-(benzo[b]thiophen-5-yl)methoxy-2-(thien-2-yl)methylcyclohex-2-en-1-carboxylate,
Methyl (1R,4S,5R)-1,4,5-trihydroxy-3-(benzo[b]thiophen-5-yl)methoxycyclohex-2-en-1-carboxylate,
(4R,6R,7S)-4,6,7-trihydroxy-2-(1-phenylvinyl)-4,5,6,7-tetrahydro-benzo[b]thiophen-4-carboxylic acid,
(4R,6R,7S)-4,7-dihydroxy-2-styryl-4,5,6,7-tetrahydrobenzo[b]thiophen-4-carboxylic acid,
(4R,6R,7S)-4,6,7-trihydroxy-2-phenethyl-4,5,6,7-tetrahydroben-zo[b]thiophene-4-carboxylic acid,
(4R,6R,7S)-4,6,7-trihydroxy-2-propyl-4,5,6,7-tetrahydroben-zo[b]thiophene-4-carboxylic acid,
(4R,6R,7S)-2-Ethyl-4,6,7-trihydroxy-4,5,6,7-tetrahydrobenzo[b]tiophene-4-carboxylic acid,
(4R,6R,7S)-2-benzyl-4,6,7-trihydroxy-2-benzyl-4,5,6,7-tetrahydro-benzo[b]thiophen-4-carboxylic acid,
(4R,6R,7S)-4,6,7-trihydroxy-2-phenethyl-4,5,6,7-tetrahydro-benzo[b]thiophen-4-carboxylate,
Methyl (4R,6R,7S)-4,6,7-trihydroxy-2-methyl-4,5,6,7-tetrahydrobenzo[b]thiophen-4-carboxylate,
Methyl (4R,6R,7S)-2-ethyl-4,6,7-trihydroxy-4,5,6,7-tetrahydro-benzo[b]thiophen-4-carboxylate,
Methyl (4R,6R,7S)-2-[(E)-2-cyclopropyl]vinyl-4,6,7-trihydroxy-4,5,6,7-tetrahydrobenzo[b]thiophen-4-carboxylate,
Methyl (4R,6R,7S)-2-[(E)-prop-1-enyl[-4,6,7-trihydroxy-4,5,6,7-tetrahydrobenzo[b[thiophen-4-carboxylate,
Methyl (4R,6R,7S)-4,6,7-trihydroxy-2-styryl-4,5,6,7-tetrahydrobenzo[b[thiophen-4-carboxylate,
Methyl (4R,6R,7S)-4,7-dihydroxy-2-styryl-4,5,6,7-tetrahydro-benzo[b[thiophen-4-carboxylate,
Sodium (1R,4S,5R)-1,4-trihydroxy-3-(2-naphyl)methoxycyclohex-2-en-1-carboxylate,
Sodium (1R,4S,5R)-1,4,5-trihydroxy-3-(naphth-2-yl)methoxy-2-(naphth-2-yl)methylcyclohex-2-en-1-carboxylate,
Sodium (1R,4S,5R)-1,4,5-trihydroxy-3-(thien-2-yl)methoxy-2-(benzo[b]thiophen-2-yl)methylcyclohex-2-en-1-carboxylate,
or its enantiomers or its pharmaceutically acceptable salts or solvates.

5. Process for the preparation of compounds of formula Ia as defined in claim 2, comprising the ring opening of a lactone of formula III in acidic medium, 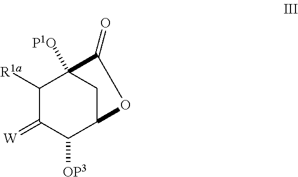
wherein, W, P¹, P³ and R^{1a} are as defined in claim 2.

6. Process for the preparation of a compound of formula Ib as defined in claim 3, comprising the ring opening of a lactone of formula IV in acidic or basic medium 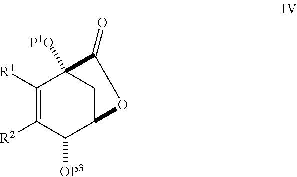
wherein, P¹, P³, R¹ and R² are as defined in claim 3.

7. Process according to claim 6, wherein the compound of formula IV is prepared by a process comprising an O-, S- or N-alkylation of a compound of formula III or of a compound of formula II, or a dialkylation of a compound of formula II, wherein P¹, P³, R¹ and R² are as defined in claim 6, and W and R^{1a} is as defined in claim 5

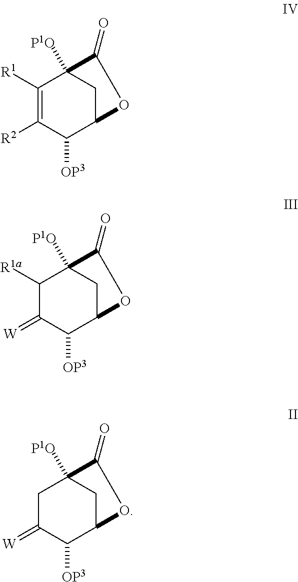

8. Process for the preparation of a compound of formula Ic, as defined in claim 2, comprising the ring opening of a lactone of formula V in acidic or basic medium, 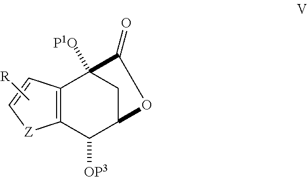
wherein, P¹, P³, R and Z are as defined in claim 2.

9. A compound of formula III, its diastereoisomers, its enantiomers or its pharmaceutically acceptable salts or solvates 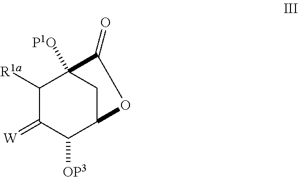
wherein W, P¹, P³ and R^{1a} are as defined in claim 5.

10. A compound of formula IV, its diastereoisomers, its enantiomers or its pharmaceutically acceptable salts or solvates 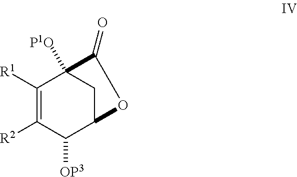
wherein P¹, P³, R¹ and R² are as defined in claim 6.

11. A compound of formula V, its diastereoisomers, its enantiomers or its pharmaceutically acceptable salts or solvates 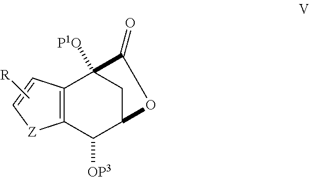
wherein P¹, P³, R and Z are as defined in claim 8.

12. A pharmaceutical composition comprising a compound of formula I as defined in claim 1 and a pharmaceutically acceptable carrier.

13. A compound of formula I as defined in claim 1, for use as a medicament.

14. A compound of formula I as defined in claim 1, for use as an antibiotic and/or antimicrobial.

15. A compound according to claim 14, for use in the treatment or prophylaxis of a disease selected from the group consisting of tuberculosis, stomach cancer, gastritis, stomach ulcers, and duodenal ulcers heartburn.

16. A compound of formula I, its diastereoisomers, its enantiomers or its pharmaceutically acceptable salts or solvates 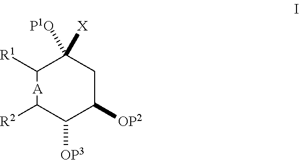
wherein,
A represents a single or double bond;
X is selected from the group consisting of —(C═O)OR^{a} and —(C═O)NR^{b}R^{c}, wherein each of R^{a}, R^{b} and R^{c} is independently selected from the group consisting of hydrogen, substituted or unsubstituted alkyl, substituted or unsubstituted alkenyl, substituted or unsubstituted alkynyl, substituted or unsubstituted aryl, substituted or unsubstituted heterocyclyl, substituted or unsubstituted arylalkyl and substituted or unsubstituted heterocyclylalkyl; or R^{b} and R^{c} together form a 5 or 6 membered heterocyclyc ring together with the nitrogen atom to which they are attached;
each P¹, P² and P³ is independently selected from the group consisting of hydrogen, substituted or unsubstituted alkyl, substituted or unsubstituted alkenyl, substituted or unsubstituted alkynyl, substituted or unsubstituted silyl, substituted or unsubstituted arylalkyl and —(C═O)R^{a}, wherein R^{a} is as defined above; and wherein
if A is a double bond,
then R² is selected from the group consisting of —OR^{a′}, —SR^{a} and —NR^{b}R^{c}, wherein R^{b} and R^{c} are as defined above; R^{a′} is selected from the group consisting of substituted or unsubstituted alkenyl, substituted or unsubstituted alkynyl, substituted or unsubstituted aryl, substituted or unsubstituted heterocyclyl, substituted or unsubstituted arylalkyl and substituted or unsubstituted heterocyclylalkyl; and R¹ is hydrogen or R^{1a}, wherein R^{1a} is selected from the group consisting of substituted or unsubstituted alkyl, substituted or unsubstituted alkenyl, substituted or unsubstituted alkynyl, substituted or unsubstituted aryl, substituted or unsubstituted heterocyclyl, substituted or unsubstituted arylalkyl and substituted or unsubstituted heterocyclylalkyl, or
R¹ and R² together form a 5-membered ring; and
if A is a single bond,
then R² is ═O, ═S or ═NR^{b}, wherein R^{b} is as defined above; and R¹ is R^{1a}, wherein R^{l}a is a defined above.

17. A compound of formula Ib according to claim 16, its diastereoisomers, its enantiomers or its pharmaceutically acceptable salts or solvates 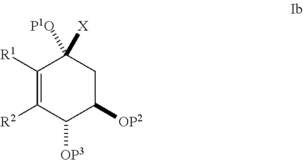
wherein:
X, P¹, P², P³ and R¹ are as defined in claim 16; and
R² is selected from the group consisting of —OR^{a′}, —SR^{a} and —NR^{b}R^{c}, wherein R^{a}, R^{b} and R^{c} are as defined in claim 16.
